(19) United States
(12) Patent Application Publication
(10) Pub. No.: US 2025/0341624 A1
(43) Pub. Date: Nov. 6, 2025

(54) MILLIMETER RADAR FOR INTERROGATION, CLASSIFICATION AND LOCALIZATION OF TARGET OBJECTS HAVING A NON-LINEAR FREQUENCY DEPENDENT FREQUENCY RESPONSE, ENHANCED BY WIDEBAND CHAOS GENERATING MATERIAL (WCGM)

(71) Applicant: Xfinder Sweden AB, Karlskrona (SE)

(72) Inventors: Per Ola CARLSSON, Lyckeby (SE); Kent JAKOBSSON, Sturkö (SE); Siamak KHATIBI, Rödeby (SE)

(73) Assignee: Xfinder Sweden AB, Karlskrona (SE)

(21) Appl. No.: 19/124,066

(22) PCT Filed: Oct. 23, 2023
(86) PCT No.: PCT/SE2023/051045

§ 371 Date: Apr. 24, 2025

(30) Foreign Application Priority Data
Oct. 25, 2022 (SE) 2251238-8

(51) Int. Cl. G01S 13/02; G01S 7/02; G01S 7/288; G01S 13/34

(52) U.S. Cl. CPC G01S 13/0209; G01S 7/023; G01S 7/2883; G01S 13/34

(57) ABSTRACT

A system for millimeter RADAR object recognition and classification using sub-band frequency interference and resonance effects from primary targets signals and reflected signals from secondary target preferably in the form of Wideband Chaos Generating Material (WCGM) objects, preferably detecting frequency dependent absorbing material, frequency dependent resonance effects from the second and primary target objects, frequency signal resonance effects caused by water molecule dipole effects in different sugar solutions, impedance of material, shape of metamaterial, and interference effects due to combination of signal sources resulting in a wider range of transmitter and scanning frequency band for RADAR based interrogation of target objects. The RADAR system makes opportunistic use of traditionally seen problematic interference signals, as extra signal sources providing extended range and frequency bandwidth for frequency-based interrogation of target object signatures in a frequency-intensity plane, a frequency-polarization plane, and a frequency-phase shift plane for doppler effects.

### TECHNICAL FIELD

RADAR signature interrogation and recognition system, and methods for recognition, classification, volume determination, remote substance analysis of target objects, localization using millimeter wavelengths; especially for target objects that returns a characteristic non-linear frequency response model when interrogated with a characteristic transmitter interrogation signal; using meta-materials and reflected distorted signals to enhance the RADAR's bandwidth and ability to recognize and classify, target objects, substances, volumes, and characteristics of target objects.

### BACKGROUND

RADAR techniques and methods are a highly developed technical discipline due to many earlier applications in surveillance and measuring system for warfare, aircraft industry, meteorology, and science. RADARs are involved in everyday use with applications for millimeter RADAR wavelength applications in automotive industry, as automatic door sensors, airport screening systems, sensors for handheld computers, and sensors in health care including sensors for monitoring of heartbeats. Millimeter and ultrawideband RADARs are introduced in autonomous robots, for Simultaneous Localization and Mapping (SLAM) navigation systems, and for tracking of goods and merchandises in grocery store's checkout systems. The list of millimeter RADAR applications is extensive.

Due to electromagnetic wave propagation physics, a RADAR transmitting its RADAR signal, as pulse or waveform, towards a target object to be detected, would in isolation receive a chirp from the target object having a RADAR Cross Section (RCS) that would be returned to the RADAR receiver, after a time-to-fly (TOF) corresponding to the distance from sender to target object and back to its receiver. The RCS return signal is influenced by factors such as: conductivity of the target, size, and geometries of the silhouette. A larger RCS area results in a stronger chirp signal returned to the RADAR receiver. This is the foundation for RADAR technologies in general, and for most millimeter wavelength RADAR systems, and their applications. RADARS uses signal strength and direction towards the target object to identify the relative location of a target object. RADAR signal beam forming, suing a sweeping antenna design with one, or multiple RADAR lobes, has been in use for RADAR scanning, and to follow target object's aspect view, RADAR cross section (RCS), or silhouette, as presented for the RADAR antenna. In its most simple form, a RADAR may be based on a traditional beam forming parabolic antenna with RADAR transmitter and receiving horn, or even as first described in Hülsmeyer's patent DE165546C, which described a funnel shaped transmitter antenna. The most basic antenna design is an omnidirectional antenna with no beamforming and no ability to recognize direction to target object, while distance recognition to target object, may be determined based on a TOF calculation of a first reflection with high signal strength.

RADAR antenna systems come in all sorts of configurations from Hülsmeyer's most basic single input single output (SISO) to multiple input multiple output (MIMO) antenna arrangements having sophisticated abilities to control transmitted RADAR lobe, and receiver's RADAR lobes, as well as to use signal processing to extract target chirp signals from a certain location in relation to the antenna arrangement. Also, multiple cooperating antennas configurations are possible for collaborative RADAR systems. A Synthetic-Aperture RADAR:s (SAR), can detect and generate two-dimensional images, or three-dimensional reconstructions of objects, such as landscapes.

RADAR waves may penetrate the human body, which makes it possible to use RADAR technology for non-invasive medical body measurements. Medical safety can be ensured by carefully selecting RADAR wavelengths, power level, and energy levels for the intended purpose. Different wave lengths with frequencies have the following properties:

| Frequency | | Wavelength | | Typical penetration depth into the human body |
|---|---|---|---|---|
| 3-7 | GHz | 4-10 | cm | Penetrates thru the whole body. Depending on the exposed time |
| | | | | where longer time results in higher energy and more heating and |
| | | | | have been found unsafe. |
| 20-24 | GHz | about 1 cm | | Penetrates deeper than the skin level. Long term exposure |
| | | | | depending on energy level may be unsafe, while not confirmed. |
| 68-78 | GHz | about 5 mm | | Penetrates only into the skin tissue and reflects, which still may be |
| | | | | useful for non-invasive measurements of the human body. This |
| | | | | frequency range is considered medical safe. |
| 79-120 | GHz | 4-2.5 | mm | Higher frequencies from 79 to 120 GHz reflects in the skin tissue |
| | | | | and can be used for measuring variations in movement of the skin |
| | | | | for heartbeat detection and for measuring breathing. |

Hence many millimeter RADAR applications are considered medical safe for the human body. A millimeter RADAR may have a different antenna, and lobe forming capabilities ranging from merely distance measuring time-of-flight TOF RADARs having a SISO antenna configuration, to SAR and MIMO antennas with multiple lobe support and advanced lobe shape and direction control both for the transmitter side and receiver side. Also, variations in between such as single input multiple output SIMO and multiple input single output MISO configurations exists. Cooperating RADAR systems are known for aircraft and automotive 360-degree RADAR systems where multiple target object detection signals are integrated during data fusion into a common operational picture.

As the RADAR to target object environment is rarely isolated from other objects reflecting signals in the environment, producing not only complex signal reflections, but also creating combinations of multiple signals viewed as signal interference. Also, a target object may provide multiple time-of-flight reflection planes for the same signal, thus resulting in multiple chirp signals to be analyzed. A time-of-flight pulse RADAR, with direction information from each RADAR lobe direction, can extract and separate several signal chirps during a target object interrogation cycle. For a millimeter RADAR using RADAR signal interrogation techniques such as Frequency Modulation Continuous Wave (FMCW), hundreds cycles of a Chirp signal containing a frequency sweep pattern such as: rising, falling, or triangular rising to peak and falling; would transmit several RADAR waves while constantly sampling received signals, and performing signal analysis of mixed transmitted and received signals in the RADAR signal processing system.

RADAR systems can be software defined radio (SDR) based, implemented in hardware, using a Field Programmable Gate Array (FPGA) which in turn may include digital signal processors (DSP), electronics, computers, just to mention a few RADAR control transceiver control systems. Usually in a millimeter RADAR, sampled mixed receiver and transmitter signals are processed by an optional bandpass filter before, and/or after entering a Fast-Fourier Transform (FFT) transformation that translate the mixed sample signal for each RADAR lobe from a time domain into the frequency domain, a frequency plane expressing signal magnitude, strengths, phase shift, doppler effects, and possibly signal polarization, as a function of frequency.

RADAR signatures and reflection characteristics from target objects does not only comprises mentioned RCS characteristics but may also in advanced RADAR systems comprise distance, angle of arrival, signal strength, and a frequency; also, doppler shift, which is frequency and phase shifts of received signals indicating a direction of travel way from or towards the antenna system. Other RADAR signal information such as a frequency fingerprint indicating a target object's ability to reflect specific frequencies, and the polarization of a received signal in relation to transmitted signal, are also signal characteristics.

A frequent problem for millimeter RADAR applications are noise and interference signals. Noise in high power RADAR systems may be a result due to intrinsic noise generated by the RADAR transceiver, receiver, amplification, and antenna electronics, but more often from the physical environment around RADAR and target object. Interference signals generated from disturbing target objects and the environment are usually reduced together using a bandpass filter, with the intention to improve a Signal to Noise Ratio (SNR). Hence, signal interferences are traditionally a problem for most RADAR systems.

The patent application WO2018206934A1, REAL-TIME LOCATION SENSING SYSTEM, describes a millimeter RADAR having interesting properties for relative localization of man-made RADAR tags, and target objects designed to reflect an FMCW RADAR signal interrogation signal. The type of interrogation signal used, a Chirp, is named after how it would sound if replicated as a sound. When the RADAR is interrogating a tag having a designed frequency dependent absorption filter array, absorbing certain frequencies, only a few discrete frequencies will be reflected with strength to the RADAR receiver. An alternative design is the use of a repeated resonance circuit in form of a RADAR tag, designed using metamaterial technologies. Each metamaterial RADAR tag can be designed to return signals at certain wavelengths to the RADAR receiver, while discriminating other frequencies. Using a tag designed to absorb a specified set of frequencies, the return signal will carry a fingerprint with discrete absorbed at a specified set of frequencies. Then the receiver would sample the receiver signal and transform the signal into the frequency domain via FFT, for further extraction of a digital fingerprint defined by the distinct specified set of frequencies.

To measure the identity of the RADAR tag, the system measures the amplitude of certain frequencies, about 1 to 7 frequencies, to determine if the identity should read as a binary 0 or binary 1, thus contributing to a readable identity digit of range 1-127. The decoding can be made using a set of bandpass filters tuned to the frequencies 1 to 7. This can be seen as a simple passive Radio Frequency IDentification (RFID) tag, transmitting multiple code values.

This system relies on designed RADAR tags for object identification and handles noise and interferences by sampling multiple FMCW signals and then averaging the signals, before performing a bandpass filtering to extract each distinct frequency component. As distance to RADAR tags varies, so varies the amplitude and intensity of a frequency reflected from its target object, being a designed RADAR tag. Mentioned patent application teaches that its RADAR tags may be designed using metamaterials in the form of a matrix surface of metallic resonating C:s with a smaller c:s inside the first C, thus forming an impedance circuit with abilities to resonate with certain frequencies. Hence, tags are decoded into binary numbers, a rigid decoding model accepting only those tags designed for the system in mind. Other target objects may be recognized using ordinary time of flight RADAR chirps using an FMCW RADAR. The system may also scan for FFT responses at a certain frequency by transmitting a Chirp at a certain frequency window, and reading out a specific frequency intensity or amplitude, which decodes as a binary 0 or 1; before moving to next frequency window and repeating the process.

RADAR systems are normally designed to overcome unwanted noise signals, disturbing the signal focus on target objects of interest. Unwanted signals may originate from internal and external sources, both passive and active. The ability of the radar system to overcome unwanted noise signals defines its signal-to-noise ratio (SNR). Temporary SNR problems are reduced in low power millimeter RADAR systems of FMCW type by oversampling over a signal capturing window. SNR is defined as the ratio of the signal power to the noise power from the desired signal; it compares the level of a desired target signal to the level of background noise not limited to atmospheric noise, and noise generated within by receiver amplifier. With a higher SNR, the better the system is it at discriminating actual targets from noise signals and signal interferences.

Another patent application WO2016205217A1 HIGH-PRECISION TIME OF FLIGHT MEASUREMENT SYSTEM, deals with challenges of multiple signal paths: from sender to target object, and returned to the receiver antenna, by modulating transmitter signals and providing a reference transmitter signal to resolve problems with multi-path signals. The same system is based on time-of-flight calculations and uses a band-pass filter to improve SNR.

For millimeter RADARs, the signal processing and analysis steps usually follows when a transmitter Tx has transmitted as a RADAR signal, a Chirp, directed towards a target object, and a RADAR reflection with possible chirps has been received from the target object, by a Rx antenna.

First the transmitter Tx signal is sent to a signal amplitude reducing filter, and then mixed with the receiver Rx antenna signal into a MIX signal. This MIX signal is then bandpass filtered to reduce noise and eliminate non-relevant signals disturbances before the filtered MIX signal is further analyzed.

The MIX signal is then analyzed to determine time-of-flight (TOF) to a target object in the time domain. A Fast Fourier Transform (FFT) transformation, transforms the information into a frequency domain to further allow matching of frequencies towards distances, thus being able to calculate distance to reflection of identical Tx and Rx signals in one or multiple transmitted Chirps.

This allows for analysis of distance mapping and identification of objects reflecting signals at certain wavelengths and frequencies. The method may also support further analysis of phase shifts, and doppler effects to identify moving targets having a radial velocity in relation to the RADAR antenna arrangement.

Two major challenges for previously mentioned millimeter RADAR systems are to securely identify a target object independently of the target object's distance to the radar and the target object's geometrical profile. A third challenge is to locate and identify the target object without reduced precision due to signal disturbances from signal interfering objects, and Wideband Chaos Generating Material (WCGM) contributing with interfering signals, and signal scatters within the RADAR frequency band in use.

It is usually a desired to improve and increase a RADAR system's SNR, to let it improve its ability to discriminate noise signals form actual target response signatures.

Some methods and problems are generic for RADAR technologies independent of radio wavelength, but the millimeter wavelength technology exposes new challenges and effects that are not present in for example shorter than 3 cm wavelength frequency band.

Frequencies between 1 GHz and 124 GHz used are commonly referred to as the “mm Wave wide-band”. Wavelength for mm Wave wide band frequency of 124 GHz is approximate 2.3 mm, meanwhile the “lower part of the mm Wave” band 4-7 GHz is approximate 3 cm. Wavelength of 7 GHz is 42.8 mm, meanwhile 4 GHz is approximate 75 mm.

RADAR signals will reflect on target objects having an at least diagonal RADAR Cross Section (RCS) of approximate: λ/2, where lambda (λ) is the wavelength of the RADAR signal. Hence a 124 GHz mm Wave wide band signal could reflect by a surface having an RCS cross diagonal length of 1.15 mm, meanwhile a 7 GHz would require a 21.4 mm. This means the distinguishable surface is depending on the signal frequency, where higher frequency makes it possible to distinguish smaller objects. Also, the RCS is important in this case (how the 3D object facing the RADAR. Other aspects that have an impact on reflected energy from a target object such as: material of target, size of target relative the wavelength, size of target, angle of target object, incident angel of target object and reflected angle, and polarization of RADAR signal transmitted and received. Also, the combined area of multiple target object may also contribute to a stronger reflected signal.

Lower frequency mm Wave of 4-7 GHz may penetrate thru the human a thin skin layer, and thru the belly of a human being. mm Wave wide band RADAR of frequencies 50 to 67 GHz has previous been used in sensors for measuring blood properties in laboratory environments. High frequency millimeter mm Wave signals beyond 124 GHz do not penetrate deeply into the skin layers. Meanwhile frequencies in the range of 3-77 GHz can be used for non-invasive body measurements measuring properties inside the human body. According to American safety guidelines, frequencies under 24 GHz has shown no safety problems such as heating in cells, with reasonable power levels. Upper frequencies such as from 55 GHz to 68 GHz may be in use for mobile applications. RADAR applications for automotive application, often use the frequency band from 51 GHz to 77 GHz.

Target objects for mm Wave RADAR:s comes in in many forms, and they become detectable when they appear in the field of view of the RADAR aperture and RADAR antenna array coverage. Metallic and other conductive object usually offers good reflectors, and for mm RADARS other non-metallic objects may also act as good target objects giving reasonable reflection. For tank radars, to measure fluids, one has traditionally measured the reflection, or time of flight. For fluid measurements of sugar-water solutions, mm RADAR applications are measuring the signal attenuation at a known distance at a certain frequency, as the signal attenuation effect in a sugar-water, as well as in a glucose-water solution in related to the sugar water concentration at a certain mm RADAR frequency. The sugar water concentration is denoted a Bx° Brix value. This type of sugar content measurements has been deployed in food industry when measuring a sugar content in a pipe flow. Industrial measurement sensors for sugar concentration in pipes and tanks uses the distance in combination with actual reflected signal strength at a certain frequency to determine Brix sensed values.

Target objects may also constitute RADAR reflectors, and arrangements of RADAR reflectors in geometrical structure to signal a certain recognizable object, or as reflector having a certain reflective profile shape being recognizable. It is however a challenge to recognize and classify irregular objects based on RCS when the target object rotates and constantly changes aspect angle towards the RADAR.

RADAR reflector beacons have been designed to return a signal with a predefined signal shift and time of flight delay for pattern matching for aircraft radars as a landing beacon support system but then for 3 to 15 cm wavelength RADAR applications.

Applications of mm RADAR techniques for a Real-time Location Sensing System (RTLS), is described in the patent application WO2018/206934. The RTLS described may make use of mm RADAR tags, a kind of small mm RADAR reflectors for tracking of assets, as well as active tags announcing their presence thru another radio wavelength and protocol such as over UHF presented as passive or active RFID tags. Alternatives mentioned are UHF RFID, or Bluetooth Low Energy (BLE) beacons, or Wi-Fi position systems (WiPS/WFPS). These solutions require more radio equipment than a millimeter RADAR, and active tags, which drives cost, and requires batteries.

Beyond active tags, the RTLS patent application mentions the use of passive tags, in the form of specifically designed metamaterial demonstrating certain electromagnetic properties.

Key to the technology of metamaterials, is the ability to design and engineer the electromagnetic response to a wave over the desired mm RADAR band. A metamaterial is a material in which its overall response may be designed to differ from that of its constituent materials; this ability is key because a tag may be fabricated out of copper and plastic, which have no special properties within the mm RADAR band beyond being conductors and insulators, respectively, however we may wish more differentiating characteristics that we can detect and classify.

Furthermore, the RTLS patent application proposes the usage of designed meta-atoms as a unit-cell having a geometric structure of fixed shape, in which the unit-cell has dimension smaller than a wavelength, and which may be repeated to create the metamaterial. A metamaterial may be composed of a conglomeration of one or more types of meta-atoms, but the meta-atom types can individually vary in size.

Based on this fact, the metamaterial tag in the RTLS patent is composed of a combination of subwavelength meta-atoms fabricated with copper on a flexible plastic, where each meta-atom is similar in form but with slightly perturbed dimensions so together, to either absorb or scatter different frequencies within the 77 to 81 GHz band.

Significant to the RTLS patent application, it addresses the challenge of signal interference between tags, a challenge shared among many RADAR systems.

For signal matching, the RTLS patent application uses a method to first match a tag, and its direction, and then further match the tag based on the closest time stamp.

The RTLS patent application further mentions the use of tags comprising metamaterial structures where, the tag includes a metamaterial structure, which is composed of a plurality of sub-wavelength conducting structures on a flexible dielectric substrate, in which each of the structures is tuned to resonate at a certain frequency within the band, and to which the plurality of structures as a whole will resonate at discrete frequencies within the band to create an identifying ‘spectral fingerprint’. And the resonances will respond as areas of extreme scattering, so that the anchor can detect those specific resonances for identification. Where one example of one of these structures could be a split ring resonator, which is composed of a ring with a gap incorporated, in which the structure could be interpreted (to a first approximation) as an inductor (the ring) and a capacitor (the gap) in series which creates an LC resonator having a size of the tag is less than 5 mm.

The Metamaterial structure is composed of a conductor such as copper on a flexible dielectric, and the Tag ‘spectral fingerprint’ is used to identify the user (a human person).

Also the RTLS patent application mentions that:
a) a chip less tag may have a spectral fingerprint encoded by its geometry, but not by its substance composition.
b) the chip less tag's spectral fingerprint may be identified by determining the tag's scatterers at a frequency range and then perform a FFT (Fast Fourier Transform); identifying and constructing a bandpass filter at the frequency range, which is subsequently used to window the captured IQ (in-phase and quadrature-phase) data in the time domain; averaging the data over the total number of chirped frames, to mitigate for noise, with the averaged data giving the spectral fingerprint.
c) The chip less tag spectral fingerprint may be determined by sweeping a multitude of narrowband chirps, which in conjunction provides the spectral fingerprint across the entire frequency band of operation, as follows:
   (i) setting a reduced chirp bandwidth which represents a scan for a single frequency bin;
   (ii) performing a range FFT over this reduced bandwidth;
   (iii) recording the FFT magnitude for the distance corresponding to the tag, providing the spectral response for this frequency bin;
   (iv) repeating the procedure for the next frequency bin, in which combining the data for all frequency bins provides the spectral fingerprint of the tag.

The RTLS patent applications makes use of a fingerprint matching of a passive chip less tag where a scattering profile is detected by the mm RADAR, and then converted to a binary spectral fingerprint, that is, it is a fingerprint defined as limited number of discrete frequencies for RADAR signal interrogation. A high signal magnitude of frequency response indicates for example a 1, and a low magnitude 0. Any signal response in between the frequencies does not contribute to any differentiation of the fingerprint. The mechanism can be seen as tone signaling in telecommunication systems, using a limited number of few discrete frequencies.

This means that the RTLS patent application uses a method where the chip less tag is first designed as a metamaterial having a specified scattering profile, likely fitting the binary coding algorithm, and then matched directly as a spectral fingerprint, as mentioned by the RTLS patent application method.

The chip less tag spectral fingerprint may be determined by: determining range of a scatterer from a range FFT (Fast Fourier Transform), then; identifying and constructing a bandpass filter at this range, which is subsequently used to window the captured IQ (in-phase and quadrature-phase) data in the time domain; averaging the data over the total number of chirped frames, to mitigate for noise, with the averaged data giving the spectral fingerprint.

The RTLS patent application, describes that the chip less tag's spectral fingerprint may be determined by sweeping a several narrowband chirps, which in conjunction provides the spectral fingerprint across the entire frequency band of operation, as follows:
(i) setting a reduced chirp bandwidth which represents a scan for a single frequency bin;
(ii) performing a range FFT over this reduced bandwidth;
(iii) recording the FFT magnitude for the distance corresponding to the tag, providing the spectral response for this frequency bin;
(iv) repeating the procedure for the next frequency bin, in which combining the data for all frequency bins provides the spectral fingerprint of the tag.

Previous mentioned RADAR provide a multitude of RADAR localization, measurement and sensing features but have reached limitations due to signal interference and limitations in technology chosen.

To remedy mentioned weakness, and to improve the millimeter RADAR system's performance and ability to localize, recognize, categorize, penetrate, measure, and perform remote measurement, the inventors have proposed the following RADAR system invention with collaborative and communicating target objects, without suffering from interference signals.

### SUMMARY OF INVENTION

The present invention relates to a millimeter Radio Detection and Ranging (RADAR) Signature Interrogation and Recognition (RASIR) system, and methods for recognition, classification, volume determination, remote substance analysis of target objects, especially for target objects that returns a characteristic non-linear frequency response model when interrogated with a characteristic transmitter interrogation signal; using meta-materials and other wideband chaos generating materials (WCGM) to enhance the RADAR's bandwidth and ability to recognize and classify, target objects, substances, volumes, and characteristics of target objects.

Due to the physical nature of the millimeter electromagnetic wave's behavior when interacting with different types of medium, structures substances, and material, new technical effects can be used for RADAR applications.

The RASIR system recognizes target objects by comparing non-linear frequency response patterns with characteristics of earlier sampled response patterns, stored in a Catalogue of Characteristic Frequency Response Patterns (CCFRP).

RASIR interrogates all signal interference sources available, especially those referred to as Wideband Chaos Generating Material (WCGM), by sampling each WCGM's signal response sampling model. RASIR then uses any WCGM present to improve the performance and precision when interrogating other target objects, for matching signature pattern sampling models.

WCGM transforms and widens reflected signals into a wider range of frequencies, which improves RASIR's signal detection and RADAR penetration capabilities for interrogation signals, by widening the interrogation signal's frequency band. Wideband Chaos Generating Materials (WCGM) are typically based on a metamaterial structure configured to reflect, transform, and widen signal frequencies in the millimeter RADAR band into signals having an extended bandwidth with sub-frequencies having wider wavelengths.

The RASIR system can locate, determine a position of, and interrogate any Wideband Chaos Generating Material found, to capture its complex RADAR signature model and behavior. The RASIR system may use detected Wideband Chaos Generating materials as remote slave interrogation signal generators and transmitters for improved interrogation analysis precision, localization, and recognition of other target objects.

It is an objective of the present invention to accomplish a millimeter Radio Detection and Ranging (RADAR) Signature Interrogation and Recognition system, further denoted RASIR 1, by introducing methods for interrogation, classification, and localization of target objects having characteristic frequency dependent frequency response sampling model, especially non-linear frequency dependent frequency response sampling models. Precision in angle and range detection are further enhanced using neighboring interference signal generating objects, further denoted as Wideband Chaos Generating Material (WCGM) 30. The Wideband Chaos Generating Material is usually composed of a designed metamaterial with the ability to transform a RADAR signal into a new frequency spectrum having components of both higher frequencies with shorter wavelengths, and lower frequencies with longer wavelength before reflecting the signal. This also increase the interrogation frequency bandwidth, with the wavelengths that enables detection of target objects that otherwise would be ambiguously detected from millimeter RADAR wavelengths, meaning that the object is detected with better resolution, and that smaller size of objects can be detected. Natural occurring objects and artificial objects with WCGM frequency response and transformation abilities, located in the proximity of a target object may be discovered by RASIR, interrogated, and identified as WCGM; and then further used as WCGM to enhance RASIR's ability to interrogate and locate target objects.

### Technical Problem

A technical problem and challenge is to make and configure a new multi-purpose millimeter RADAR system, and to make it able to improve previous millimeter RADAR system's performance and to improve the ability to localize, recognize, categorize, penetrate, measure, and perform remote measurement (including non-invasive measurements in the human body) of known and mapped recognizable target objects, including target objects having non-linear frequency dependent response signals and target objects generating chaotic wide-band interference signals.

It is recognized that earlier mentioned millimeter RADAR system inventions (mentioned under the heading Background) seems to suffer from:
a) degraded SNR due to interfering signals resulting from interference generating objects such as what later in the invention presented here will be referred to as Wideband Chaos Generating Material (WCGM) and Meta Material Interference Objects (MMIO) 31 and Natural Objects (NO) 32 having the ability to alter frequency, phase and attenuation of reflected RADAR signals;
b) rigidness in fingerprint detection of target object's radar signatures are analyzed in the frequency domain, where discrete samples of sub-band frequencies establishes a binary zero or one digit in an identity code, using an FMCW millimeter RADAR;
c) inability to perform non-invasive millimeter RADAR inspections of a human or animal body;
d) a need of active tags to remotely signal a state of a target object, or RADAR tag, requiring active electronics and wireless RFID, Bluetooth and Wi-Fi signaling;
e) RADAR tags that needs to be based on electronic circuits, let alone battery less passive resonance circuits, in the form of specially designed metamaterial having a specific impedance pattern designed for mentioned binary decoding;
f) a need for better target object localization and distance mapping precision;
g) a limited means to detect target objects hidden behind dominant responses from primary target objects being detected;
h) a lack of ability to categorize formerly unknown newly detected target objects;
i) missing means to calibrate a 3D environment and to mark invisible borders, paths and object locations for mobile robots and logistic solutions; and
k) limited abilities for RADAR tags to act as remote sensors, readable by the RADAR

The technical problems addressed by the RASIR system and methods are how to:
a) turn normally deteriorating interference signals from Wideband Chaos Generating Material, into useful signal resources for target object interrogation, categorization, and improved recognition, location and mapping;
b) reach and interrogate target objects, fluids and internal organs of the human bodies that are absorbing, or shielded from, millimeter RADAR signals;
c) achieve a robust, adaptive and trainable method for categorization and recognition of target object's receiver signature, when interrogated by a RADAR transmitter's interrogation signal having one, or several frequency components, or a specific Chirp waveform;
d) to remotely retrieve physical and chemical sensor signals from a distance using a millimeter RADAR, also in combination with a millimeter RADAR tag; and
e) to provide a lifecycle sustainable low cost millimeter RADAR tag with distance invariant and/or angle invariant uniquely determinable RADAR frequency response characteristics for object identification, location tracking and remote sensing.

Hence, a RADAR system, denoted RASIR, has been created, composed of a RADAR system and RASIR:s function for recognition, localization and mapping of collaborative and communicating target objects, as well as natural objects, meta material, and objects having non-linear frequency dependent response signals; without suffering from interference signals, as follows.

### Solution to Problem(s)

An interesting technical effect of the RASIR system is its ability to perform localization, distance measurements, sensing, tracking, pattern matching of non-linear frequency dependent reflections from target objects, especially under influence of special target objects natural or designed acting as RASIR's so called Wideband Chaos Generating Material (WCGM).

For narrowband signals in RADAR systems, the range resolution is low, which is usually insufficient to divide the target into multiple extension units. There is no phenomenon of incoherence between the scattering points of the target, which means that the chirp of the scattering points of the target is processed by coherent accumulation, i.e., it is accumulated before the envelope detection.

Increasing the band of signals in detection of radar targets means increasing the chirp accumulation which in its turn results to improve the performance of weak target detection. By widening the signals band, higher resolution range is achieved which can usually distinguish the target into multiple range units in the range dimensions. The phenomenon of incoherence occurs between the chirps of each range unit. Generally, only incoherent accumulation can be carried out, i.e., accumulation can be carried out after the envelope detection, so the phase information of the chirp is lost.

Thus, the target chirp of a wider band radar is usually superimposed by the chirp of multiple scattering range units from the target, which enables it to have a better suppression ability of target Radar Cross-Section (RCS) fluctuation. Meanwhile, the incoherent accumulation loss of the chirps from multiple range units of the target is also a key factor affecting the detection performance of the RADAR. If the scattering characteristics of the target are predicted in advance and matched waveform signals are transmitted or filtered by matched filters, the same detection performance as narrowband signals can be achieved based on high resolution. However, most of the target scattering characteristics of the current state of art of the wideband radars are unknown and difficult to obtain, so only incoherent accumulation can be carried out. Although incoherent accumulation brings additional signal to noise ratio (SNR) loss, it also inhibits the fluctuation loss, and the detection performance of the target is the synthesis of these two factors. Under the influence of the balance of the two factors, the optimal bandwidth is obtained.

The Wideband Chaos Generating Material (WCGM) is usually composed of a designed metamaterial with the ability to transform a RADAR signal into a new frequency spectrum having longer and lower wavelengths before reflecting the signal. Also, the WCGM causes multiple signals with possible phase shift in relation to the RADAR signal generated. It is also possible to use designed metamaterial interference objects (MMIO) and Natural Objects (NO) instead of a WCGM, to generate a wider bandwidth, and/or phase variations.

Using the WCGM in conjunction of a wideband radar system principally a wider band of signals in detection of radar targets is achieved and the generated signals by WCGM have phase variations in relation to the radar signal. As far as the WCGM is a designed device, the scattering characteristics of it is known which is affecting the scattering characteristics of target.

In using the WCGM in conjunction of a wideband radar system, the following improvements and technical effects are made possible.

The WCGM contributes to increasing the band width of signals:
a) The chirps accumulation is increased which in its turn results to improve the performance of weak target detection,
b) The target chirp is superimposed by the chirp of multiple scattering range units of the target, which enables it to have a better suppression ability of target Radar Cross-Section (RCS) fluctuation,
c) The incoherent accumulation of the chirps from multiple range units of the target is also an important factor affecting positioning resolution.
d) The incoherent accumulation brings additional strength to inhibits the fluctuation loss which enables to have a better suppression ability of target Radar Cross-Section (RCS) fluctuation, i.e., smaller target or smaller RCS can be detected.

According to a second aspect the generated signals emitted from WCGM which have phase variations, the lost phase information of the chirp using only the radar system is compensated by another sort of phase information. It is notable that the lost phase information is related to the chirp from multiple range units of the target. The new phase information is related to phase variational signals from WCGM which are affecting the multiple range units of the target, which is as several target scanning with different positioning of RADARs.

According to known scattering characteristics of WCGM the scattering characteristics of target is affected. Using certain target (such TO 22, and especially the passive tags TODS 25, TOwNLF 26, STO 27), the similarity of combination of scattering characteristics of target and scattering characteristics of WCGM 30 in different target position is significantly increased which results to better and more stable target detection.

According to combination of WCGM signal and RADAR signal, the incoherent accumulation of radar signal which brings SNR loss, is compensated and SNR is significantly increased which results to better target detection and better range of scanning.

The RASIR system solves the technical problems, while making use of the technical effects mentioned, as follows.

Instead of facing interference signals from Wideband Chaos Generating Material (WCGM) and reflections as deteriorating effect for a RADAR system; RASIR views interference signals as and Wideband Chaos Generating Material as a useful resource to extend its transmitter frequency bandwidth and to generate frequencies of wider range of wavelengths than the millimeter frequency band, for the RADAR transmitter; and as remote signal sources for signal triangulation. Although not necessarily the first step, the system may first map the location of a Wideband Chaos Generating Material (WCGM) 30, or a similar object such as a Metamaterial Interference Object (MMIO), or a Natural object having metamaterial properties (NO).

The WCGM, MMIO and NO acts as objects causing reflections, signal interferences and even delayed signals with altered frequencies, phase shift, doppler shift, polarization, or as combinations. In most RADAR applications such objects would be seen as problematic, but not in the RASIR system, as RASIR makes use of its knowledge about how radio and RADAR waves are reflected and transformed by these objects.

When the RADAR function has located the WCGM, or an interference object such as MMIO and NO, it may interrogate the WCGM using a transmitter interrogation signal (TIS) 7, to receive response from the WCGM in the form of an interrogation receiver signature (IRS) 9. By then analyzing several TIS and RIS over multiple frequency sub-band windows, a pattern matching function (PMF) may recognize and categorize the RADAR signature and generated response signals, as well as underlaying response signal functions. This categorization may then be looked up, and if found new and unique, then the new pattern with categorization is stored in a Catalogue of Characteristic Frequency Response Patterns (CCFRP) 12.

Once RASIR as recognized and located a WCGM, it may then start to make use of the WCGM as a slave Altered Transmitter Interrogation Signal (ATIS) 36 transmitter, that means that the WCGM can be used as an interrogation signal generator. RASIR first transmits a previously known TIS 7 signal directed towards the WCGM. RASIR detectable target objects comes in many forms such as Target Object Inside Body (TOIB), that is organs with, and volumes comprising water-based solutions in the human stomach, intestines, lungs, and blood vessels, and other organs and volumes inside the human body, or any organism.

The WCGM then scatters its transformed and reflected ATIS 36 signal towards any of the RASIR target objects 22-29 (TO, ATO, . . . TOIB); which then reflects and scatter its IRS signal towards a receiver Rx Ant 18, where both the IRS signal and the related Altered Transmitter Interrogation Signal (ATIS) signal which is returned from the (WCGM, MMIO, NO) are propagated back to the RADAR via at least one RADAR Rx Antenna 18, and Receiver Rx 19.

Continuous Tx signals 12 are transmitted by the Tx Antenna 17 while receiving via Rx Ant 18 mentioned reflected and transformed returned signals from the target object (TO) 22 and other objects 23-29, together with reflected and transformed signals from the WCGM, MMIO, and NO. To process all signals transmitted based on the TIS and received later defining the IRS, all signals are mixed in a Tx Rx Mixer 20, where all signals are captured while being frequency and time correlated. In application using direction sensitive antennas having single or multiple Rx and Tx channels, RASIR can identify the direction of a received signal, using a Target Object Position and Time of Flight estimation function (TO POS TOF) 21 that may act as a direction sensitive RADAR, allowing early direction mapping of target objects TO 22 of any kind or sub class 23-32. Analysis of relative target object location and direction may also be deferred to later analysis functions, and after FFT analysis 8 and similar signal processing and as a step within the pattern matching function PMF 10. Hence, in its simplest form the RASIR RADAR function may use a single non-direction sensitive transmitter and receiver antenna without any direction sensitivity. In more complex configurations RASIR may be equipped with more antenna elements that may transmit signals in different directions, while also having multiple receiver antennas able to pick up the direction of a certain received signal.

Transmitted Tx signals can be mixed with received Rx signals in a Tx Rx Mixer 20. To ease pattern matching of recognizable signals RASIR may process these signals to mathematically more convenient formats, for example using FFT 8 to transform sampled signals into the frequency domain for further pattern matching in the Pattern Matching Function (PMF) 10.

Characteristic patterns for matching are stored in the CCFRP database as RTIS and RIRS pairs; which corresponds to reflections from recognizable individual target objects 22-32 being interrogated using a TIS 7 signal, while reflecting an IRS 9 signal.

As signals are translated from time domain into the frequency domain, signal pattern recognition and matching can be based on matching frequency response curves between transmitted TIS and received IRS signal representations; and earlier recognized unique RTIS 37 and RIRS 38 signal representations in CCFRP 12. Of course, RASIR is not limited to pattern matching in the frequency domain but may use any type of signal pattern matching method, even using artificial intelligence methods such as neural networks, convolutional neural networks, and more advanced pattern matching applications. Also, the representation of the Pattern Matching Function (PMF) 10, and CCFRS 12 database of recognizable patterns may be implemented as a trainable artificial neural network, such as a deep learning neural network in learning mode.

RASIR can make use of the Pattern matching function PMF 10 to map a characteristic signal response from an individual WCGM 30, when interrogated using a TIS 7 signal, resulting in a received IRS 9 signal. See FIG. 2a, and 2b. Once the WCGM reflected 44, 50 signal response is recognized, RASIR may start to make use of the WCGM generated Interference Frequency Pulse Reused 51 as a slave remote RADAR signal source 51, enabled by RASIR's WCGM awareness. When a Target Object 22-29 receives an Interference Frequency Pulse Reused 51 from WCGM, MMIO, or NO; which all shares similar RADAR signal reflecting behaviors; then the TO will reflect this interference reflection pulse and frequency signature 52, back to RASIR's 1, receiving RADAR 2 Rx antenna 19. See FIG. 2b. This allows RASIR to interrogate, using an Altered Transmitter Interrogation Signal (ATIS) 36, other target objects TO 22-29 using WCGM 30, MMIO 31, NO 32 as means to generate characteristic local interrogation signals. As a result of this effect, RASIR can turn otherwise unwanted interference signals into useful information sources having characteristic recognizable and useful signal characteristics for interrogation of TO:s 22-29 using new interrogation frequency patterns not available at the RASIR RADAR transmitter Tx, and where the WCGM, MMIO, and NO may have a preferable location for interrogation of TO:s 22-29.

Pattern matching is made by comparing the target object's (TO) frequency response signal signature in the frequency domain for best signature sampling model, or signature model correlation or similarity (10′) with reference interrogation receiver signatures (RIRS). PMF 10 may also perform a similar recognition of a target object's TIS 7, that is defining the Tx 33 signal, as well as any Altered Transmitter Interrogation Signal (ATIS) 36 transformed by the Wideband Chaos Generating Material (WCGM) 30, or similar materials MMIO 31, and Natural Objects 32, sharing similar meta material behavior, that is a determinable preferably non-linear frequency dependent RADAR reflection and transformation.

Hence, RASIR may make use of WCGM s as extra slave signal sources for interrogation of target objects. Other signals paths for this Altered Target Interrogation Signal (ATIS) 36, 35 are possible for other interrogation scenarios involving WCGM 30, feasible natural objects (NO) 32 and metamaterial interference objects (MMIO) 31. As WCGM's location is known, it may also support triangulation of a target object.

This solves technical problem a).

RASIR can be used for non-invasive body examination, using a Wideband Chaos Generating Material (WCGM). The WCGM is then transforming the TIS signal into a wider frequency spectrum ATIS with abilities to penetrate and interrogate, body fluids, organs, a tag, a probe, and natural material inside a human body. In the actual case the target object, is referred to as a Target Object Inside a Body (TOIB), that is a section or volume of body tissue.

This solves technical problem b).

RASIR uses its Pattern Matching Function (PMF) 10 for signal signature sampling model matching of returned IRS signatures in the frequency domain. The IRS relates to a specific TIS, which can be seen as an input and output signal pair defining the target object's signal signature sampling model. If the PMF cannot find a corresponding reference IRS (RIRS) and corresponding reference TIS (RTIS), then in case RASIR is set in a learning mode 60, then the PMF are storing the new pair of TIS and IRS, in CCFRP 12, as a new RTIS and RIRS signal signature sampling model.

Prior art solutions have used fingerprint decoding of IRS at specific wavelengths using multiple bandpass filters, to extract a binary number based on amplitude or intensity of certain distinct frequencies in IRS. Such a solution has drawbacks such as being rigid locked to specific frequencies, prone to signal interferences, does not detect Doppler shift due to target object's radial velocity towards and away from the RADAR Tx and Rx antennas, it may not accept natural objects and new types of target objects having IRS with new frequency fingerprints, and the function is sensitive for variations in signal intensity which may highly occur due to the distance dependency of RADAR interrogation.

RASIR's PMF on the other hand is doing pattern matching of a signal signature sampling model alignment, independent of its signal intensity, using pattern matching and alignment methods such as, least mean square methods and sophisticated matching methods. This results in a robust pattern matching function that may cope with new situations and new target object types and interrogation TIS and IRS pairs. As the PMF matches current TIS and IRS pairs with earlier reference pairs RTIS 37 and RIRS 38 in CCFRP 10, for best signal signature sampling model match, RASIR may find and handle several matching candidates in CCFRP. RASIR may also be configured to keep track of the location of several identical objects at different locations by storing data records in the CCFRP database function.

If a target object is not previously registered in CCFRP, and RASIR is set in learning mode, it may record and trace new target object's characteristic TIS and IRS pairs, and target object location and movement vectors. As a target object's characteristic TIS and IRS response is properly recognized, it may be used as a base for incrementally calculating a better signal fit for RADAR distance, direction, and velocity mapping. If a best match in CCFRP cannot be determined, CCFRP may propose alternative RTIS signals to be used for the next transmission of a new transmitter interrogation signal (TIS), optimized to generate the best differentiating IRS, based on previously known RTIS and RIRS pairs in CCFRP, and earlier interrogation signals for the target object being interrogated.

A new TIS may involve usage of narrowband frequencies, Chirps of different shapes (triangular, square, pyramid, falling, exponential, hyperbolic, sinusoidal to mention a few), Frequency Hopping Strategies, and a narrowband window with a known distribution of frequencies.

This solves technical problem c).

As RASIR provides tags consisting of substance demonstrating non-linear frequency dependent millimeter RADAR response sampling models, where the sampling models may be altered, or the tag itself hidden from the RADAR, these RADAR tags may be used as remotely readable RADAR tags with sensor functions. The following target object solutions in RASIR may all act as remote sensors beyond signaling a relative time of flight distance, angle location, characteristic TIS and IRS pairs, velocity vector, and identity if recognized in CCFRP.

| | |
|---|---|
| ATO | Active Target Object 23 can transfer information by altering its frequency |
| | response due to a physical input such as: temperature change, rotation of an object, or by |
| | exposing and hiding a target object for RADAR interrogation. |
| LTO | Location Target Object 24, such as a measurement probe for measuring objects where target |
| | objects as hidden and made visible to the RADAR, as a mean to signal information. Robotic |
| | and other vehicles may use this technique to electronically signal a flashing target object as a |
| | reflector carrying a certain time dependent signature. |
| TODS | Target Object Detecting Solutions 25, such as a variable water sugar concentration, which |
| | may alter its IRS due to a change in water concentration in a water salt, sugar, sucrose, or |
| | carbohydrate solution, gel, or a chamber. |
| HFD Diaper | High Frequency Detection Diaper 28, for remotely measuring water and body fluids in |
| | proximity of human body and in contact with human skin. A special variant of HFD Diaper, |
| | is as a wound dressing having a similar body fluid absorbing function. |
| TOIB | Target Object Inside Body 29, for measurement of water and body fluids inside the human |
| | body. |
| MMIO | Metamaterial Interference Object 31, that may consist of a frequency shifting and harmonic |
| | metamaterial having a tuned impedance, or resonance circuit, which may be turned on and |
| | off. |

Hence technical problem d) is also addressed and at least partially solved.

As a Target Object with Non-Linear Frequency dependent response signal (TOwNLF) 26, can be produced using a sugar-water solution, and a millimeter RADAR invisible coating to prevent evaporation of water content, such a RADAR tag would even be digestible.

But the TOwNLF tag may as well be produced based on any other substance demonstrating a non-linear frequency response signal not limited to carbohydrates, salt, proteins, dissolved or in combination with water or other fluid solution, gel form, mixed with paint, in fluid or solid form.

In matter of fact, nature may offer biological material that may be used as temporary objects for tracking. For example, a grape, a leaf, a growing plant, and food binding water molecules may demonstrate a recognizable non-linear frequency response signal model and may all be used as temporary or permanent detectable target objects. Typically, a sugar-water concentration solution-based substance, fruit, object would provide a distinct IRS for each ITS reflected.

Hence the RASIR system provides multiple solutions to technical problem e) as target objects may even be grown as a plant or produced by natural biodegradable and even digestible substances.

### Advantageous Effects of Invention

In the most general terms RASIR is a millimeter RADAR system able to distinguish, determine, recognize, and categorize target objects based on target object's frequency response signatures, by matching interrogation response signature IRS sampling model alignment against earlier classified target object's reference interrogation response signatures (RIRS), and know-how about target object characteristics. The pattern matching function (PMF), searches for a matching RTIS and RIRS pair by matching similarities in at least one of the following dimensions: frequency domain magnitude sampling model matching; time domain signal sampling model matching, doppler shift sampling model matching, and matching of polarization signal characteristics.

Target objects may be any type of object reflecting, or returning millimeter, or centimeter wavelength RADAR signals, called Interrogation Receiver Signature (IRS). RASIR is specially configured to determine and recognize any non-linear frequency dependent signal response IRS, returned from a target object after having received a signal generated according to RASIR's specific transmitter interrogation signal (TIS). It is an advantage that RASIR can interrogate both linear and non-linear frequency dependent signatures as well as time delayed resonance signals from target objects having an ability act as a resonance circuit, due to its impedance, its intrinsic resonance electric circuit model, and tuned frequency used in the TIS specified RADAR signal.

A distinguishing factor in RASIR is that it can recognize and categorize signature sampling models based on multiple sampled unique TIS and IRS pairs, not just as repetitions of same TIS.

RASIR may also use multiple TIS:es (7) for interrogating using TIS in the form of multiple narrow sub-band frequencies, alternatively as a selected Chirp type. Chirps may have a frequency scan that is raising, falling, square formed, triangle hat formed, sinusoidal distribution of frequency intensity over time, or an exponential frequency profile, over a short time window. By varying the Chips and TIS, RASIR's Pattern Matching Function (PMF) can match multiple dimensions of a target object's signal signature characteristics and IRS:es (9), to determine its similarities with earlier known, or newly discovered IRS characteristics.

The RASIR's PMF uses a Catalogue of Characteristic Frequency Response Patterns (CCFRP), implemented as a database function holding records of earlier matched Reference Transmitter Interrogation Signals (RTIS), and Reference Interrogation Response Signatures (RIRS). By letting the PMF compare and match actual TIS-IRS with previous RTIS-RIRS pairs, and to return an index reference to at least one data record in CCFRP, in the case where at least one match may be identified; the RASIR system offers information access to knowledge about the target object's category, related match, or matches in CCFRP for at least a first and potential second-best match. CCFRP may then provide information about the TO that was not previously known, to further improve how to interrogate the TO for improved classification and matching precision, and to provide a TIS RADAR signal that would generate the best IRS, in search for a specific Target Object (TO), by using a Time-of-Flight (TOF) RADAR analysis method, and a RADAR antenna angle to Target Object calculation. RASIR can also optimize the precision by selecting the most preferred TIS that would generate the most distinguishing IRS. Alternatively, RASIR may generate multiple TIS signals, that potentially results in the best distinguishing IRS data: for doppler RADAR calculations; to interrogate signal reflected magnitude; to interrogate signal phase shifts; to interrogate polarization for matching and signal sample recognition of the target object's signal characteristics. Mentioned method is also applicable in a real scenario where each target object's environment may comprise multiple alternative and competing target objects.

An alternative implementation of the CCFRP and PMF is to make use of a neural network function, based on a configured pattern matching function in the form of a Convolutional Neural Network (CNN) configured for signal sampling model matching of TIS-IRS and RTIS-RIRS pairs in CCFRP. The CNN may optionally be enhanced by a Deep Learning Neural Network (DLNN) to allow for discovery, classification of new TO:s having unique TIS, and IRS pairs analyzed and found to have a distinguishing new TIS to IRS signature sampling model.

An analogy for a sound-based technique, would be to search for a wine glass of a certain resonance among others by interrogating the space using a few ITS sound signals of matching multiples of the resonance frequency. Received resonance signal sampling models, analogue to IRS:s would then prove existence of a resonating wine glass. By then selecting a non-resonating frequency, the corresponding IRS would contain signal components reflecting other objects but no resonance signal from the wine glass's ego resonance frequency.

As a result, RASIR can determine existence of a unique TO having a unique TIS and IRS characteristics, especially when the IRS are of non-linear frequency dependent signature character resulting in a return signal of a magnitude, even among other TO:s having a linear frequency dependent signature not following the same frequency IRS magnitude sampling model. Examples of TO materials having a non-linear frequency dependent TIS to IRS characteristics are, salt-water solutions demonstrating a second-degree polynomial equation function model, and sugar-water solutions demonstrating a third-degree polynomial equation frequency dependent response magnitude function model. Also, different sugar-water concentrations, denoted as Brix grade values, demonstrates variations in characteristic sampling models. Metallic and conductive materials, usually have a linear frequency dependent response sampling model, of the shape of a first-degree equation shape, possibly limited by intrinsic antenna lengths in the material. Hence, it is an advantage that RASIR can determine TO material composition, as well as water contents in a sugar, salt, or combined solution. More materials exist that demonstrates unique TIS to IRS sampling models in the frequency domain with magnitudes, such as starch, carbon hydrates, and other natural occurring substances, water-based having di-pole effects, and organic materials with water content. A sub class of target objects, named Wideband Chaos Generating Material (WCGM), with its own sub-class named “metamaterials” are all demonstrating characteristic RIS to IRS signal sampling models. WCGM and metamaterials can be customized, designed, and configured to demonstrate a characteristic frequency response signature function model, according to a predefined specification.

In relation to prior art, RASIR can discover new unique TIS-IRS differentiating target objects and new material compositions where a non-linear dependent frequency to magnitude pattern in TIS-to-IRS can be distinguished from a linear dependency. RASIR may also recognize signal resonances sampled in the IRS, in relation to corresponding TIS.

Another advantage is that RASIR can recognize IRS containing frequency transformations due to harmonic behavior, resonance, interference, and combinations of these signal components in an IRS. As RASIR may interrogate a target object of any kind isolated in a volume in a MIMO antenna configuration, or distance segment in SISO antenna configuration; it may also interrogate the target objects complete response spectrum and resonance characteristics, to learn how the object would respond to a future characteristic TIS signal, that is which IRS that could be expected from a given TIS. This capability to interrogate and map target objects as well as Wideband Chaos Generating Materials (WCGM), a sub-class of target objects, is especially useful as will be further explained.

Most RADAR, and millimeter RADAR systems struggles with signal interferences from other sources and reflections than the actual interrogated target object. Such interference signal sources can generally be viewed as background disturbances reducing the detection ability of the actual target object being interrogated. Therefore, any occurrences of a Wideband Chaos Generating Material (WCGM), generating interference signals, would normally be seen as problematic for traditional millimeter RADAR system. RASIR challenges this perspective by turning this problem into an opportunity, making use of extra signal information available in from a reflected known signal from a WCGM, and then reflected by a target object under interrogation. For example, the WCGM may create an opportunity to interrogate target objects using the wideband frequencies generated by a WCGM reflecting narrowband RADAR signal or chirps; which then reaches a target object under interrogation by RASIR.

In RASIR all signal sources are seen as potential signal sources that may contribute as secondary transmitter interrogation signal data, generated as a secondary effect of the transformation of a TIS to IRS in a feasible object, here denoted as Wideband Chaos Generating Material (WCGM). The WCGM is a subset of other target objects but having a characteristic useful ability to reflect wider band signals, resonate with signals, and to create signal interference and frequency transitions in a response signal. WCGM may be designed as manmade metamaterial comprising an array of resonance circuits tuned to a receiving frequency, and able to emit a signal at another wavelength to at least some degree. The same WCGM may also receive signals on an alternative frequency and transform the frequency into a by the radar readable wavelength, preferably in the millimeter wavelength, but it may extend to wider range of wavelengths such as into the centimeter RADAR bands.

An analogue model using sounds, is to send signal at resonance signal into a room with a resonating wine glass. The wine glass will generate a characteristic signal resonance signal. This signal makes a singing sound that may be used to interrogate other objects with similar resonance behavior in the proximity to the resonating wine glass.

The following scenario is presented in FIG. 11f “Radio wave SISO with WGCM to Target Object (TO) forwarding”.

Preferably RASIR is first interrogating its WCGM using a TIS and by analyzing its IRS using its sampling model matching function (PMF) to identify the matching frequency response model for the WCGM; and then storing the response sampling model comprising pairs of TIS and IRS from the WCGM interrogation in CCFRP, unless previously interrogated and mapped.

In this scenario, RASIR first transmits a TIS signal towards a target object in the form of a Wideband Chaos Generating Material (WCGM), reflects its IRS. This IRS signal from the WCGM is then acting as an Altered Transmitter Interrogation Signature (ATIS) 36, for interrogating a second target object TO, subject for final signal interrogation, by RASIR. The ATIS from WCGM is then reaching the secondary TO, which returns its IRS back to the RASIR Rx Receiver antenna, for further analysis and processing in the PMF and CCFRP. PMF is then performing a sampling model matching of the second TO's IRS because of the WCGM's ATIS signal, for response signature sampling model matching.

$${IRS}_{WCGM} = {WCGM}_{TIS - to - IRS} ( {TIS}_{Tx - from - RASIR} ( TISG ( ) ) )$$

RASIR is then interrogating the second TO using the signal combinations where IRS_{WCGM}, also is referred to as ATIS generated by the WCGM of:

$${IRS}_{TO} = {TO}_{TIS - to - IRS} ( {TIS}_{Tx - from\_RASIR} + {IRS}_{WCGM )}$$

where (IRS_{TO}+IRS_{WCGM}) reaches the RASIR Rx Receiver to be captured as an IRS signature in RASIR for sampling model matching analysis in PMF using CCFRP.

PMF may then extract and remove the IRS_{WCGM} signal component from the IRS in RASIR to further identify frequency components and characteristics signal signature of the IRS_{TO} signal, when interrogated with the IRS_{TO} being used as a TIS generated locally by the Wideband Chaos Generating Material (WCGM) to interrogate the second target object.

Hence, RASIR can make use of interference signals from WCGM in near proximity to a target object for distance interrogation.

By using an Wideband Chaos Generating Material (WCGM), specially designed metamaterial or discovered natural object (NO) having feasible signal transformation properties, a RADAR transmitter interrogation signal (TIS) 7 can be transformed into a new Altered Transmitter Interrogation Signal (ATIS) 36 having a new frequency spectrum and frequency components that may penetrate thru material and tissue otherwise not reachable by a millimeter RADAR TIS signal, such as Shielded Target Objects (STO) 27, and Target Objects Inside (a human) Body (TOIB) 29 protected by a skin layer.

Multiple signal paths and scenarios are presented in FIG. 11a-g, where the last FIG. 11g describes a situation where a signal is transferred from RASIR Tx to WCGM to and then back to WCGM further on to RASIR Rx, as a major signal path. Other signal paths are also open and will be combined and will be sampled into a RASIR IRS signal. The RASIR IRS signal may be captured as a timed sample defined by time quotas for sampling. As an alternative the RASIR IRS signal may be representing a rolling sample window of a determined or adjustable size.

Advantages of using at least one WCGM to reach at least one TO are:
i) Ability to reach otherwise Shielded Target Objects (STO), that could not otherwise be directly interrogated by RASIR's RADAR Tx and Rx antennas.
ii) RASIR's ability to translate a millimeter RADAR frequency to wider wavelengths able to penetrate the human body for Target Objects Inside Body (TOIB), for non-invasive medical examinations; and also as a mean to reach thru surfaces that reflects or absorbs millimeter RADAR wavelengths.
iii) To reach and to interrogate a High Frequency Detectable Diaper's (HFD Diaper) fluid compartment, one may sue an Wideband Chaos Generating Material (WCGM) that translates millimeter wavelengths to slightly wider wavelengths, able to reach thru textile, plastic sheet, and clothes.
iv) That once the WCGM signal translation characteristics and its location is known, is becomes possible to triangulate the location of target objects using yet another signal source.
v) Once the WCGM is identified, it can be pinged separately toe generate a local TIS for further interrogation of target objects. A variant of the WCGM may be equipped with an electronic switch 831 that makes it possible to turn it on, off, and in some situations even adjust the signal characteristics of the WCGM, which further increase the possibilities to perform more advanced measurements of Target Objects (TO).
vi) As soon the signal transformation behavior of one WCGM has been fully discovered, that is its TIS to IRS sampling model, analyzed in PMF and stored in CCFRP; RASIR is then able to make use of the WCGM for further analysis and mapping of new target objects near said first WCGM.

Interrogated target objects are matched with previously recognized reference frequency domain-based RADAR signatures, by comparing sampling model alignment of received Interrogation Response Signature (IRS) from a target object, with earlier stored Reference Interrogation Response Signatures (RIRS), stored in a Catalogue of Characteristic Frequency Response Patterns (CCFRP). Recognized signal sampling models are then indexed in the CCFRP, to offer access to reference signal data and object classification data. New target objects recognized, may be stored in CCFRP as new RIRS if the system is set in a learning mode 60, see FIG. 10. Then the target object, and similar target objects sharing the IRS to RIRS sampling model match may be recognized and found in CCFRP for further, reference, indexing, use and improvement of localization, categorization, and tracking. Hence, a matched IRS to RIRS only need to refer to a data record in CCDRS holding a RTIS and RIRS pair, and preferably also holding more information such as RASIR Tx and Rx Antenna relative position of found target object items, for tracking of objects, and preferably more data record information such as: last update time and date, identity of object, classification of object, category of object type, object state, indicated Brix degree value in case a target object represents a sugar-solution just to mention a few useful information elements.

The RASIR system interacts with different types of target objects 22, with RADAR signature features that are: reflecting RADAR signals; switching polarization; switching the phase of a wave, transforming frequencies; generating doppler shift for objects having a velocity; distorting signal waveforms; adding or subtracting new signals generated as signal interference; contributing with signal resonance components and delayed frequency response sampling models; transforming TIS wavelengths to wider wavelengths to penetrate objects blocking millimeter RADAR, thus creating an Altered Transmitter Interrogation Signal (ATIS); and in combinations.

Some of the more specialized target objects demonstrates a reflected non-linear frequency dependent response signature, meaning that the object returns a characteristic non-linear frequency sampling model, or fingerprint, having a deterministic signal distribution and intensity over a frequency range; when interrogated with a TIS having just a narrow frequency band, swept over a sub-band, or using a waveform of a Chirp signal, transmitting a frequency scanning TIS.

This lets RASIR interrogate TO:s using multiple narrow frequency sub-band TIS, to capture corresponding IRS pairs; and then not just match alignment of TIS and IRS signature sampling models with RTIS and RIRS pairs stored in CCFRP, but also to make several matching procedures for each sampling model matching window 202, 212, followed by more sub-band windows for matching beyond 202, 212 if needed to ensure unique and reliable sampling model matching.

Other target objects can alter their intrinsic frequency response sampling model due to changes in physical, chemical, structural, and electronic properties, and states. An example of a physical changing object is the Active Target Object (ATO) 23 which can transfer information to the RADAR by altering its frequency response due to a physical input such as: temperature change, rotation of an object; or by exposing and hiding a target object for RADAR interrogation and used in the position measurement probe referred to as the Location Target Object (LTO) 24. The LTO lets a user measure and collect geometrical locations relative to the RADAR set up, by signaling a position and time for recording to the RASIR system, a procedure necessary when calibrating a RASIR system at a certain location, but also useful when recording object locations and boundaries that cannot distinctly be recorded as target objects themselves. Example applications for the LTO is to define invisible walls, physical floor areas for logistics robots, roads, paths, safety zones, indoors and outdoors; for definition of navigation restrictions in a 2D or 3D world. The LTO may also be adding a time stamp for the recording event and 3D position being measured.

Yet another example of a special target object is the Target Object Detecting Solutions (TODS), which provides characteristic recognizable frequency sampling models for different concentrations in a water-based solution in for example a sugar solution, a carbohydrate solution, a salt solution. This is further specialized when applied onto a target object in the form of a High Frequency Detection (HFD) Diaper with a RASIR based system for measurement, alarming, logistics handling, and human care; where the HFD Diaper's frequency dependent signal characteristic changes when water-based substances such as urine, blood, body fluids and feces is received in a receptacle or moisture receiving substance of the HFD Diaper. The RASIR system can detect changes in RADAR signature by response sampling model matching to indicate a certain state of the HFD Diaper, thus letting the HFD Diaper act as remote sensor, readable by the RASIR system. The HFD Diaper design may also be made in a form for wound dressings collecting and monitoring blood, pus and plasma altering its frequency response.

RASIR may detect and measure Target Objects Inside of a human Body (TOIB) for non-invasive measurements. The RASIR system may emit and measure mm Wave RADAR signals passing through a living human or animal body, or reflected from the human body, where internal organs, body substances, volumes, and body fluids are measured as target object TOIB. When the TOIB receives a mm Wave burst having a feasible wavelength, these waves may penetrate thru the body, allowing RASIR to measure RADAR signals passing thru the body, or reflected by the body in the skin layer or deeper, or using a combination. The TOIB are volumes suited for non-invasive body measurement. When the TOIB receives a RADAR signal, such as a TIS or ATIS; the TOIB then transforms, reflects, returns, and/or emits a frequency response signature that corresponds to the character of the TOIB and its physical state, determinable from its RADAR signature sampling model. For example, a stomach may have a certain signature when empty and another when filled with fluid, or food.

Physical properties of the Target Object Inside Body (TOIB) that influences its RADAR mm Wave signature response are changes in body fluid composition and concentration, especially when based on water molecules demonstrating a di-pole effect. The water molecule di-pole effect results in different resonance frequencies creating a resonance phenomenon at certain RADAR frequencies, and different attenuation at different frequencies. As higher than 120 GHz millimeter RADARs does not penetrates the human body deeper than into the upper layers of the skin, a non-invasive body measuring TOIB application, will have to adapt its RADAR frequency using longer wavelength RADAR and lower frequencies, to penetrate deeper into the human body.

The inventors of RASIR, have found that certain Wideband Chaos Generating Material (WCGM) comprising special substances, demonstrates the function to transform higher frequency RADAR signal into lower frequencies, able to reach inside, thru the human body, and in combination. The RADAR signal transformation may be a result of natural impedance and resonance circuits within the Wideband Chaos Generating Material (WCGM), or the impedance and resonance circuit may be a result of the structure of the material it-self or designed by purpose as made possible using metamaterial. Metamaterials and Wideband Chaos Generating Material (WCGM), resulting in physical signal wave interference, transforms the signals by combining frequencies into new frequency spectrum by signal wave interference resulting from at least two RADAR waves being combined, and where the waves are having slightly shifted in phase, frequency, or both.

The RASIR RADAR receiver and antenna is configured to at least receive RADAR signals at a wideband frequency spectrum reaching well into the centimeter RADAR bandwidth. A MIMO RADAR antenna for millimeter RADAR may have an antenna array matrix or line vectors, able to pick up half wave signals of wavelengths reaching 30 mm, thus making the receiver able to cover a wide bandwidth carrying wavelengths well above the millimeter RADAR frequencies. This makes it possible to receive above millimeter RADAR wavelength signals passing thru the human body.

Wideband Chaos Generating Material (WCGM) 30 demonstrating a signal transformation capability translating TIS into ATIS signal, may be implemented in the form of a designed metamaterial interference object (MMIO) 31. Some natural objects (NO) 32, may act as target objects having similar signal transformation functions from TIS to an Altered Transmitter Interrogation Signal (ATIS). Once discovered, the RASIR system interrogates the WCGM, MMIO and NO for distance, location, and for frequency response sampling model, describing the transformation from TIS to ATIS. As the sampling model is predicting the TIS to ATIS transformation, these WCGM, MMIO and NO can then be used for remote interrogation of other target objects. The WCGM, MMIO and NO are then functioning as slave TIS RADAR signal generators, emitting ATIS signals for target object interrogation.

The use of WCGM, MMIO and NO as slave signal generators, allows the RASIR to reach both shielded target objects (STO) 27, and target objects that cannot directly be reached and interrogated by the primary RASIR transmitted RADAR TIS. Here, the WCGM, MMIO and NO may relay the primary TIS 33 sent from RASIR's Tx Antenna 17, towards the WCGM, MMIO and NO, which then transforms the TIS into an ATIS, or just reflects the TIS 33 into a secondary ATIS signal 36 that interrogates the Target Object 22. The Target Object 22 may then transfer the signal to the RASIR receiving antenna 18 either via the WCGM 35 and then transformed as a signature reaching the RASIR receiving antenna 18, or directly to the RASIR receiving antenna 18, or in a combination.

In a MIMO millimeter RADAR, signals will follow multiple paths that may be separated in time or space, by RADAR sector and in a direction defined by a RADAR lobe shape. In a MIMO antenna arrangement, the transceiver antenna may use RADAR signal Precoding calculation to optimize its emitted Tx signal, see:

$$\begin{matrix}“ A practical precoding approach for radar / communications sharing \vert IEEE Conference Publication \vert IEEE Xplore ” \\ https : // ieeeexplore . ieee . org / abstract / document / 6636787\end{matrix}$$

Precoding takes place on the RASIR RADAR transmitter side. By using a digitally controlled array or matrix of transmitter antennas, and similar arrangement for the receiver side, to extract signals transmitted to, and received from, a specific target object, based on an angle of arrival in a MIMO antenna arrangement. The distinct frequency response sampling model of an IRS is used to find a signature sampling model alignment match with a signature sampling model stored in CCFRP as Reference Interrogation Signatures (RIRS), which further helps to be able to particularly recognize IRS response sampling models that are, distorted due to noise, irrelevant signal interference, doppler shift due to target object's velocity and imperfections in the material and RADAR ray tracing scenario.

Once a well-recognized IRS has been discovered to match a distinct reference RIRS in CCFRP, it is then possible to further improve the precision for the RADAR distance, angle of arrival, and time-of-flight TOF. When the RIRS predict an exact shape of the expected IRS, and even when no previous RIRS exits in CCFRP; RASIS may use the IRS or RIRS signal sampling model, to determine a better precision distance and angle of arrival matching when RASIR is equipped with a MIMO antenna arrangement.

With an improved identification and detection step, as mentioned above; RASIR may then further apply the method remove the recognized RIRS signal component, from the incoming IRS signal. This lets RASIR further reveal any sub-ordinate, previously depressed target object signature IRS component, and other types of target objects in the RADAR's field of view defined by its lobe shape and antenna arrangement.

The mentioned method in RASIR may thus increase the SNR for target objects, reduce or eliminate irrelevant signal interference, otherwise hidden behind target objects having an overwhelming strong RADAR reflection signal.

The method is for example useful when interrogating an WCGM for its influences from a nearby sensor type of target object in its surrounding. First the RASIR transmits a TIS, to map the WCGM's IRS as RIRS and then removes the IRS from the WCGM, then after consecutive TIS interrogations of the WCGM resulting in IRS, it is possible to detect remnants from a nearby TO interacting with the WCGM thru mutual resonance and impedance binding. This lets RASIR map locations of, recognize, and categorize other target objects, using a remotely located Wideband Chaos Generating Material (WCGM), MMIO, and NO. A natural object NO in the form of a metallic natural occurring object, may act as a Wideband Chaos Generating Material (WCGM) due to its shape. For example, a metallic object having sharp corners, or spike formed metallic parts, may be induced to make signals according to the object's natural electromagnetic resonance frequency, which then may generate both higher, and lower electromagnetic emission, and even sparks, transmitted from corners, spikes, and its structure.

In essence, RASIR makes opportunistic use of signal interference by mapping the environment for target objects and using these target objects including Wideband Chaos Generating materials (WCGM), as signal source for TIS, where even the remote WCGM signal source emitting a secondary TIS that is an Altered Transmitter Interrogation Signal (ATIS) 36 which can contribute with localization reference information by its geometric relative location to the target object. This extra ATIS information element may support the determining the location of a remote target object, using signal triangulation between the locations of the Tx Antenna, WCGM location, Rx Antenna as a mean to locate the target object location.

Mentioned RASIR function is challenging the view of earlier RADAR systems, including the patent application: WO2018206934A1, REAL-TIME LOCATION SENSING SYSTEM. The traditional view is that signal interference is as a problem leading to weak SNR, disturbances, and even fading RADAR signals due to phase shift during reflection. RASIR solves the need for a millimeter RADAR that can penetrate thru the human body, by using a wideband range of wavelengths, beyond that may stretch beyond the millimeter frequency band. RASIR makes use of Wideband Chaos Generating Materials (WCGM) to increase its RADAR bandwidth for its transmitter's, allowing its RADAR to cover a larger bandwidth than originally transmitted by the RADAR Tx antenna. This lets RASIR, perform interrogation of target objects for resonance frequencies, and frequency dependent attenuation, beyond the originally transmitted narrow frequency TIS signals, emitted by the RADAR Antenna. Hence by using Wideband Chaos Generating Material (WCGM), RASIR can interrogate its environment for target objects, using a wide frequency bandwidth. As a result of RASIR using proximate WCGM, RASIR can improve its ability to determine location, and position of target objects (22-29) being interrogated. Precision and resolution of the signal can be increased by the presence of WCGM, generating ATIS signals having a unique wideband signal signature.

The RASIR system makes use of sampling model matching of transmitted interrogation (TIS) signals and received interrogation signature (IRS) by comparing the signature's sampling model with pairs of sampling models handled by RASIR's CCFRP database function. Matching may be performed by applying least square error methods on each sample and for several samples at different frequencies. Other alternatives are cross correlation, auto correlation and different sampling model alignment estimation methods. It is known that unique concentrations of substances (sugar, glucose, starch, salt) and water solutions follows unique mathematical models. Examples are frequency response sampling models aligning with a linear function, for metallic material; a quadratic second degree polynomial equation function for salt and water solutions; and a third-degree polynomial equation function for sugar- and carbohydrate-solutions with water. Metamaterials may further be designed to demonstrate other distinct signal signature sampling models, improve efficiency in remote interrogation of target objects, using the metamaterial's generated Altered Transmitter Interrogation Signal (ATIS). Still RASIR may extract equation coefficients to match a certain equation function sampling model expressed as IRS and RIRS, for later use in patter matching of target object signatures with RIRS in CCFRP.

Yet another tool for estimating similarities and directly accessing information content in CCFRP is to combine RASIR's Pattern Matching Function (PMF) with CCFRP using neural network pattern matching techniques, and similar Artificial Intelligence inspired matching and searching functions, and hardware acceleration for AI applications and neural network execution and training of new sampling models to be matched in the future. In a configuration where RASIR uses a neural network, both the PMF and CCFRP can be integrated in the neural network function, such as a Convolutional Neural Network (CNN) and Deep-Learning Neural Network (DLNN), as well as other artificial intelligence based learning algorithms; to further learn and register new target object categories and to generate new RTIS and RIRS pairs for registration in CCFRP, when RASIR is set in a learning mode 60. During recognition and localization of target objects, RASIR may then be set to a learning mode on, or off, depending if RASIR is to learn new target object, and WCGM objects on the fly; or only to recognize and localize previously taught target objects 22-29 and WCGM objects 30-32.

Furthermore, the RASIR system may interface with applications making use of target object data such as target object position, target object material base classification, state, velocity, rotation, physical state, moisture, water concentration, mechanical state, time elapsed since tag was activated, flow of fluid and existence of fluid in body, remote sensing of physical properties, remote sensing of electromagnetic material properties and electrical states.

Systems and applications benefiting from integration with RASIR are many such as:
a) Tracking of Target Objects in the form of special tags having a context unique identity, based on a its non-linear frequency dependent response signal sampling model IRS, and signature sampling model matching with existing sampling models from earlier recorded target object's (TO) stored as at least a RTIS and RIRS pair defining a response signal sampling model, with optionally more characteristics, in CCFRP 12.
b) Ego motion calculation and position tracking using RADAR-based Simultaneous Localization And Mapping (SLAM) of own position, movement- and rotation-vectors.
c) Recognition of electromagnetic Target Objects material based on frequency response characteristics, which is matching TIS and IRS in CCFRP. This lets RASIR recognize salt or sugar solutions.
d) Categorization of electromagnetic Target Objects material based on frequency response characteristics, which is matching TIS and IRS in CCFRP. This lets RASIR detect and categorize sugar solutions and to estimate related Brix-value for the sugar solution.
e) Calibration of RADAR and object position determination using a measurement probe stick 700, also for definition of areas and for information guidance for autonomous mobile robots.
f) Moisture detection in High Frequency Detection (HFD) Diapers, and monitoring system, alarm system, and diaper logistics tracking and markings; also using Wideband Chaos Generating Material (WCGM) for extended signal reach.
g) Medical care inspection system for tracking of internal body functions, using Wideband Chaos Generating Material to generate RADAR interrogation signals that will reach thru a human or animal body; accompanied with presentation and warning systems for analysis and tracking of water content in organs, blood system, blood cloths and blood fat; congestions in intestines and blood circulation system; and heart beats.
h) Identity recognition of geometrical tags and logistics, where recognizable TOwNLF based material can be used to signal a code value, similar to how a barcode, or QRC would signal a digital identity value within a certain context.
i) Automatic recognition and classification of all type of target objects detected, when the PMF and CCFRP are set in a learning mode, as a system to automatically create an image of a scene with unknown target objects. This makes RASIR function as a 3D camera that also recognizes target object characteristics, in multiple dimensions including direction of travel and rotation if any, as well as the target object's frequency response, a RADAR electromagnetic frequency spectrum reflected from the target objects, including classification of last known location.

The RASIR system presented, overcomes the problem of performing target object (TO) substance classification and identification and position measurements using millimeter Frequency Modulated Continuous Wave (FMCW) RADAR in an environment with signal interferences, without having to depend on the target object's geometrical shape and aspect angle of the target object. But RASIR is not limited to FMCW principles, and may mix different techniques to interrogate target objects, such as time of flight (TOF) RADAR, narrow frequency scanning RADAR transmitting bursts of essential narrow frequency sub-band for interrogation, frequency by frequency. Yet another technique is to use Orthogonal Frequency Division Multiplexing (OFDM), which is not feasible for frequency based interrogation of target objects, but useful for elimination of signal interferences, see:

“Comparison of Automotive FMCW and OFDM Radar Under
Interference”
https://research.chalmers.se/publication/522488/file/522488_Fulltext.pdf

RASIR may also use a Frequency Hopping Signal (FHS) as a method to generate useful Transmitter Interrogation Signals. Also, any waveform defined as a RADAR Chirp, may also be used to interrogate target objects using different TIS to sample related IRS for further matching in PMF and CCFRP.

The WCGM can transform a received TIS into a reflected and transformed Altered Transmitter Interrogation Signal (ATIS) having a minor frequency shift. As the first TIS and the WCGM generated ATIS signal waves are combined into a combined second TIS comprising the first TIS and the ATIS, reaching a target object, the combined second TIS may hold new frequency component due to wave interference effects resulting from the combination of the first TIS and the WCGM generated ATIS.

WCGM can generate signal interference according to mentioned principles; as a signal translation function also shifting frequency band; as a resonance circuit producing a time delayed frequency response reverberation of a generated signal.

In the case the WCGM Material is a metamaterial, then the metamaterial's surface may be configured as a reoccurring mesh of resonance circuits, antennas, or with a sampling model resulting in real signal interference and unique signal reflections and refractions. Depending upon the RASIR application and system use, different WCGM, may fit the system need. One example is the need to generate signals having longer wavelengths for tissue and textile penetrating RADAR signals. Yet another usage of WCGM is to flatten out narrow TIS interrogation frequencies, to generate a broader spectrum near the target object, to increase the bandwidth of the TIS, even beyond the millimeter RADAR bandwidth and spectrum.

Contrary to traditional RADAR systems which performances are degraded by signal interference, the RASIR system uses interference signals, signal reflections and frequency band scatter from interference emitting objects near the target object, as a signal source for improvements in object recognition, and as a mean to shift frequency band near a target object to enable frequency signature sampling model recognition of target objects beyond the original transmitted RADAR frequency bandwidth. Interference objects may be placed near a target object to be investigated, or exist in the natural environment, as a discoverable resource. The RASIR system may be used for remote target object sensing using millimeter wavelengths without relying on target object dimensions, as not limited to but typically non-linear frequency sub-band reflection sampling models are analyzed to identify the frequency response beyond the transmitted Tx signal, to match known characteristic frequency dependent signal characteristics.

Wideband Chaos Generating Materials (WCGM) are not limited to antenna structures such as metamaterials, as natural objects may also share similar behavior, and other occurring objects may pick up frequencies and scatter interference frequencies, typically around a corner of a metallic conductive structure on a nonconductive substrate.

Metamaterials for RADAR applications like RASIR exists in many configurations, but may for example consists of a conductive foil comprising small conductive c surrounded by larger mirrored 3 (mirrored c-shape), where the small c's inner radius is about 2.0 mm, and the thickness of the small c is about 1.0 mm, and the space between the inner and outer c is about 0.1 mm, where this pattern is re-occurring at a distance of about 10.0 mm in a horizontal and vertical grid pattern. The small c and large c's may have a narrow opening in in its otherwise ring-shaped form, thus creating a surrounding almost one turn coil with a gap acting as a capacitor, with an ability to induct current and by high frequency magnetic flux changes between the two c and C circuits, thus creating an interference signal at a new frequency. The material may also replicate and transform signals from the new frequency spectrum back to the original frequency spectrum for detection by the RASIR RADAR receiver.

The mentioned metamaterial structure (MMIO) 31 is just one example of a designed metamaterial to be used as a Wideband Chaos Generating Material (WCGM) 30 in the RASIR RADAR 2 system. As mentioned, natural occurring objects (NO) 32 may also be used as Wideband Chaos Generating Material (WCGM), by placing the natural object (NO) near the target object on purpose. Sometimes a natural objects (NO) occasionally not placed at a location by purpose, may be discovered by the RASIR system by first detecting the presence of the natural objects (NO) and then by analyzing and recognizing, actually remembering the non-frequency dependent signal characteristic of the NO. RASIR may then make use of the natural object (NO) behaving as a Wideband Chaos Generating Material (WCGM) as a mean to interrogate proximate target object for its frequency dependent signal response. Hence the “natural objects” (NO) 32 behaves as Chaos Generating Material (WCGM), that generates an enhanced wider frequency spectrum signal, to improve localization precision and precision in identification and recognition of the target object (TO) 22, and other similar RASIR target objects 23-32.

Different material compositions can demonstrate unique frequency dependent signal response sampling models in both target objects, WCGM, as well as target objects in the form of unique RASIR RADAR tags carrying context unique tag identities.

The frequency dependent signal response IRS and RIRS normally demonstrates a frequency response signature sampling model in the frequency domain with a magnitude that are:
a) linear frequency dependent as demonstrated by metallic objects and surfaces;
b) non-linear frequency dependent demonstrating a power of ×2, a square polynomial function form, as for common for salt and salt solutions;
c) non-linear frequency dependent demonstrating a power of ×3, a third degree polynomial equation form, as common for sugar, glycose and such solutions with varying concentrations;
d) a result of combined signal signature sampling models, resulting from a heterogeneous target object material composed of different materials substances which in combination are demonstrating a unique signal signature sampling model. Such heterogenous target object model material can be used in RASIR tags to communicate a unique tag identity;
e) combined tags with exposed geometrical sections having different uniquely recognizable frequency response sampling models, which is a geometrical combination of the above material characteristics. For example, a matrix or any other geometrical configuration, of target object tags can communicate a certain code value, depending on the coding theory chosen. Multiple tags can be combined, having a known geometric displacement, where also individual distances between tags can communicate a code value, such as an identity of a package code, a serial number, or article number. Such coding of for example a box, product, or article, may be integrated with the product as such. For example, a product such as a chair could be coded for recognition of article, while also communicating its orientation of the product, chair, in relation to the RASIR system's Tx and Rx antenna configuration.

RASIR is using a TIS Generator TISG to generate different TIS signals, which in turn defines how the RADAR subsystem's Waveform Generator (WG) is to operate. As mentioned, TISG may generate alternative TIS datagrams that results in a better differentiated IRS, based on matching results in PMF. For example, if PMF finds multiple candidates of RTIS and RIRS pairs in CCFRP, but cannot differentiate these clearly, then the RASIR Control System (RASIR CS) may use CCFRP and PMF matching results to determine which new TIS to generate in TISG for a best differentiating interrogating result IRS, for the situation at hand. This can be seen as an outer improvement loop selecting best possible TIS for interrogation.

The RASIR system's inner control loop would typically transmit TIS as a Tx signal 256 times and then receive 256 IRS to be co-sampled to establish a TIS IRS pair for further analysis in PMF and CCFRP. TISG may also decide on type of interrogating waveform for TIS, such as usage of narrow frequencies, signal sub-band bursts, and several types of Chirp signals, including exponential shaped Chirp signals sweeping over frequencies over time according to an exponential function to express a rapid increase in frequencies, to generate efficient TIS interrogation signals.

TISG may further generate TIS signals that are best adapted for localization of TO distance and angle of arrival, using Time of Flight (TOF), FMCW, or more complex TIS signals as mentioned above. TISG also controls the bandwidth to use when translating the datagram TIS to Tx signal representing the TIS, as generated by the RADAR Waveform Generator.

Once a TO is recognized by the PMF and CCFRP, the information can be compiled into a RASIR Target object Localization and Characteristic (RASIR TLC) datagram, that is forwarded to a RASIR Interface (RASIR IF) for consumption and interaction in a RADAR target object presentation system, a TO recognition system, a remote target object acting as a TO sensor function to measure physical or chemical parameters, including moisture that is the percentage of water to salt and sugar in a solution, time since activation of tag as a function of moisture evaporation, mechanical shielding of tag, bending of metamaterial structure of tag resulting in a change in frequency response IRS.

According to one embodiment, the Wideband Chaos Generating Material (WCGM) comprises a designed metamaterial. In another embodiment, the WCGM comprises a natural occurring wideband chaos generating materials, here denoted Natural Objects (NO) 32 acting as a metamaterial. An example of a NO, can be found as a sugar-water solution where an effect of the metamaterial is a signal transformation and reflection to a new frequency spectrum, an absorption of different frequencies according to a non-linear frequency dependent sampling model, or an impedance effect created as a result from a dipole resonance effect in a water-substance solution such as a sugar-water solution, or within the natural material structure, or as a compound of target object materials.

In one embodiment of the RASIR tag, based on a Target Object with a Non-Linear Frequency dependent signal response sampling model (TOwNLF), the RASIR tag 1001 comprises a TOwNLF properties such as a sugar-water solution 1003 in a sealed envelope 1002, optionally carried by a carrier and reflector surface 1004. Such designed RASIR tag is feasible for designing Target Object tracking systems 1005, where tags 1001 can be tracked by a SISO, or preferably by a MIMO RASIR RADAR system.

In one embodiment, the sugar-water based mm RADAR tag 1001 has a non-linear frequency dependent mm RADAR reflection, attenuation, dipole resonance, resulting in a non-linear frequency dependent response signal from a narrow mm RADAR sub-band.

In one embodiment, the composition of a RASIR tag's 1001 TOwNLF substance 1003 may be composed of WCGM with TOwNLF characteristics.

In one embodiment target objects (TO) and RASIR tags are demonstrating a linear frequency dependent RADAR reflections signature.

In one embodiment a RASIR tag 1001 is comprising a non-metallic material and sugar-water based solution 1003 with a known Brix value registered in CCFRP together with matching RTIS and RIRS characteristics. This lets the RASIR system's PMF with CCFRP recognize the RASIR tag's unique identification and its data record in CCFRP.

In one embodiment a RASIR tag Target Object may comprise non-metallic material and sugar-water based solution with a Brix value that may be affected by presence of a water-based fluid such as a body fluid or urine.

In one embodiment a RASIR tag Target Object such as a HFD Diaper 801, comprises a non-metallic water absorbing material, and where in its IRS is recognizable and predictably changed under presence of a certain percentage of a water-based fluid such as a body fluid or urine. This lets the RASIR system recognize the amount of water percentage from a distance from such a water sensitive tag or HFD Diaper 801.

In another embodiment, the HFD Diaper 801 changes its a nonlinear frequency dependent IRS response signature sampling model at different water concentration, while being recognizable by RASIR's PMF and CCFRP.

In one embodiment, the RASIR system can locate the distance to, and angle to, a reflecting target object 22 of any kind of target object 23-32, or a RASIR tag 1001, in relation to the RADAR Tx and Rx antennas.

I another embodiment, the RASIR system makes use of a method where the TIS and IRS, as well as RTIS and RIRS pair is used for sampling model matching in the time domain, or frequency domain to identify a relative distance and location of the target object in relation to the Rx and Rx antennas.

In one embodiment, the RASIR method for recognition of a Target Object with Non-Linear Frequency (TOwNLF) dependent response signature, comprises the steps:
A) Transmission of a narrow sub-band frequency mm RADAR signal, a narrow band Chirp signal TIS;
B) With the emitted signal, hitting a target object TO, where in the target object returns a frequency transformed narrow band Chirp signal (IRS) having a frequency set and signal strength profile different from the at the target object received narrow band Chirp signal;
C) At the mm RADAR receiving the from the target object reflected signal Rx 34;
D) Mixing 20 said narrow band transmitted Chirp ITS signal and received target object reflected signature IRS;
E) Optionally, filtering the mixed signal by sub-band, similar to using a band pass filter, into a filtered mixed signal, although frequency filtering in a frequency domain is made very easily;
F) Transforming any mixed raw signal data, such as RADAR pulses remaining from time domain, such as time-of-flight pulse data, to a frequency domain, using a Fast Fourier Transform (FFT) 8 or similar function, and mix with other frequency domain signal data into an IRS 9 signal datagram. A transformation of RADAR signals from a time domain to frequency domain would take place early in a typical mm Wave RADAR, hence the FFT step can be omitted in most implementations of RASIR;
G) Identify the only beat frequency (one position) for the target object, by obtaining chirp responses from the target object (and via any WCGM if precent as a signal relaying meta material), by scanning in different sub-bands in conjunction of FMCW method;
H) Matching the FFT IRS at the transmitted narrow frequency band defined by the TIS, using a sampling model matching method (PMF) 10 based on one of the methods:
   h1) Convolutional Neural Network (CNN) based matching
   h2) Particle filter
   h3) Least square alignment method
   h4) Singular value decomposition (SVD);
I) Matching the TIS and IRS with CCFRP 12 Database records holding signal reference sampling models as pairs of at least RTIS and RIRS;
J) Optionally if previous RTIS RIRS pairs could not be found in CCFRP, then looking up and storing newly found characteristics as a new record in the CCFRP 12 database function;
K) Classifying each found spectral and mm RADAR response signature identified in CCFRP database based on signal signature sampling model matching related to actual TIS, IRS and RTIS, RIRS pairs;
   and optionally using an analytical signal analysis to determine a substance composition, such as a Brix-degree for a value in a sugar-water solution composition;
L) Integrating new TIS and IRS signal information data and signal response data found in relation to the actual RASIR tag's signal signature response model, as a data record transferred to the RASIR TLC as datagram, providing access to previous signal analysis data, correlated with the same RASIR tag identified based on recognized location, signal characteristics, or both;
M) Re-iterate using a new adapted TIS signal comprising a Chirp signal, a new sub-band frequency, that either repeats previous TIS, or differs from the previous TIS; and
N) returning to step A) above.

In one embodiment, the RASIR system calculates a TO tag identification value based on TO IRS, or by evaluating multiple IRS corresponding to a geometrical configuration and constellation holding multiple TO tags, wherein the identification value is determined either based on the IRS, IRS found in CCFRP, or in the event of a multiple tag configuration, based on multiple TO elements individual IRS values, or by making use of geometrical distances for calculation of a tag identification value.

In one embodiment the RASIR system can recognize target object signal response data IRS under radiation from an earlier detected, localized, and analyzed Wideband Chaos Generating Material (WCGM), to further enhance signal response characteristics of the target object.

In one embodiment, RASIR uses a method for non-invasive body data measurement, sensing, tracking, monitoring, or a combination, by transforming TIS Tx signals to a new frequency with wider range of wave lengths using a metamaterial (MMIO), a Wideband Chaos Generating Material (WCGM) or a natural object (NO) with similar frequency transformation functionality.

In one embodiment, the RASIR system and method can measure and detect a TO location, distance, RADAR signature IRS, RADAR interference signature of a TO such as nonlinear frequency dependent attenuation and resonance, and combinations of these, where a water-sugar solution of a specific sugar-water concentration, that is a certain ° Bx (Brix) value and IRS demonstrating a non-linear frequency dependent attenuation signature sampling model.

In one embodiment of the RASIR system, a TO having a specified Brix-value, can be recognized based on its characteristics IRS sampling model using signal processing and matching.

In one embodiment, the PMF makes use of a Convolutional Neural Network (CNN), optionally with a Deep Learning Neural Network function to enable self-learning of methods for complex sampling model recognition of known or re-occurring target objects.

In one embodiment, where in the RASIR system, contrary to traditional RADAR systems where a high signal-to-noise ratio (SNR) and reduction or elimination of non-relevant background interference are needed to efficiently discriminate actual targets from noise signals; the RASIR system instead uses interference signals from an Wideband Chaos Generating Material (WCGM), as an extra information and signal resource, that when analyzed contributes to an improved understanding of the geometrical environment and the target object's electromagnetic signal characteristics being monitored by RASIRS millimeter RADAR, in relation to RASIR's Tx and Rx antenna configuration.

In one embodiment the RASIR system makes an analysis of an ITS specified Chirp signal reflected from a target object TO, and where the Chirp signal is typically a frequency modulated continuous wave (FMCW) signal sweeping from one frequency to another at the about same amplitude.

In one embodiment the RASIR system makes an analysis of an ITS specified Chirp signal reflected from a target object TO, and where the Chirp signal is typically an amplitude modulated continuous wave (AMCW) signal sweeping from one frequency to another at the about similar amplitude.

In one embodiment the RASIR system recognize characteristic target objects with non-linear frequency (TOwNLF) dependent response signature, where in the frequency dependent TO signal attenuation signal is sampled as an Interrogation Response Signature (IRS) where the sample defines a non-linear frequency dependent sampling model in a frequency domain presenting a magnitude and a phase for each frequency.

In one embodiment, the RASIR system can identify TO:s having non-linear frequency dependent RADAR signatures. TO:s having non-linear frequency depended RADAR signature may be based on a water solution but may be any electromagnetic material that can act as a dipole. Such materials which have non-linear response should be considered when selecting TO:s such as biomaterial, organic material, and even designed metamaterial-based target objects.

Target Objects (TO) being based on a result of a composition of water solutions with a determined composition of percentages of substances of sugar, glucose, starch, salt, or a combination. This may result in a TO with a unique characteristic non-linear frequency dependent RADAR signature response sampling model. Such a TO can be interrogated using a TIS, for which a received RADAR chirp would finally result in an IRS. Thee received chirp signal, may be sampled as a signal, and transformed from a time domain into a frequency domain, for example using a Fast-Fourier Transform implementation or similar, and then captured as an IRS.

Target Objects (TO) having a unique characteristic non-linear frequency dependent RADAR signature response sampling model can be designed using metamaterial, and using other compositions, and even result in apparently solid form.

The representation of the IRS in the frequency domain will present magnitudes in a frequency plane, for an at the RADAR transmitter controlled sub-band frequency signal TIS, resulting in a received TO signal IRS with a characteristic frequency sampling model having new frequencies due to one of or a combination of:
a) hydrogen dipole interference with the RADAR signal, for example as a result of a determined amount of water in a sugar-water solution having a certain Brix (° Bx) value;
b) frequency dependent attenuation of different material being penetrated by the RADAR signal;
c) Wideband Chaos Generating Material (WCGM) reflecting and distorting the RADAR signal thus altering the frequency and phase.

In one embodiment the metamaterial (MMIO) interference object transforms and increase the band frequency, interrogating nearby target objects (TO).

In one embodiment, the WCGM is designed and configured to translate a first set of frequencies using a translating antenna comprising a RADAR receiving first antenna translating an electromagnetic radio wave at a first frequency band into an electric current which then drives a second antenna resonating at a different frequency.

In one embodiment, the WCGM in the form of an MMIO, has a second antenna that transmits the signal as an electromagnetic wave at a second frequency band which may later hit a TO and reflect to the second antenna now acting as receiving antenna, were in said frequency translating antenna's first antenna then transmits the signal at the first frequency band to the RADAR receiver.

In one embodiment, RASIR uses an Wideband Chaos Generating Material (WCGM) as a remote slave antenna, by first transferring a primary TIS defined RADAR signal at a first frequency band, and then letting the designed WCGM, replicate the first signal at a first frequency band translated into a second frequency band, to make it possible to at a distance measure RADAR signal responses from a TO material over more frequency bands, thus enabling collection of more information about a RADAR signature IRS emitted from a TO material being investigated.

In one embodiment the RASIR in MIMO configuration controls the time occurrence of the first signal Chirp, or sub-band frequency signal burst being transmitted, and then identifies and screens and dispatches relevant TO signals for a known location, thus improving and incrementally finding a better information source which can be dispatched for analysis of IRS from each TO signal, pre-separated by the RADAR function's ability to detect Time of Flight (TOF) and angle of arrival.

In one embodiment the WCGM may be composed of a natural object (NO) having a material exercising RADAR signature properties similar to other Wideband Chaos Generating Materials (WCGM), where in its intrinsic antenna materials results in a translation of a RADAR frequency signal from one sub-band frequency into a new sub-band frequency due to its intrinsic antenna configurations, such as materials and objects of conductive material having a shape that acts as receiving antennas with imperfections thus resulting in burst of new interference signals, resembling sparks at the edge of antenna corners. Similar effects may be found in other objects.

### BRIEF DESCRIPTION OF DRAWINGS

The invention is described, by way of example, with reference to the accompanying drawings, which follows.

FIG. 1 gives a presentation of a new RADAR system able to detect and analyze target objects based on their frequency response sampling model when interrogated with different RADAR signals; and target objects, all demonstrating useful functions for object detection, recognition and localization mapping including Wideband Chaos Generating Material (WCGM) and metamaterial having useful functions to improve precision and system capabilities.

FIG. 2a shows a prior art RADAR system where interference signals are problematic and reduces Signal to Noise Ratio (SNR) for the RADAR system.

FIG. 2b shows a new proposed RADAR Signal Interference Recognition (RASIR) system interference signals as resource, to increase the bandwidth for target object frequency-based interrogation, and as slave signal sources for improved mapping of target object locations.

FIG. 3a shows a target object in the form of a non-linear frequency dependent RADAR signature generating target object being a RADAR tag, a Target Object with Non-Linear Frequency (TOwNLF) dependent RADAR signature frequency function, and signal signature sampling model.

FIG. 3b shows a positioning measuring probe for integration with a RADAR system capable of tracking target object based on frequency response signature target object tags.

FIG. 4a shows a High Frequency Detection (HFD) diaper moisture alarm and monitoring system measuring moisture sensitive target object tags as at distance, based on a frequency response signature IRS that changes in relation to moisture and other substances in the diaper. The diaper may as well be replaced by a wound dressing serving a similar purpose.

FIG. 4b shows a second diaper moisture alarm and monitoring system configuration measuring moisture sensitive target object tags as at distance, based on a frequency response signature IRS that changes in relation to moisture and other substances in the diaper. In this second diaper monitoring system configuration, the RADAR control and analysis system (RASIR) makes use of a Wideband Chaos Generating Material (WCGM) 819 to increase RADAR bandwidth and ability to penetrate material at wider range av wavelengths, to improve precision and ability to reach thru material when interrogating the diaper material and its moisture binding material, such as sodium polyacrylate (SPS), known as water gel powder, and similar substances like polyacrylamide.

FIG. 5 shows a millimeter RADAR system for non-invasive inspection of a human or animal body using Wideband Chaos Generating Material (WCGM), such as metamaterial to enhance frequency spectrum to reach into and thru a body to typically measure water concentration in organs, intestines, lungs, and blood vessels.

FIG. 6a-c shows sampling model matching of transmitter interrogation signal (TIS) and interrogation receiver signature (IRS) in multiple dimensions, where pairs of transmitted and received signals are analyzed and presented as a sampling model, and then matched towards previously stored reference transmitter interrogation signal (RTIS) and reference interrogation receiver signature (RIRS), stored in the Catalogue of Characteristic Frequency Response Patterns (CCFRP) database function, or equivalent mechanism.

FIG. 6a shows a transmitted TIS 200 and received IRS 201 in a matching window 202, where the signals have been transformed thru an FFT and presented in a frequency domain, which is a frequency signal magnitude coordinate system, for further sampling model matching of signal signature sampling model, independent of the distance between target object and RADAR antennas.

FIG. 6b shows two matching windows 202 and 212 after having interrogated the target object using two different TIS.

FIG. 6c shows a in matching window 202, the matching of the pairs TIS and IRS with response stored in CCFRP as RTIS and RIRS sampling models for different target object candidates having different frequency response characteristics given a specific TIS.

FIG. 7 shows a Method for basic TO recognition.

FIG. 8 shows a Method for recognition of TO using WCGM.

FIG. 9 shows a Method for recognition of STO using WCGM.

FIG. 10 shows a Learning function for mapping and categorization of new target objects and tags based on frequency response IRS detected in a scene visible for the RADAR.

FIG. 11a-g shows different RADAR scenarios, where the RASIR RADR is equipped with a Single Input Single Output (SISO) or Multiple Input Multiple Output (MIMO) antenna configuration; and Target Objects TO with and without non-linear frequency dependent response signature models, Wideband Chaos Generating Material (WCGM), and Wideband Chaos Generating Material as intermediate signal generator of ATIS 36, and transceiver for RADAR signals reflected by Target Objects (TO) 35.

### DESCRIPTION OF EMBODIMENTS

In the following section, a detailed description of the RASIR system with methods, TIS/IRS sampling model matching methods for RTIS, RIRS pairs in CCFRP, target objects, Wideband Chaos Generating Materials (WCGM), RADAR tags and application for different usage scenarios, are provided.

### The RASIR System

The present invention, the RASIR system with methods and target objects, relates to a millimeter Radio Detection and Ranging (RADAR) Signature Interrogation and Recognition system, hence the acronym RASIR. RASIR includes methods for recognition and classification of target objects, especially for target objects demonstrating a characteristic non-linear frequency response signature sampling model, during signal interrogation with a specific transmitter interrogation signal (TIS).

The RASIR system is configured to recognize target objects using, signal signature sampling model matching, where received RADAR signals are compared and matched with earlier recognized RADAR response signature sampling models, stored as database records in a database function called, the Catalogue of Characteristic Frequency Response Patterns (CCFRP).

RASIR can make use of signal interferences emitted from Wideband Chaos Generating Material (WCGM) to improve precision, categorization, and recognition of target objects. Wideband Chaos Generating Material (WCGM) objects can typically be based on a metamaterial structure, configured to reflect, and transform signal frequencies in the millimeter frequency band, into signals having an extended bandwidth preferably containing signal components with longer wavelengths, and back.

Interrogation signals of longer wavelengths, as generated by WCGM, lets RASIR reach and interrogate target objects otherwise blocked by millimeter RADAR wavelengths.

As the WCGM re-generated interrogation signal acts as an at least partially controlled extra signal source, this WCGM interrogation signals can be localized to have a certain position, from the WCGM generated altered transmitter interrogation signal (ATIS) are emitted towards a second target object. Hence, the RASIR system can interrogate remote, hard-to-reach, and shielded target objects (STO) 27 using an WCGM as slave interrogation signal generator ATIS, to perform triangulation of target objects. As the WCGM can also contribute to an extended bandwidth of ATIS in relation to the original TIS, this makes it possible to reach object that are otherwise not reachable on a millimeter RADAR frequency band.

RASIS is making use of a transmitter signal (TIS) based analysis of frequency response signature sampling model (IRS), and a Pattern Matching Function (PMF) that identifies similar transmitter-receiver (RTIS and RIRS) signals sampling models stored in the Catalogue of Characteristic Frequency Response Patterns (CCFRP).

The PMF is matching of sampling models based on target objects' response signature in the frequency plane, that is in the frequency domain, but can also perform matching based on signals in the time domain, also based on signal intensity, phase shift, doppler shift, or polarization sampling model, preferably using combination of matching aspects, fit for the type of target object being search for.

For example, if RASIR only searches for oranges, then it would typically use a sampling model matching method that is optimized for an orange's frequency response. If in another example a reflective surface is being studied, then it would be natural to study polarization of reflected signals, as reflections usually changes the signal polarization during reflection. Each unique type of target object may require its own optimization for best interrogation method and signal signature sampling model matching.

Designed RADAR signal reflecting target objects for the RASIR system, are denoted RASIR target objects (TO). The system makes use of RASIR Wideband Chaos Generating Material (WCGM), typically based on a metamaterial structure configured to reflect and transform signal frequencies of RADAR signals in the millimeter band over an extended bandwidth, and back.

The RADAR Signature Interrogation and Recognition (RASIR) is a RADAR control and analysis system, which can be configured, by selecting feasible hardware and software, to transmit millimeter RADAR signals with a wavelength from 0.1 to 30 mm, while receiving, depending on hardware and software configuration, and analyzing target object reflected RADAR signals of a broad frequency spectrum, typically having a wavelength from 0.2 mm to 150 mm (ranging into the 2.4 GHz frequency band). Preferably frequency bands for the RASIR system are: 3-7, 6-10, 8-12, 22-26, 50-54, and 60-77 GHz.

In traditional RADAR applications, noise, and interference signals from a Wideband Chaos Generating Material (WCGM) 30 and objects in the proximity of target objects, are seen as problematic signal disturbance, noise, and interferences reducing the signal to noise ratio (SNR), received from target objects being detected, range estimated, and sensed. The RASIR system and methods, instead turns such interference signals generated from reflections in Wideband Chaos Generating Materials (WCGM) and objects, into a resource for improved mapping, frequency response recognition, position triangulation, and to achieve an increased RADAR frequency bandwidth.

RASIR uses metamaterial, and other Wideband Chaos Generating Materials to translate millimeter RADAR signals to wider range of wavelengths to allow invasive measurement in the human body, and to reach thru materials otherwise not reachable by millimeter RADAR wavelengths.

This process makes it possible to measure moisture, as a function of RASIR measuring a solution-based material's components, which is a quantitative and simultaneously measurement of occurrence of different components. RASIR can measure substances inside a HFD diaper having pocket or volume with a fluid absorption material, for urine, pus, blood, glucose in the blood, water, and other body fluids. This lets RASIR support detection and remote measurement of urine, water, or other solution-based materials such as blood, and other solution concentrations, remotely using the RASIR system. Water based materials and solution-based materials are different materials. For example, blood is a solution-based material and not water-based material. RASIR can detect the components in the solution-based material, which is for example glucose in blood.

Meanwhile a traditional moisture sensor measures the moisture quantitatively; RASIR measure the solution-based material's components. This means that RASIR finds quantitatively and simultaneously different components existence. Accumulation of the amount of these components can be seen as quantitative substance and fluid concentration, thus also an indicator of a moisture measurement inside HFD Diaper 28.

The absorption material changes its non-linear frequency response signature sampling model according to different concentrations of urine, and water. RASIR's PMF can identify a predefined water concentration by finding a matching signature sampling model in CCFRP, or by performing interpolation and matching towards known signature sampling models in CCFRP to estimate a water, or sugar concentration, alternatively PMF may use a mathematical expression to translate the signature sampling model into a corresponding concentration level. Finally, PMF can make use of a convolutional neural network to perform sampling model matching, with the option to return a measured concentration value.

RASIR may also use Wideband Chaos Generating Material (WCGM), if available, to locally transform RADAR signals to longer RADAR signal wavelengths, able to reach inside a human or animal body. These lower frequency RADAR signals, which are here referred to as Altered Transmitter Interrogation Signal (ATIS), may then be used for measuring RADAR signatures from tissue volumes inside the body. By this method, RASIR can supervise and measure fluids in a wound dressing, body fluids, glucose levels, and fluid movements inside or near a human or animal body. Applications range from measuring the amount of urine in a urine bladder; water in the stomach, feces in the intestines; blood in blood vessels; to monitor movements in clogged blood vessels; and to monitor glucose level for diabetic care receivers.

The RASIR system may also act as a traditional RADAR with improved abilities to identify objects based on a signal signature sampling model, for tracking of objects in for example cargo tracking applications, where each object may carry an information element. In a RASIR system configuration for a logistics cargo tracker application, one may fixate an Wideband Chaos Generating Material (WCGM) on the outside of a corrugated cardboard box, or cargo container, to act as a mean to send TIS RADAR signals and reach inside the box or container, as an Altered Transmitter Interrogation Signal (ATIS), to further interrogate other target objects placed inside the box, and then to relay any signal back to the RASIR Rx receiver, for analysis of the IRS signal received.

The RASIR system may also be used as an improved system to resolve ego-motion calculations and to perform Simultaneously Localization and Mapping (SLAM) for robotic applications, detection of state changes to a certain tracked object, tracking of objects having multiple tags storing complex information, and in combination with objects with known dimensions, for more advanced information retrieval tasks. An improvement over prior art is that the RASIR system may make use of non-linear frequency response signals recognized by sampling model matching methods, and Convolutional Neural Network based matching of frequency dependent signals, to detect target objects independent of aspect angle and target object silhouette presented for the RADAR antennas.

### First Embodiment—RASIR Super System Integrating RADAR Functions and Target Objects

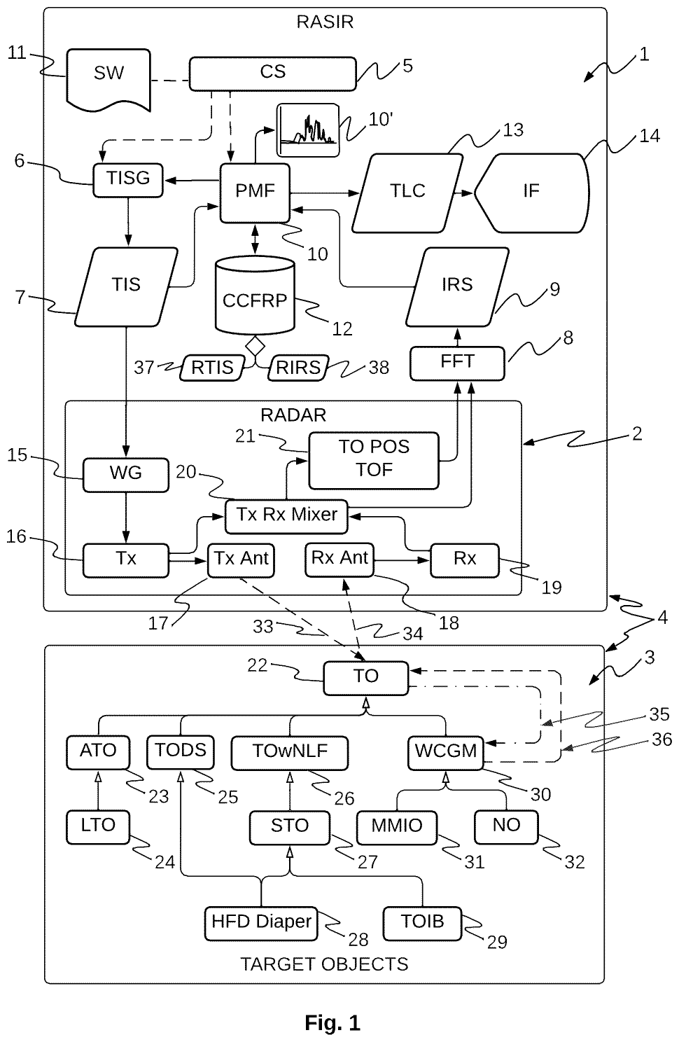

FIG. 1. Shows a first embodiment, the complete RASIR super system 4, integrated with its different types of Target Objects included in the Target Object and Model of Environment. As such the two parts form a RASIR super-system 4, combining a RASIR including its integrated RADAR system for target object interrogation 1, and the RASIR super system's target objects 3, which forms a lock and key concept.

The RASIR super system 4 is a millimeter RADAR system for target object tracking, localization, and categorization of target objects, in the most general definition of target objects as objects that may be interrogated by the RASIS super system, its RASIR subsystem 1, where target objects may be any type of object that results in a reflection back to a millimeter RADAR system. To further ease the classification of target objects, the classes of target objects 22-32 have been structured according to their function and characteristics RADAR signatures, principles for RADAR reflection, and how a RADAR signal reflects from the target object, generating a RADAR reflection signature, and a sampling model, which defines how a target object is recognized by the RADAR. RASIR does not only scan and analyze target objects based on RADAR Cross Section (RCS), but also makes use of a frequency response pattern generated based on a specific response pattern, called a target interrogation signal (TIS). Although the TIS is a datagram, that defines how a RADAR signal should be generated in the RADAR waveform generator function, the term TIS is also being used to describe the waveform being transmitted from the transceiver antenna, as the waveform communicates the TIS information as a message for interrogation of the target object in reach for the antenna. Also, the term Interrogation Receiver Signature (IRS), which is also a datagram, is also being used to express a signal being reflected from a target object.

As RASIR does not only use RCS for target object identification which is a sensitive method as a change in aspect angel for the target object's profile towards the RADAR antenna arrangement, with the drawback that a new aspect angle would result in a new RCS signature.

As a remedy, RASIR rather relies on each target object's context unique frequency dependent RADAR signature samplings model, and deviations from linear dependences between frequency and magnitude of RADAR energy reflected for each frequency. A special type of target objects (TO), called target objects with non-frequency linear response signatures (TOwNLF) demonstrates a useful non-linear model that may follow a second- or third-degree polynomial equation pattern, in a frequency domain plot. RASIR uses matching of different categories of target objects using a sampling model pattern matching function (PMF) method, where each sampling model is matched towards a set of pre-existing sampling models in a sampling model database function called CCFRP. As RASIR may detect an object based on its RADAR signature sampling model. Such a detection, recognition, and classification of target objects are at least distance invariant as each object will return a reflection that is unique for its non-linear frequency dependent response signal. The response signal strength and polarity may depend on the RCS silhouette presented by the target object. Hence, RASIR offers in most situations a stable system for recognition of small objects and volumes independent, that may be correlated with a relative aspect angle, RCS, and rotation of the target object in mind.

The PMF matches both components relating to a transmitted ITS and a received IRS, towards multiple reference versions of ITS and IRS stored as pairs in CCFRP, under the names RTIS and RIRS. When a matching RTIS and RIRS pair is found, RASIR outputs the RADAR information to an interface as a RASIR Target object Localization and Characteristics (RASIR TLC) datagram to be used in a surveillance system, a target tracking system, or by any external system that makes use of RADAR data information and tracked, localized, and classified target objects.

A special form of target objects is the Wideband Chaos Generating Material (WCGM) 30 that are specially designed as a metamaterial 31 design or chosen by their natural 32 characteristic way of reflecting millimeter RADAR waves. WCGM generates new RADAR scatters reflections usually having a frequency spectrum that differs from the TIS frequency spectrum. Reasons for new frequencies in the WCGM reflections are, millimeter RADAR resonance in the material or material composition chosen, micros structure of surface and structure of material especially when the WCGM is made of a metamaterial, reflections in structure and multiple layers of the material may cause a signal interference phenomenon that leads to new wider range of wavelengths being created, just to mention a few mechanisms why new frequencies may be generated. Metamaterial may be designed as one-way and two-way frequency signal transformers.

Normally signal interference is problematic for RADAR systems. RASIR is contrary to traditional art, making use of interference signals generated by Wideband Chaos Generating Materials (WCGM), as a way to:
a) relay information to objects normally hidden for millimeter RADAR:s due to the fact that millimeter RADAR:s does not penetrate most surfaces, nor the human body beyond the upper skin layers;
b) increase the bandwidth of millimeter RADAR signals, transmitting ITS signals; where the bandwidth is both may be extended beyond the TIS frequency spectrum, and may also fill in and smoothen the TIS signal when the TIS signal consists of only distinct narrow frequencies signals;
c) a third aspect is that Wideband Chaos Generating Materials (WCGM) may act as a signal relay even for signals being reflected from another target object and back towards the RADAR Rx Receiver.
d) a fourth aspect is that Wideband Chaos Generating Materials (WCGM) transforming signals into wider range of wavelengths makes it possible to interrogate the human body internal organs, especially for water contents and for organs demonstrating TOwNLF frequency response behavior;
e) a fifth aspect of the WCGM is that it may when present contribute to and enhance positioning and localization precision, when the locating target objects receives a, preferably but not necessarily predicted, wideband signal from the WCGM.

Details for sub-functions and the RASIR sub-system 1 will be further explained as follows. On a top-level, the RASIR super system 4 is making use of a RASIR system 1 integrated with on a RADAR system 2, preferably a Synthetic Aperture RADAR (SAR) with transceiver antenna arrangement. The RASIR super system further integrates all types of target objects possible in an environment 3 with in the coverage of the millimeter RADAR 2 integrated, such as all types of target objects 22-32, including Wideband Chaos Generating Materials (WCGM) 30-32. Although a RASIR super system would be able to interrogate all types of target objects, most situation benefits from customization and adaption of target objects being used of their actual applications, requirements, and system use.

The RASIR super 4 system's RASIR system 1 matches sampling models for target object 22-29 and Wideband Chaos Generating Material (WCGM) 30-32 by matching alignment of non-linear frequency dependent response function models RTIS 37 and RIRS 38 pairs for target objects and Wideband Chaos Generating Material (WCGM), recorded in the CCFRP 12, and offers RASIR TLC 13 datagram via a RASIR Interface (RASIR IF) 14. This allows an external system making use to RASIR IF and RASIR TLC, to reach into CCFRP to fetch more detail about known objects, and even the location of target objects, and target objects recognized to be of the same type of target object category. RASIR IF even lets an external system request information about recognition patterns such as pre-recorded and analyzed sampling models for target object matching. Likewise, an external system may install new patterns for recognition of sampling models as RTIS and RIRS pairs, with attached data about the type of object being recognized.

Applications exists for object tracking, logistic systems, tracking of dynamic machines and self-propelling machines requiring ego-motion sensors, for medical use, for diaper surveillance in a care center, as a mean to measure 3D objects, and more applications requiring tracking, classification, and recognition of objects.

### Second Embodiment—RASIR System Incorporating a Millimeter RADAR Sub-System

FIG. 1 shows a second embodiment of the invention in the form of the RASIR system 1. The RASIR System is introduced as a new RADAR system. RASIR can detect and analyze target objects based on their frequency response sampling model when interrogated with different RADAR frequencies, pulse forms, Chirps, and repeated interrogation signals, as a method to recognize target objects based on material composition and opportunistic use of surrounding environment and signal interference. The system is especially suited for recognition of target objects and RADAR tags demonstrating a non-linear frequency dependent response signal, by matching RADAR signal response sampling model for sampling model alignment between reference interrogation signals and signatures with interrogation signals transmitted with related received interrogation signatures.

In a second embodiment of the invention, the RASIR system 1 incorporates a millimeter RADAR system 2 for interrogation, detection, localization, and categorization of target objects, as follows.

To generate and control how properties, waveform, and frequency of the RADAR signal, the RASIR system comprises a Transmitter Interrogation Signal Generator (TISG) 6, configured to generate a Transmitter Interrogation Signatures (TIS) 7. TIS defines how the sub system, the RADAR 2 is to generate its waveforms, Chirps, frequency sweeps using its Waveform Generator (WG) function, to generate a RADAR signal 33 is to be transmitted from its Tx Transmitter (Tx) 16, and Tx Antenna 17, towards an environment 3 covered by the RADAR antenna signals, containing target objects 22-32 having various properties such as RADAR signature, attenuation, reflectance, and resonance profiles.

The RADAR signal as defines and specified by the TIS 7 data record, reaches a target object 22-32, and is at least partially reflected to the RASIR RADAR 2 Rx antenna 18.

When the RADAR's 2 Rx antenna receives a waveform 34, containing information corresponding to an Interrogation Receiver Signature (IRS) 9 although not yet in the form of an IRS datagram; the RADAR 2 then relays the Rx signal 34, from Rx Antenna 18 to an Rx Receiver (Rx) 19 which transforms electrical signals representing the IRS into digital sampling model data. The term IRS here refers to the information transferred as an electromagnetic wave, digital signals in the RADAR 2 and RASIR system 1, and later as the IRS datagram 9 prepared for later signal sampling model pattern matching in the PMF 10 and CCFRP 12.

The Rx Receiver then transfers the IRS digital sampled signal to an Tx Rx Mixer 20 that receives both a Tx 16 signal representing the TIS 7 and a Rx 19 representing IRS 9 signal signature for amplification and additive combination into a Tx Rx Mixer 20 signal. The output from the Tx Rx Mixer is then forwarded to a Target Object Position Time-of-Flight (TO POS TOF) 21 classic RADAR target tracking and RADAR analysis function to identify location, direction to, distance to, first time-of-flight (TOF) reflection and volume of a Target Object being search for and detected. The Tx Rx Mixer also transfers its results in combination with the output from TO POS TOF 21 to a Fast Fourier Transformation (FFT) signal transformer 8.

The process to transmit TIS and receive IRS signals are repeated several times, preferably 128-256 times, and sampled into a TIS and IRS signal representing for example 128-256 signals sampled for improved statistical certainty and information concentration.

The FFT 8 receives the Tx Rx Mixer 20 signal, and in combination with the isolated target object position time of flight TO POS TOF 21 as a signal from preferably a MIMO antenna or SISO antenna 17, 18 configurations.

Then FFT 8 then samples the Mixer signal to generate a data sample expressing energy (voltage, gain, number of signal occurrences) distribution over a frequency plane for further processing and generates an Interrogation Response Signature (IRS) 9 that expresses at least a signal intensity as a function of frequency, that is the FFT transform a signal from a time domain to a frequency domain, as a digital or software-based signal.

RASIR's 14 Pattern Matching Function (PMF) 10, then receives a time correlated TIS (7) and IRS (9), where the TIS and IRS mainly represents sampling data in a frequency domain, but the TIS and IRS may also carry information in the time domain, and sampling data about signal polarization, and signal phase, as well as location information carried from the TO POS TOF 21 processing, which lets RASIR 1 separate TIS and IRS pairs relating to a target object found based on a first reflection as calculated by the TO POS TOF 21, thus making it possible to isolate signal interrogation and processing to just one target object at the time, or to two or just a few <10 target objects 22-32 interacting by interference signals 25 and 26 for example by the use of Wideband Chaos Generating Material (WCGM) 30-32 in an environment 3.

The PMF 10, then tries to find and estimate matching pairs of the TIS 7 and IRS 9 received, with pairs of earlier recorded or defined reference transmitter interrogation signal (RTIS) 37 and reference interrogation receiver signature (RIRS) 38 stored in a Catalogue of Characteristic Frequency Response Patterns (CCFRP) 12 database function. CCFRP may be implemented as a relational database, a network database, a knowledge database, or even as neural network, preferably a Convolutional Neural Network (CNN) which in combination with a Deep Learning Neural Network (DLNN) may allow the CCFRP to perform a part of PMF's matching operation and support automatic recording and learning of new discovered target objects with unique TIS and IRS, to be recorded as new RTIS and RIRS in the CCFRP database function. The PMF may use a variety of matching methods, especially adapted to match target objects demonstrating a non-linear frequency dependent reflected signal intensity.

A first aspect of the PMF is that it is no just matching a single TIS and IRS towards RTIS and RIRS candidates in CCFRP, but it may match multiple TIS with IRS representing a target object being interrogated, where the TIS signals may comprise Chirp of different shapes, narrow frequency spikes, frequency swops, random frequency sweeps in a frequency window, where multiple TIS may use different central frequencies, which each results in related IRS sampling models from the target object being matched. For a target object to be perfectly matched, it is required that all, or at least a sufficient number of its TIS to IRS pairs matches similar reference RTIS to RIRS pairs in CCFRP 12.

A second aspect of the PMF is that matches not just single frequencies of a sampling model, but that it uses its knowledge about the target object's signal response signature from transmitted TIS to received IRS, to match for non-linear frequency dependent signal response patterns such as described by first degree polynomial equation, second-degree polynomial equation, third-degree polynomial equations, while using pattern matching alignment methods to find minimal error and deviations between matched signals TIS-IRS and RTIS-RIRS pairs.

Prior art RADAR solutions have been found to just sample signal frequencies at certain narrow frequency bands and then calculating a binary number representing occurrence of a frequency at each narrow frequency band, and then generating an identity of a target object, referred to as a fingerprint. As seen, the RASIR PMF is far mor sophisticated as it uses knowledge about all types of target objects with related signal signature response patterns. This lets RASIR perform a more reliable matching operation than prior art, as all information sources available are used, both from actual target object as a signal response, and from earlier recorded single or multiple reference target objects stored with RTIS and RIRS sampling model signature data. As RASIR is also able to learn new signal sampling models from newly discovered target objects, and from actual target object being interrogated, this makes RASIR able to improve and adapt its pattern matching function's (PMF) performance during operation.

The CCFRP 12 then returns a look-up index, or indexes, of best matched previously recorded reference RTIS 37 and RIRS 38 with attached signal characteristics in CCFRP 12. If no match is found according to programmable alignment requirements to be considered as a matching operation, then assuming the system is set in a learning mode 60, the CCFRP may record and learn the actual found TIS and IRS pairs' sampling models which then are stored as new RTIS and RIRS pairs in CCFRP for future references.

Once a matching target object sampling model is found in the CCFRP and matched as a best candidate, or candidates, then the PMF 10 compiles a Target Localization and Categorization (TLC) 13 data record that describes the target object's location relative to the RADAR antenna arrangement 17, 18, and categorization data such as the TO's look-up index identity in the CCFRP 12 database, the state of the TO 22 in the event that the TO 22-25, 28 represent IRS variations due to a physical state, such as variations in concentration of a sugar-water solution, temperature, shielding as for a Shielded Target Object (STO) 27, bending, and metamaterial 31 changes in structure.

As a result, the RASIR Interface 14, can offer TLC data 13 and CCFRP 12 data access to RTIS 37 and RIRS 38 for an external or integrated system, application, process, a simultaneously localization and mapping (SLAM) system, a RADAR console or supervision central, a vehicle anti-collision system, medical surveillance, caretaker service monitoring system, or any other system consuming at least one out of: target object localization position and categorization 13 and target object's 22 state data.

Further in the second embodiment of the invention, the millimeter RADAR system 2 comprises a Synthetic Aperture (SAR) RADAR, equipped with a Multiple Input Multiple Output (MIMO) antenna arrangement. This lets the RADAR extract information from a single target object 22-29, optionally together with related Wideband Chaos Generating Material 30-32, to let the system interrogate a limited volume of the environment 3 in isolation from target objects and signal sources outside the interrogated volume.

As a result, this lets RASIR 1 interrogate scenarios where a single target object is interrogated alone, and in combination with a target object of Wideband Chaos Generating Material (WCGM) type 30-32 where signal paths and interference signals from and to the WCGM can be analyzed, for matching signal components of matched sampling models in CCFRP. Once a certain type of target object, has been determined, categorized, and located, including the WCGM types; then the RASIR system can make use of reference information in CCFRP to remove signal components from the recognized target objects, to let RASIR:s PMF and CCFRP extract more signal components remaining from target objects and Wideband Chaos Generating Material (WCGM) in the sub-volume of the environment 3. As soon as the target object together with a related Wideband Chaos Generating Material (WCGM) have been mapped in CCFRP and made available via RASIR TLC 13, and the RASIR IF interface 14; then the RASIR system 1's RADAR, preferably a SAR system 2, may extend or move the sub-volume being interrogated by the RASIR system, and make use of any discovered target object 22-32, including any impact on the RADAR system from any recognized Wideband Chaos Generating Material (WCGM), MMIO, or NO 30-32.

Further the second embodiment, the millimeter RADAR system RASIR 1 comprises a RASIR Control System (RASIR CS) 5 for execution control and resource allocation of other functions of the RASIR system. The RASISR CS 5 controls an interactive interpolation process for improved precision of target object position, location, and signature matching by performing the steps to:
a) match TIS 7 and IRS 9 pairs with corresponding RTIS 37 and RIRS 38 pairs in CCFRP 12,
b) select a new TIS candidate by analyzing matched RTIS and RIRS candidates in CCFRP, and
c) to select the new TIS candidate from a RTIS among pairs of RTIS and RIRS in CCFRP, to find the most differentiating RTIS that would generate the most differentiating RIRS signal signature sampling model,
d) use TISG 6, or a waveform generator, select and generate a new TIS 7, expected to result in a matching of the most differentiating RTIS 37 signature model candidate in CCFRP,
e) interrogate a target object 22-32, to generate a new IRS 9 with a matching existing RIRS 38 in CCFRP 12,
f) and then repeat the above steps until a matching RIRS 38 is identified with matching look-up index in CCFRP 12, and
g) to transmit a target object localization and characteristics TLC 13 data record to an interface, for integration with a RADAR data consuming application or system.

Mentioned configuration and steps lets the RASIR incrementally identify a best fitting reference target object description in the form of RTIS and RIRS pairs in CCFRP, by generating the differentiating TIS for interrogation when multiple matching candidates exists in CCFRP.

In a second embodiment, the Pattern matching function (PMF) 10 and Catalogue of Characteristic Frequency Response Patterns (CCFRP) 12 are configured to provide sampling signature model matching in PMF and a look-up database CCFRP that are essential for the matching and recognition function for the RASIR System embodiments.

The signal Pattern Matching Function (PMF) 10, is responsible for matching signal characteristics and sampling models of pairs of TIS 7 and IRS 9 with similar reference pairs reference transmitter interrogation signals (RTIS) 37 and reference interrogation receiver signatures (RIRS) 38 stored in a shared signal signature matching database CCFRS (12). As such then CCFRP and PMF performs the following functions:

CCFRS (12) is responsible for storing and retrieval of data records holding pairs of RTIS 37 and RIRP 38, where the data records also may store:
i) signal matching sampling model signatures, signal characteristics, matching deviations allowed, preferred method of interrogation such as Chirp shape or narrow frequency scanning method, target object position, target object classification, target object classification description, and combinations,

The signal matching function, the PMF 10, is responsible for finding a best alignment between RASIR tag's or target object's actual TIS 7 and IRS 9 pairs, and previously stored RTIS 37 and RIRS 38 pairs, stored in CCFRP according to a specified signal matching method.

The signal matching method for the PMF 10 may be based on:
a) at least square matching method to find best alignment between the sampling models of TIS and RTIS candidates in CCFRP,
b) at least square matching method to find best alignment between the sampling models of IRS and RIRS candidates in CCFRP,
c) a cross-correlation method to identify best signal sampling model match,
d) an auto correlation method to identify best signal sampling model match,
e) a Convolutional Neural Network (CNN) to match sampling model signatures with matching data, and optionally a deep learning neural network (DLNN) as a self-learning signature sampling model matching function enabled if the system is in a learning mode 60, or
f) a search method that tests received target object TIS, IRS pairs with reference TIS and reference IRS pairs in CCFRP, comprising at least one search strategy as:
   i. random search,
   ii. consecutive search and sampling model matching top-down thru the CCFRP data records,
   iii. reverse sampling model matching,
   iv. matching parameters of a second degree or higher degree equations describing mathematical shapes of each sampling model,
   v. digital signal processor (DSP) based matching of sampling signals,
   vi. matching sample models in fixed windows 202, 212, or as rolling windows following a time frame, and
   vii. neural network-based sampling model matching where the whole tree of patterns used are represented in the CCFRP.

The PMF 10 and CCFRP 12, may record new Target Object's 22-32 signal signature sampling model patterns in situations based on related TIS and IRS sampling models that cannot be found in in the CCFRP database, by following the following steps:
a) detect when Target Object's 20-32 TIS 7 and IRS 9 sampling models cannot be matched with any RTIS 37 and RIRS 38 by the PMF 10 in the CCFRP database,
b) assert that the system is in a learning mode 60 before commencing to record and learn a new Target Object. In case learning mode 60 is not enabled, then the procedure will end at this step,
c) in CCFRP generate a new look-up index pointing at a new data record in CCFRP and insert Target Object's 20-32 characteristic sample model data where the TIS will be inserted as a new RTIS, and the IRS will be inserted as a RIRS with sampling models in the CCFRP database, to make the target object recognizable, matchable or comparable by PMF and retrievable from CCFRP.

This lets CCFRP record and learn new then recognizable target objects. Similar methods may be used to ensure that old data records in CCFRP gets disposed. Some target object types, and sampling model signatures may have to be stored permanently in CCFRP, while others may be temporary saved for a short time.

In the third embodiment, a method for the PMF 10 and CCFRP 12 for matching of target object sampling models using alternative matching dimensions may include at least two out of the following methods to match TIS and IRS pairs to RTIS and RIRS pairs in CCFRP:
g) matching of sampling models in the frequency domain for matching of sampled signal magnitude for each frequency,
h) matching of sampling models in the frequency domain for matching of sampled signal magnitude for shifted frequencies to match signals with doppler effect,
i) matching of sampling models in the time model using RADAR pattern matching of time of flight (TOF) chirp patterns in a time domain,
j) matching of sampling models for phase shifts,
k) matching of sampling models in a frequency-polarization domain,
l) matching of sampling models in the frequency domain for matching of sampled relative signal magnitude for each frequency for best curve alignment independent of magnitude,
m) matching of sampling models to match for RADAR Cross Section (RCS),
n) matching of sampling models in the frequency domain for matching frequency patterns of frequency shifted signals, for recognition of frequency shifted signals from metamaterials and Wideband Chaos Generating Materials (WCGM).

The mentioned methods specify multi-dimensional strategies and algorithms to be used as candidates to ensure a matching pair of sampling models. For clarity, it should be stated that RASIR is mainly using the Time-of-Flight (TOF) method as being used by most RADAR systems, and then matching of preferably non-linear frequency dependent signal magnitudes in a frequency plane, between TIS and RTIS, and then between IRS and RIRS, for each sample of TIS to IRS signal-response pairs. Normally, these comparisons cover about 128-256 Tx signals sampled as a TIS and similar 128-256 responses sampled as IRS.

### Dynamic Behavior and Method Used by the RASIR System 1

To let the RASIR system 1 interrogate, localize, and categorize of target objects 22-32 it has to apply certain methods, methods that are usually orchestrated by RASIR's control system (RASIR CS) 5 to perform millimeter RADAR based signal interrogation of target objects (22-32), and also to interrogate target objects via and using an Wideband Chaos Generating Material (30-32), and to analyze reflection signals from target objects and the Wideband Chaos Generating Material, to detect, localize the position of target objects using RADAR principles, and to categorize target objects by interrogating target object's to determine the target objects frequency dependent RADAR reflection and signature response sampling model, in at least a frequency domain, to recognize target objects having a non-linear transmitter interrogation signal frequency dependent signal response signature, according to the methods presented as follows.

The methods presented as follows can be applied to realize the first, second and third embodiments.

In some embodiments the RASIR system 1 integrated with a millimeter RADAR 2 is using the following method to detect a target object (TO) 22-32, localize the TO position, to interrogate the TO, and to categorize and recognize a TO's frequency response signature sampling model, find a best matching reference target object described by reference sampling models RTIS and RIRS.

To interrogate Target Objects 22-32, the RASIR system 1 follows the steps where the RASIR System:
o) Interrogates a Target Object 22-32 by transmitting a Transmitter Interrogation Signal (TIS) 7 RADAR signal from a RADAR Tx 16 antenna 17, oriented towards a space expected to comprise a target object 22-32,
p) Receives a Rx 19 receiver signal originating from the target object 22-32 and received via an Rx antenna 34,
q) Mixes 20 the RADAR signal 16, that is based on an interrogation signature TIS 7 and the received Rx 19 receiver signal,
r) Transforms the RADAR signal from a time domain into a frequency domain using a Fast Fourier Transform (FFT) 8 sampling the RADAR sample model reflected from the target object, to generate an Interrogation Receiver Signature (IRS) 9 signal for further analysis associated with the TIS 7 signal transmitted for the interrogation,
s) Performs a pattern matching of a first pair comprising the TIS 7 and the Interrogation Response Signal (IRS) 9 for the target object 22-32 being interrogated, representing a first sampling model, with a second pair comprises a Reference Transmitter Interrogation Signal (RTIS) 37 and a Reference Interrogation Response Signal (RIRS) 38 where the second pair represents a second sampling model, and the RASIR stores the second pair RTIS and RIRS a Catalogue of Characteristic Frequency Response Patterns (CCFRS) 12 database,
t) Returns at least one look-up index to a matching, or new created, data record in CCFRS holding a second pair of RTIS and RIRS,
u) Updates a target object, location, and categorization (TLC) data record, and transfers the TLC data record to an external application, via an interface RASIR IF 14 for RADAR data consumption.

To record and learn to recognize new target objects, the RASIR system 1 follows the following steps:
v) If no matching second pair RTIS and RIRS can be identified in the CCFRS, during normal RADAR based interrogation of target object (22-32), and the system is set in a learning mode 60, then the RASIR system creates and inserts a new data record in the CCFRS 12 database, and
w) saves the first pair TIS and IRS, as a new second RTIS and RIRS pair in CCFRS as a new second sampling model, for later referencing and pattern matching.

To detect, determine location of, recognize, and to categorize of target objects 22-32 the RASIR system 1 uses its Pattern Matching Function (PMF) 10 to perform matching of new RADAR signals with existing signal definitions in the CCFRP 12, by comparing and matching signals sampling model pairs of TIS and IRS with corresponding pairs RTIS and RIRS stored in CCFRP using matching:
x) by finding the least square error match in the signal frequency domain comparing Frequency-Amplitude according to signature sampling model of the TIS 7 and IRS 9 pairs and the RTIS 37 and RIRS 38 stored in the CCFRP 12,
y) of doppler shift sampling models,
z) of polarization sampling models,
aa) of frequency resonance spectrum intensity sampling models,
bb) of frequency resonance spectrum phase sampling models,
cc) of frequency resonance spectrum matching of frequency resonance spectrum phase sampling models,
dd) by finding the least square error in the pairs ITS, IRS, in relation to the pairs RTIS, RIRS signals time domain representation,
ee) by comparing non-linear frequency dependent response signals sampling model patterns, where the frequency response signature sampling model in an IRS 9 reflected from at target object 20, 26 hit by a specific TIS 7 aligns with a third-degree polynomial equation function signature sampling model, and where extracted equation parameters are matched with corresponding parameters for existing RTIS 37, and RIRS 38 pairs, stored in CCFRP 12,
ff) of sampling models by determining and analyzing auto correlation between the TIS 7 and the RTIS 37, and between the IRS 9 and the RIRS 38,
gg) of sampling models by determining and analyzing cross correlation between the TIS 7 and the RTIS 37, and between the IRS 9 and the RIRS 38,
hh) of sampling models by determining and analyzing by using any other correlation method between the TIS 7 and the RTIS 37, and between the IRS 9 and the RIRS 38,
ii) implemented by a Digital Signal Processor (DSP), or a Field Gate Programmable Array (FPGA) programmed for matching of signal sample models, or
jj) using a neural network such as a Convolutional Neural Network (CNN) configured for matching of signal sample models.

This lets RASIR perform matching of pattern in multiple dimensions.

Still the main method for pattern matching for RASIR is to perform matching of sampling models from TIS and IRS compared with RTIS and RIRS in CCFRP, in a frequency domain, where the sampling models are compared and evaluated for alignment and pattern matching errors, when comparing signal magnitudes for each frequency in the RTIS and RIRS in CCFRP.

To interrogate target objects 22-29 via Wideband Chaos Generating Material (WCGM) 30-32, the RASIR system may perform pattern matching in a multi-path scenario where the interrogation signal TIS first reaches a Wideband Chaos Generating Material (WCGM) 30-32 and reflects as an RTIS towards the target object 22-29 which then reflects to the RASIR RADAR 2, by using a pattern matching function PMF 10 to find a best signal signature match of the first and second sampling models that:
kk) matches of an Altered Transmitter Interrogation Signal (ATIS), that is a TIS transformed into an ATIS by a Wideband Chaos Generating Material (WCGM) 30-32, with an RTIS sampling model stored in CCFTS where the ATIS 36 and RTIS correlate, and then
ll) matches a second sampling model comprising the IRS and the RIRS in CCFRS, to match a target object based on remote interference signal ATIS from a Wideband Chaos Generating Material (WCGM) 30-32, to match a target object via a TIS signal reflected by the Wideband Chaos Generating Material (WCGM) to a target object and back to the RASIR RADAR 2.

The RASIR System 1 can be equipped with a RADAR system 2 limited to a Single Input Single Output (SISO) transceiver antenna configuration 17, 18. With this SISO antenna configuration, the RASIR System may still use TIS to interrogate target objects, Wideband Chaos Generating Material (WCGM), and target objects via Wideband Chaos Generating Material (WCGM), and back to the RADAR, as the PMF can still perform pattern matching of sampling models retrieved from RADAR environment 3 and target objects 22-29, as well as Wideband Chaos Generating Material (WCGM) 30-32. Time to flight TOF based matching is still available for a RASIR System with SISO RADAR, but separation of direction target objects and lobe in general cannot be realized without special signal processing algorithms.

The RASIR System 1 can be configured with a RADAR 2 equipped with a Multiple Input Multiple Output (MIMO) antenna transceiver, or a Synthetic Aperture Radar (SAR) with MIMO capabilities. In such an equipment configuration, RASIR could make use of the RADAR's capability to for each target object 22-29 and Wideband Chaos Generating Material (WCGM) 30-32, identify a RADAR sector and distance, defining a volume, form where the RADAR system could separate and isolate signals for such a volume, thus making it easier for the RASIR system to analyze complicated signal multi-path situations.

Also, RASIR can, the ability to direct a RADAR signal towards mentioned volume, and to shape the RADAR lobe for best configuration, and for a RADAR capable of using multiple lobes as simultaneous separate RADAR interrogation lobes. In such SAR and MIMO configuration, RASIR can select to:
mm) first calculate (20) a Time of Flight (TOF) distance and antenna relative position of the target object (22-32), and
nn) then with the help from the RADAR system 2 calculate a TOF distance and direction compensated RADAR reflection from the target object 22-32, to let the RASIR system process each target object separately, or in combination with specific Wideband Chaos Generating Material (WCGM) (30-32), for example based on a distance, direction, or three-dimensional structure located in relation to the RADAR antenna configuration 17, 18.

As the RASIR system 2 interrogates its RADAR coverage environment 3 for a TO 22-29, WCGM 30-32 or both, and then searches for previous known TO:s and WCGM:s recorded, specified, or recognizable for an a TO or WCGM candidates match, the RASIR system and its Pattern Matching Function (PMF) 10, needs as method and function to store previous specified, learnt, and recently recognized TO and WCGM RADAR signal signature sampling models. This function for storage and retrieval is the database function Catalogue of Characteristic Frequency Response Patterns (CCFRP), which may hold typically data records pairs of RTIS 37 and RIRS 38. CCFRP may also hold other characteristic patterns of signal signature sampling models that are not frequency based, such as polarization, phase shift, Time-Of-Flight (TOF) based (TOF) patterns, to mention a few parameters for pattern matching of signals and signatures.

In operation, the RASIR system 1 and its PMF 10 uses, search for or creates a look-up index in CCFRP to a data record, letting the RASIR system, using the look-up index in CCFRP to, extract from, and transfer information to the RASIR TLC 13 data record, and the PMF 10, where the CCFRP transferred information comprises combinations of:
a) an uniquely identity of associated target object type 20-32,
b) extract information regarding matched pairs of reference RTIS 37 and RIRS 38 sampling model characteristics,
c) extract data values associated with the look-up index such as:
   state of target object, Brix value of sugar water solution, amount of water or fluid, signature sampling model having frequency dependent response signal (linear, non-linear, second degree polynomial equation function sampling model, or second degree polynomial equation function sampling model), target object identity, target object material property, target object data, location (position, direction of travel, speed) data, historical data, or any combination.

The RASIR system's 1 pattern matching function (PMF) 10 may find multiple candidates of matching reference target object signatures, expressed as RTIS and RIRS pairs in CCFRP 12 hence the RASIR System may transfer information about these look-up candidates found in CCFRS with a confidence value indicating errors in alignment between matched signal signature pattern models.

Hence, the RASIR system and its PMF 10 together with CCFRP 12 uses a method where multiple look-up indexes and a matching confidence value, are transferred to the TLC data record, to support matching of a target object with multiple look-up indexes, and sampling models in CCFRS 12 defined by pairs of RTIS 37 and RIRS 38 sampling models.

When the RASIR System 1 performs interrogation of target objects 22-29, it is repeatedly transmitting TIS based RADAR signals, resulting in received IRS, in the range of 10-500 times, before it concludes that the PMF has found a matching RTIS and RIRS in CCFRP. If it has not found a matching signal sampling model in CCFRP, or if it has found multiple candidates in CCFRP, then the RASIR Control System (RASIR CS) 5 or similar control loop can select to, interrogate the target object using an alternative TIS to interrogate and fetch a potentially more significant IRS that matches a target object, or an IRS that clearly indicates a unique target object response signal sampling model, that better indicates the best match out of RTIS and RIRS pair data records in CCFRP.

To support a better TIS based on what TIS that would generate the best distinguishing IRS, the RASIR system my follow the following procedure that may be used in situation where RASIR needs more information to determine proper matching of sampling models for the actual target object, or Wideband Chaos Generating Material (WCGM) being interrogated.

In a situation where no matching or double matching of sampling models occurs:
a) generate a TIS 7 that maximize the uniqueness of an IRS 9 reflected by set of target objects 22-32 being searched for in range for the millimeter RADAR 2,
b) or when searching for a specific target object (212-32) type, or target objects, having a reference RTIS 37 and RIRS 38 signal characteristic signal response, from closest a matching TIS signal sampling model in CCFRP
c) select among RTIS and RIRS pairs, a new TIS (7) signal definition wherein corresponding RIRS (38) sampling models are as unique as possible among other target object in range for the millimeter RADAR,
d) to allow for efficient identification of relevant target object (22-32), and a higher precision in target object localization and categorization (21).

According to a first aspect, when the RASIR system's RADAR is transmitting its electromagnetic millimeter RADAR wave representing a TIS interrogation signal, the RASIR System, first transmits a TIS 7 represented by a millimeter RADAR signal 33, having a center frequency between 4 GHz and 77 GHz preferably 24 GHz and 77 GHz, and a sub-bandwidth between 250 MHz 4000 MHz and preferably 1700 MHz.

According to a second aspect, when the RASIR system's RADAR is transmitting its electromagnetic millimeter RADAR wave representing a TIS interrogation signal, the RASIR System, first transmits a TIS 7 represented by a millimeter RADAR signal 33, having a center frequency between 16 GHz and 115 GHz preferably 70, and a sub-bandwidth between 0.25 MHz and 500 MHz and preferably 200 MHz.

According to a third aspect, when the RASIR system's RADAR is transmitting its electromagnetic millimeter RADAR wave representing a TIS interrogation signal, the RASIR System, first transmits a TIS 7 represented by a millimeter RADAR signal 33, having a center frequency between 20 GHz and 30 GHz preferably 24 GHz, and a sub-bandwidth between 100 MHz 400 MHz and preferably 200 MHz.

When the RASIR system's RADAR is interrogating target objects 22-29 using or being under influence of longer wavelengths signals from a Wideband Chaos Generating Material (WCGM) 30-32, or interrogating Wideband Chaos Generating Material (WCGM) alone;
a) then the RASIR system's RADAR transmits its electromagnetic millimeter RADAR wave representing a TIS interrogation signal on a narrow band,
b) while as a result from the use of Wideband Chaos Generating Material (WCGM) which reflects and generates RADAR waves of wider range of wavelengths, it becomes necessary to be able to receive a wider frequency range and longer RADAR waves than millimeter RADAR waves,

Therefore, the RASIR system's 1, RADAR transceiver is configured to transmit interrogation signal representation of TIS 7 as a millimeter RADAR signal 33 having a center frequency between 4 and 77 GHz preferably 24 and 77 GHz, and a sub-bandwidth between 250 and 4000 MHz preferably 1700 MHz.

Alternatively the RASIR system's 1, RADAR transceiver is configured with a RADAR transceiver with:
a) a Tx antenna 17 that transmits a TIS 7 defined RADAR signal 33 at a center frequency between 4 GHz to 77 GHz with a sub-bandwidth of 250 to 4000 MHz,
b) a Rx antenna 18 with a Rx Receiver 19 that receives RADAR reflection signals 34 at a center frequency between 1 GHz to 124 GHz with a sub-bandwidth of 0.1 to 4000 MHz, preferably 1700 MHz, and
c) a RADAR 2 that samples a sampling model, representing an Interrogation Receiver Signatures IRS 9;
d) to let RASIR receive IRS and Altered Transmitter Interrogation Signatures (ATIS) wideband RADAR signals 34, 36 from target objects (TO) and Wideband Chaos Generating Material (WCGM) 30 metamaterials 31, natural occurring target objects 32, target objects inside body (TOIB) 29, and shielded target objects (STO) 27.

To improve precision for the RASIS System 1 concerning its ability to measure exact distances and direction towards target objects including meta materials being interrogated, the RASIR system may use its CCFRP database to improve its measuring precision, by selecting a best TIS 7 and matching for an expected IRS 9. The RASIR system's RADAR's time of flight (TOF) measurements as well as angle of arrival measurements may benefit in precision from the selection of a TIS that would generate the best response IRS. In a MIMO and SAR RADAR, angle of arrival calculations could also benefit from knowing which IRS to expect based on prior knowledge from the target object being interrogated, and any reference information in CCFRP.

The RASIR RADAR 2 can make Time of Flight measurements and angle of arrival response signal signatures in the timed domain when analyzing the IRS received.

Similar effects are possible for precision improvements in when interrogating and localizing target objects, using an FMCW millimeter RADAR 2, at for distance, and angle of arrival calculations. Knowing the expected IRS signature from earlier IRS received from the target object, as well as IRS to be expected according to target object profiles stored in CCFRP, can contribute with information to improve matching of IRS sampling models in a frequency domain. It is known from prior art that different Chirp signal waveforms are efficient for different FMCW RADAR applications.

Also, for the RASIR System's 1 ability to perform signal signature sampling model pattern match inching using its Pattern Matching Function (PMF) 10 and its CCFRP 12 database function, may highly benefit in precision for the PMF:s sampling model matching method, if a specific TIS 7 can be selected, having an expected IRS 9 based on prior knowledge in CCFRP as RTIS 37 and RIRS 38. Therefore, the RASIR System may apply the following method to increase its sampling model pattern matching precision, where the RASIR System:
a) first identifies a Target Object 22-29, including any WCGM 30-32 by performing pattern matching in PMF 10 of the Target Object's 22 non-linear, or linear, frequency dependent Interrogation Response Signature IRS (9), with an existing catalogue of reference RIRS 38 sampling models stored in the Catalogue of Characteristic Frequency Response Patterns (CCFRS) 12;
b) returns a look-up index to a data record in CCFRS that matches the Target Object's, or any RASIR Tag's, IRS 9 when interrogated with a corresponding TIS 7 and RTIS 37;
c) returns a matching score for Target Localization and Categorization (TLC) 13;
d) returns a reference to signal characteristics for reference RTIS 37 and RIRS 38 stored in CCFRS 12,
e) to further let the RADAR make use of a reference to expected reference RTIS 37 and RIRS (38) to be detected, for improved target object localization precision; and
f) adjust the Target Interrogation Signal Generator (TISG) to generate at least one TIS that is based on a RTIS 37 from a previously recognized, or previously stored pattern model signature, for the target object 22 being interrogated, from a set of RTIS 37 and RIRS 38 sampling models,
g) where the TIS is selected among a RTIS 37 results for a better differentiation of reflected IRS 9 for the target object 22, than a previous TIS 7 signal transmitted.

The RASIR System 1 may be used for remote measurement of Target Objects (22-30) demonstrating a remote RADAR 2 readable sensor effect measuring a physical property, such as the:
i) Active Target Object (ATO) 23 which can transfer information by altering its frequency response signature due to a physical input such as temperature change, rotation of an object, exposing, and hiding a target object for RADAR interrogation;
ii) Location Target Object (LTO) 24 which may signal a physical position using an ATO 23;
iii) Target Object for Detection of water-based Solutions (TODS) 25;
iv) (High Frequency Readable Diaper (HFD Diaper) 28;
v) Target Objects Inside a human Body (TOIB) 29;
vi) Metamaterial (MMIO) 31; and
vii) Natural Objects (NO) demonstrating RADAR readable sensor effects 32.

Hence, the RASIR system 1 supports a method to capture target object data, and RASIR system RADAR 2 readable sensor data from target objects data, and then to store information about objects in CCFRP 12, and transfer a RASIR Target Object Location and Characteristics (RASIR TLC) datagram to an external integration interface (RASIR IF); by compiling at least one of the following target object related data as RASIR TLC datagram for the RASIR IF:
a) target object's 22-32 physical state such as closed, open, a temperature, strains, forces, and humidity;
b) target object's 22 chemical properties, such as water sugar concentration;
c) target object's 22 distance to antenna arrangements 17, 18;
d) target object's 22 angle direction as seen from the RADAR antenna arrangements 17, 18;
e) target object's 22 speed and movement direction in relation to the RADAR antenna arrangements 17, 18;
f) a sampling model matching score rating the match between pairs of TIS 7 and IRS 9 with reference pairs RTIS 37 and RIRS 38 stored in CCFRP 12, and the Target Object's 22 reflected signal strength.

The RASIR system 1 supports an interrogation method where Target Objects (TO) 22-32 are interrogated via a Wideband Chaos Generating Material (WCGM) 30-32 according to the following steps where the RASIR system:
a) detects and analyzes a first target object 22-32 being a Wideband Chaos Generating Material (WCGM) according to mentioned RASIR System target object interrogation principles, to learn the characteristic signal response pattern as a sampling model for the WCGM, a function transforming RADAR signals from TIS to IRS, and Altered Transmitter Interrogation Signal (ATIS) 36 RADAR signal representations;
b) uses the WCGM by transmitting a TIS towards the WCGM to let the WCGM transmit an Altered Transmitter Interrogation Signal (ATIS) 36, that is further transmitted from a Wideband Chaos Generating Material (WCGM) 30-32 towards a second target object 22-29 where the WCGM's 30-32 signal transformation sampling model is defined by a RTIS 37 to RIRS 38 in the CCFRS database 12;
c) receives a reflected IRS from the second target object 22-29 as a result from the ATIS 36 transmitted from the WCGM 30-32;
d) performs a pattern matching for the estimated ATIS from the Wideband Chaos Generating Material (WCGM), that interrogates the target object and received IRS from the second target object with existing pairs RTIS 37 and RIRS 38 in the CCFRS 12 database, to match, to identify and to categorize the second target object's 22-29 sampling model as stored in CCFRS 12.

RASIR system 1, also supports an interrogation method where Target Objects (TO) 22-32 are interrogated via an Wideband Chaos Generating Material (WCGM) 30-32, in which the Wideband Chaos Generating Material (WCGM) then relays the interrogation results back to the RASIR system's RADAR 2, according to the following steps where the RASIR system:
a) detects, interrogates and analyzes a first target object 22-32 of a Wideband Chaos Generating Material WCGM 30-32 type;
b) uses the WCGM by transmitting a TIS towards the WCGM to let the WCGM transmit an Altered Transmitter Interrogation Signal (ATIS) 36, that is further transmitted from an Wideband Chaos Generating Material (WCGM) 30-32 towards a second target object 22-29, where the WCGM's 30-32 signal transformation sampling model is defined by a RTIS 37 to RIRS 38 in the CCFRS database;
c) uses the WCGM 30-32 receive reflected IRS from a second target object 22-29 as an effect of earlier ATIS 36 transmitted from the WCGM 30-32 towards the second target object;
d) uses the WCGM 30-32 to let the WCGM transform the received signal sampling model IRS received from the Target Object into second an IRS transmitted from the WCGM towards the RADAR receiver;
e) receive the second IRS 34 from the WCGM 30-32, as a result from the signal path and reflections from the RADAR Tx antenna 17 to the WCGM 30-32 to the TO 22-29 back to the WCGM 30-32, and back to the RADAR Rx antenna 18;
f) matches signals in the pattern matching function (PMF) 10, analyzing the IRS 9 signal sampling model component from the WCGM to extract the IRS component originating from the TO;
g) matches the ATIS signal and the TO's IRS sampling model with existing sampling models in CCFRS 12 comprising pairs of RTIS 37 and RIRS 38 in the CCFRS database 12, to match, identify and categorize the second target object's 22-29 sampling model as stored in CCFRS; and
h) then transfer the matched target object (TO) data generated as a RASIR TLC datagram 13, to be offered to an external system, an application usage RASIR as described, or a RASIR Radar surveillance system.

### Third Embodiment—System of Target Objects with Wideband Chaos Generating Material (WCGM) for the RASIR System

FIG. 1 also presents a “target objects and model of environment” describing a system of target objects of different sub-classes and behavior for the system in first embodiment and second embodiment, where the special class of target object 22-32 including Wideband Chaos Generating Material (WCGM) 30 with sub-classes metamaterial interference object (MMIO) 31 and natural objects (NO) 32, all demonstrating useful functions for usage as slave RADAR signal sources contributing to the detection and mapping process for recognition, classification, and localization of target objects. This constitutes a third embodiment of the invention.

The third embodiment is based on features of the second embodiment, while expanding with the usage of Wideband Chaos Generating Material (WCGM) as an opportunity for RASIR 1 to discover more information about its environment 3.

Further in a third embodiment, the Pattern Matching Function (PMF) is able to perform pattern matching of sampling models from TIS, IRS, ATIS, RTIS, RIRS in:
a) a frequency domain,
b) a time domain, or
c) a combination of a frequency domain and a time domain, to establish a matching situation between sampling models for:
i) IRS 9 to RIRS 38,
ii) TIS 7 to RTIS 37, and
iii) ATIS 36 to RTIS 37.

In a third embodiment, RASIR 1 uses its millimeter RADAR 1 Tx Antenna 17 transmit a RADAR signal such as a RADAR pulse, a continuous RADAR wave signal, a RADAR Chirp, or a TIS 7 based transmitter interrogation signal TIS 7, 33 towards a Wideband Chaos Generating Material (WCGM) 30-32, which further reflects its emitted characteristic RADAR signal 34 towards the Rx Antenna 18 for further analysis and sampling model pattern matching 10 of the WCGM's signal characteristics combined in an IRS 9, to let the sampling model matching function PMF 10 determine the signal characteristics of the Wideband Chaos Generating Material (WCGM) 30-32 as a special form of target object 22, for use as a slave signal generator for improved interrogation of other target objects 22-29.

The RASIR system 1 uses its millimeter RADAR 2 Tx Antenna 17 to transmit a RADAR signal, a RADAR pulse, a continuous RADAR wave signal, a Chirp, or a TIS 7 based interrogation towards a Wideband Chaos Generating Material (WCGM) 30, MMIO 31, and NO 32. Then the target object 22-29 receives a secondary RADAR signal ATIS 36 from the Wideband Chaos Generating Material (WCGM) 30-32. The target object 22-29 then reflect its characteristic RADAR signal 34 based on its received ATIS 36 signal and transfer the characteristic signal comprising information related to an IRS 9, which is received by the Rx Antenna 18 for further analysis and sampling model matching 10 of the WCGM's signal to target object induced response signal path (33, 36, 34). This lets the Wideband Chaos Generating Material (WCGM) acts as a secondary slave RADAR transmitter generating a TIS 7 like signal, which is the ATIS 36, for improved interrogation of target objects 22-29.

Furthermore according to a third embodiment, the RASIR system 1, further:
a) uses its millimeter RADAR system, and the Tx Antenna 17 to transmit a RADAR signal 33, a RADAR pulse, a continuous RADAR wave signal, or a Chirp according to a TIS 7 for interrogation of an Wideband Chaos Generating Material (WCGM) 30, a Meta-material interference object (MMIO) 31, and/or a Natural object (NO) with 32 non-linear frequency response signature, and another target object 22-29 which receives a secondary RADAR signal ATIS (36) transmitted or reflected from a Wideband Chaos Generating Material (WCGM) 30, MMIO 31, 32, and
c) the target object 22-29 further reflects its characteristic RADAR signal 34 back to the, Wideband Chaos Generating Material (WCGM) 30, 31, 32 which transforms the RADAR signal, and
d) transfers the signal further back to the Rx Antenna 18 for
e) further analysis and sampling model matching 10 of the WCGM's signal to target object induced response signal path 33, 36, 35, 34,
f) and as a result lets the Wideband Chaos Generating Material (WCGM) acts as a secondary slave RADAR transmitter generating a TIS 7 for improved interrogation of target objects 22-29 while relaying the target object's 22-29 signal to the Rx Antenna 34.

This lets the RASIR interrogate target objects via Wideband Chaos Generating Materials (WCGM) and back to the target objects, hence the WCGM acts as a dual directed signal relay station for the RASIR RADAR 2.

During the signal transformations in the Wideband Chaos Generating Material (WCGM) 30, frequencies, phases, and signal intensity, or magnitude are transformed, optionally to and from a frequency band of wider range of wavelengths than the original millimeter band, or within the first transmitted original TIS based RADAR signal. The exact signal transformation model for the Wideband Chaos Generating Material (WCGM), is either discovered by interrogation, or pre-recorded in the CCFRP database and stored as RTIS and RIRS sampling models, describing the signal transformation.

Further in the third embodiment, the RASIR system can use Wideband Chaos Generating Material (WCGM) to reach to and into materials otherwise repelling, absorbing, shielded, or reflecting, a millimeter RADAR signal. One such case is then the RASIR system transmits a TIS 7 defined RADAR signal 33 directed towards Wideband Chaos Generating Material (WCGM) 30-32, where the WCGM at least partially transforms the RADAR Tx signal (33) to a lower frequency spectrum signal ATIS 36, having a longer wave-length, capable to penetrate materials and to interrogate target objects using a more wider frequency spectrum than the TIS, and with an altered signal intensity to transfer the relayed signal ATIS 36 to a target object 22-29, and especially to a Shielded Target Object (STO) 27-29 which can now be reached at the lower frequency spectrum signal 36.

As a result, the STO 27, HFD Diaper 28, TOIB 29, or target object 22-26 can return reflect the ATIS 36 signal, as an altered Rx signal 34 towards the RADAR 2 and RASIR system 1 where the Rx signal will carry wavelengths with wider range of wavelengths than the original millimeter band. An alternative signal path, which may be likely active simultaneously with the mentioned signal path is that that the STO, HFD Diaper, TOIB or other target object 22-32 may reflect its signal as an AIRS 35 signal via an Wideband Chaos Generating Material (WCGM) 30, MMIO, and NO 32, that is then transformed into an IRS 9 described Rx signal 34, sent to RASIR's RADAR receiver 18-21, and further processed by the RASIR subsystem 1.

For the RASIR system according to a third embodiment, the RASIR can interrogate a target object using a TIS defined Tx signal 33 having a narrow frequency spectrum that would potentially miss fine-tuned resonance frequencies from a target object having a narrow resonance frequency, by using an Wideband Chaos Generating Material (WCGM) 30 that creates a noise signal, acting like multi-scattering filter, and as a diffusor filter spreading the interrogation signals in more directions and with a smooth frequency profile. Usage of Wideband Chaos Generating Material (WCGM) near target objects being interrogated, may contribute with a better frequency coverage, and direction coverage, for RADAR Rx signal waves 33 transformed into ATIS 36 signals by the Wideband Chaos Generating Material (WCGM) 30.

Hence, the millimeter RADAR system RASIR 1 can makes use of a Wideband Chaos Generating Material (WCGM), MMIO, or NO 30-32 configured to at least partially transform any received signal RADAR signal 33 into an ATIS 36, where the TIS 7 specifies a narrow sub-band frequency Tx signal 33 having a frequency sub-band between 500 MHz and 4000 MHz preferably 1700 MHz, into wider frequency spectrum carried by the ATIS 36 signal having a frequency between 3 GHz and 78 GHz, thus generating and filling in the frequency gaps between consecutive distinct TIS 7 Tx signal 33 frequencies, when transmitting an interrogation signal 34, 36, acting as a filtered TIS 7 signal 34, 36 for a wide-band interrogation of target objects (22-29). For future applications using new hardware solution, it will be feasible to use frequencies beyond 4 GH<, and sub-bands of frequencies beyond 1.7 GHz, preferably divided by 12 sub-bands or more.

Included in the third embodiment, the millimeter RADAR system RASIR 1 is equipped with a Pattern Matching Function (PMF) 10, differentiates matching of signal contributions in a received Interrogation Response Signal (IRS) 9 based on signals from at least two of the following RADAR signal paths, and location of objects, for situations involving any of the target object types TO 22, ADO 23, LTO 24, TODS 25, TOwNLF 26, STO 27, HFD Diaper 28, TOIB 29, WCGM 30, MMIO 31, and NO 32:
a) Tx to 33 TO to 34 Rx;
b) Tx to 33 WCGM to 34 Rx;
c) Tx to 33 WCGM to 36 TO to (34) Rx; and
d) Tx to 33 WCGM to 36 TO to (35) WCGM to (34) Rx.

That the RASIR system differentiates matching of signal contributions, means that to recognize a specific TO, the RASIR system may require that at least two of mentioned signal paths, received as IRS signals 9 in RASIR, can be needed to assert that the signal sampling models support a matching scenario for the target object, or Wideband Chaos Generating Material 30, MMIO 31, and NO 32 being interrogated. Analysis of mentioned signal paths also contributes with more information to the PMF and CCFRP signal signature sampling model matching methods.

For example, the signal components leading thru multiple nodes as in situations c) and d) can be resolved and understood if situation a) and b) are fully mapped and analyzed. This allows RASIR to transmit TIS signals via WCGM (30-32) with control and deterministic generation of ATIS 36 signals for further interrogation of other TO:s 22-29 including other Wideband Chaos Generating Material 30, MMIO 31, and NO 32

In the third embodiment the RASIR system's 1 millimeter RADAR transmitter 16 has a center frequency between 4 GHz to 77 GHz, and a sub-bandwidth of 250 to 4000 Mhz. This defines the RASIR system 1 as a millimeter wavelength RADAR system.

In the third embodiment the RASIR system's RADAR transmitter 16 and Tx antenna 17 has a center frequency between 7 GHz to 77 GHz, and a sub-bandwidth of 0250 to 4000 MHz, while the Rx antenna 18 and Receiver 19 can receive and capture signal signature sampling models having a frequency between 3 GHz to 78 GHz with a sub-bandwidth of 100 to 4000 MHz, preferably 4000 MHz. This means that RASIR may transmit signals at a narrow bandwidth on the millimeter wavelength RADAR band, while receiving RADAR signals having a wide band with wavelengths well in wider range of the original millimeter band, down to frequencies having wavelengths down to 3 cm.

For the third embodiment where the RASIR system 1 makes use of the Wideband Chaos Generating Material (WCGM) 30-32 of different types. The following expresses a few variants of such Wideband Chaos Generating Material (WCGM) 30 compositions designed, selected and configured for use with the RASIR system 1 to improve the system beyond what is normally possible with a millimeter RADAR 2, such as to make use of WCGM to:
a) transform TIS signals to new and wider range of frequency spectrums,
b) reflect TIS signals to a target object
c) smoothen TIS frequencies to extend signal sidebands
d) transform a TIS into longer wavelengths able to penetrate surfaces normally blocking millimeter RADAR, and
e) create a waveform local to a target object being interrogated using a TIS,
f) fill in gaps of TIS frequencies not reached in case the TIS defines a narrow frequency band, like a frequency spice for interrogation of target objects.

A third embodiment where the Wideband Chaos Generating Material (WCGM) 30, 31, 820, 821 for the RASIR system 1, and RASIR super system 4 is an Wideband Chaos Generating Material (WCGM) 30, 31:
a) designed as a Metamaterial Interference Object (MMIO) 31 designed to reflect and transform millimeter RADAR signals into a frequency spectrum having frequency components with wave lengths longer than 1 millimeter.
b) composed of non-metamaterial or natural object (NO) 32 capable to reflect and transform millimeter RADAR signals into a frequency spectrum having frequency components with wave lengths longer than 1 millimeter.
c) where the Wideband Chaos Generating Material (WCGM) 30 is configured to receive a millimeter RADAR signal at a first signal strength and first frequency spectrum, and to
   emit a RADAR signal at a second signal strength, and a second frequency spectrum wherein the first frequency signal spectrum, and second frequency signal spectrum width differs.
d) where the Wideband Chaos Generating material (WGCM) 30 is configured to transform a RADAR signal, typically
   received from a shielded target object (STO) 27 or a target object 22, and the Wideband Chaos Generating Material (WCGM) 30 is configured to receive a RADAR signal at a third signal strength and frequency spectrum, and transform and emit a millimeter RADAR signal at a fourth signal strength and frequency spectrum, having:
      i. the third signal comprises wavelength components of wavelengths longer than 1 mm of the third signal,
      ii. the Wideband Chaos Generating Material (WCGM) 30, optionally made of metamaterial (MMIO) 31, transforms the third frequency signal into the fourth signal frequency millimeter RADAR spectrum, and
      iii. the emitted fourth frequency contains information from the third signal's frequency spectrum having longer wavelengths than 1 mm.
         Prior Art, Suffering from Reduced SNR and Disturbing Interference Signals from WCGM

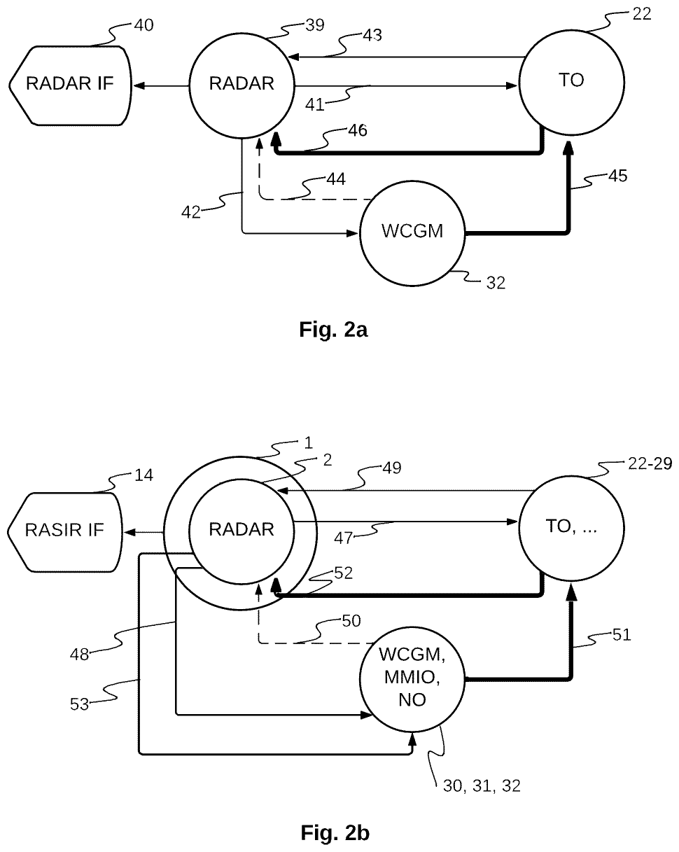

FIG. 2a shows a prior art RADAR system 39 where interference signals 46 are problematic and reduces Signal to Noise Ratio (SNR) for the RADAR system 39.

The RADAR 39 generates RADAR IF 40 data for a RADAR operator or external system. First the RADAR 39 emits a pulse 41 towards TO 22. The RADAR also emits a pulse 42 towards a Wideband Chaos Generating Material 30, MMIO 31, NO 32, disturbing the RADAR 39 by reflection 44 and by emitting interference frequency pulse signals 45 towards the target object 22. This causes the Target Object 22 to return not just its useful reflected pulse 43, but also interference reflected pulse signals 46, reducing the Signal to Noise Ratio (SNR), as error data, thus this is problematic for any RADAR data consumer 40.

### Preferred Embodiment

FIG. 2b shows a new proposed RADAR Signal Interference Recognition (RASIR) 1 using system interference signals 51 as an information resource 50, 52, to increase the bandwidth for target object frequency-based interrogation 51, and as slave signal sources 51 for improved mapping of target object locations. The RASIR system 1 interacts with both target objects 22-29 and Wideband Chaos Generating Material 30, MMIO 31, and NO 32 of all variations, and then presents an interface to access RADAR data (RASIR IF) 14 and object classification consuming interface 14, or application 14. The RASIR system's 1 usage of a metamaterial 31 as a slave RADAR signal 51 generator can be seen as a reverse ray-tracing localization of target objects having multiple chirp signatures and locations in an environment, in relation to a MIMO RADAR 2 transmitter and antenna configuration.

This constitutes a preferred embodiment of the invention comprising the features of the second embodiment of the RASIR system 2.

FIG. 2b shows that the RASIR system 1 controls its RADAR 2, and is serving an application interface RASIR IF 14.

The RASIR system 1 controls the RADAR to emit a pulse or RADAR Chirp signal 47 towards a TO 22-20, which reflects a pulse and frequency signature 49 to the RADAR for optional Time of Flight RADAR estimation. The RADAR 2 interrogates an WCGM 30-32 using a pulse/Chirp 48, the WCGM then reflects this pulse 50 back to the RADAR 2. The WCGM also emits an interrogation signal in the form of an interference signal 51 to TO 22-29, which reflects this signal back as a frequency signature 52 and pulse 52 to the RADAR 2, and RASIR System 1 for analysis. RASIR may then trigger the WCGM 30-32 to act as a slave interrogation signal 51 generator, by an aware triggering 53 of the WCGM using a pulse or RADAR Chirp. The aware triggering of WCGM 53 may be separated in time, frequency, or space from the triggering of the WCGM 30-32, as the aware triggering of WCGM to generates a controlled altered transmitter interrogation signal (ATIS) 36, 51, useful for further interrogation of the TO 22-29, and other WCGM 30, MMIO 31, as well as natural objects NO 32.

### Fourth Embodiment—RASIR with TOwNLF

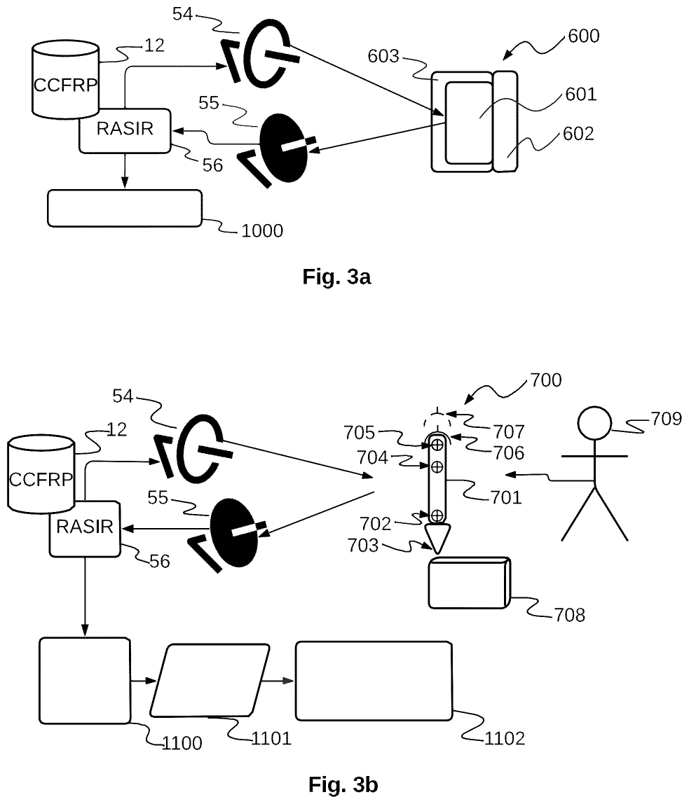

FIG. 3a shows a target object in the form of a non-linear frequency dependent RADAR signature generating target object being a RADAR tag, a Target Object with Non-Linear Frequency (TOwNLF) dependent response signature, intensity, and sampling model.

This constitutes a fourth embodiment of the invention.

According to a fourth embodiment, the RASIR system 1 incorporates as millimeter RADAR target objects 22-32, where the Target Object with Non-Linear Frequency dependent sampling model (TOwNLF) 26, also denoted as the passive RASIR Tag 600. The TOwNLF and RASIR tag (26, 600) are configured for usage in the RASIR system 1, and RASIR super system 4, and with previous mentioned embodiments. Both the TOwNLF 26 and the RASIR Tag 600 are made of material having a characteristic frequency dependent millimeter RADAR response signal signature 34, 36 sampling model. These response signal signatures 34, 36 are designed for identification and classification of target objects in the RASIR system, to be recognized in a digital signal frequency domain analysis for frequency signal response sampling model matching in the RASIR system's 1 Pattern Matching Function 10.

To increase differentiation precision for target object 22-32 in the signature sampling model pattern matching function (PMF) 10, the RASIR system may differentiate and recognize target objects based on certain target objects' unique non-linear frequency dependent signal response sampling models. For example, a sugar-water solution has a non-linear frequency dependent response pattern sampling model that follows the model of a third degree equation function, which can be uniquely recognized as a signal response pattern independent of signal strength, distance from RASIR system antennas to target object, and to some degree invariant to the target object's silhouette Radar Cross Section (RCS) area presented for the RASIR system's millimeter RADAR system 2. Such target objects are denoted TOwNLF 26 and may be further used in other target objects 22-29 as single TOwNLF and in combinations such as in the LTO 24. TOwNLF:s also exists as natural occurring target objects NO 32, Wideband Chaos Generating Material (WCGM) 30, and metamaterial MMIO 31 and target objects inside a human body TOIB (29) such as but not limited to body tissue and organs containing water, blood, and other body fluids.

Hence, according to a fourth embodiment, the RASIR Target Objects 22-32 especially the RADAR tag 600 can demonstrate properties of being Target Objects with Non-linear Frequency dependent response signature (TOwNLF) 26, comprising a substance 601 having a characteristic non-linear frequency dependent millimeter RADAR signal response signature sampling model (34, 36).

Also, according to a fourth embodiment, a RASIR tag 600 comprises a physical structure of:
a) a substance 601 having a non-linear frequency dependent radio signature, such as a carbon hydrate, or a sugar and water solution 1003 of Brix (Bx°) value 10, 20, 30, 40, 50, 60, 70, 80, or 90; and
b) an enclosure 603 protecting and preventing the sugar water solution from evaporation, escaping, or drying while being at least partially transparent for the millimeter RADAR signal spectrum in use, and
c) an optional a carrier material 602 made of a millimeter RADAR reflecting material unless the enclosure 603 is not covering the whole substance 601.

Mentioned physical structure may be based on other non-sugar-based solutions and substances in step a) demonstrating a unique TOwNLF sampling model behavior at different substance concentrations. Once each TOwNLF has been configured for a unique sampling model pattern, the object may be sent to the RASIR system 1 to be recorded and given a data record in CCFRP 12 with unique RTIS 37 and RIRS 38 pairs. Then, the TOwNLF is ready to be recognized by the RASIR system 1.

According to a fourth embodiment, the millimeter RADAR target object 22, 25, 26, 27, 28, 29, and passive RASIR tag 600 would be of the target object type, Target Object Detecting Solution (TODS) 25, which comprises,
a) a carbon hydrate such as a sugar and water solution 601 of Brix grade (Bx°) value having a known first value
   where the carbon hydrate or an absorbing material such as sodium polyacrylate, polyacrylamide (water gel), and a water solution is configured to alter its frequency dependent characteristic sampling model function when a fluid, such as water is added;
b) if water is added, then the solution would reach a second concentration value, as the solution concentration changes, which also would alter the TOwNLF characteristic signal sampling model's that is characteristic for a certain solution concentration.

The TODS 25 mentioned may then act as a remote water or fluid sensitive sensor for the RASIR system 1. Hence the TODS 25 is suitable as a remote water-fluid alarm, for but not limited to High Frequency Detection (HFD) Diapers 28, 801 receiving water 802, wound dressings receiving body fluids such as urine and blood, and for monitoring of Target Objects Inside a Body (TOIB) 29 for measuring water concentration in body tissue.

### Fifth Embodiment—RASIR with LTO

FIG. 3b shows a positioning measuring probe 700, Location Target Object (LTO) 24, for integration and interaction with a RADAR system, RASIR, capable of tracking target object based on frequency response signature target object tags. The measuring probe, open 707 and close a lid 706, shielding a target object, preferably a TOwNLF 705, to signal a position measurement event to RASIR 1. RASIR constantly tracks the orientation and location of probe 700 and calculates in 1100 the probe position 703 places on a location or object 708. Recorded locations are saved in Object position tracking system 1100 and offered to external applications such as Logistics, boundary or object tracking system 1102, via a Recorded TO position interface 1101.

This constitutes a fifth embodiment of the invention.

In the fifth embodiment, the LTO is a positioning measuring probe 700 for measuring and inputting physical object positions and locations (708) the following variants are possible depending on budget and user needs:

A basic simple passive measurement probe stick measurement that comprises
a) a first probe stick 701 end, equipped with a first target objects tag 702, and a physical probe point 703 to indicate a physical 3D position for data registration,
b) a second probe stick 701 end having a second target object tag 704, provided with at least one of the target object tags 702, 704 where Target Objects are of with Non-Linear Frequency dependent response signature (TOwNLF) type.

Mentioned basic simple passive measurement probe stick may be used for continuous data point collection and tracking.

A mechanically RADAR signaling positioning measuring probe 700 that is based on mentioned basic simple passive measurement probe stick, while adding a mean to communicate events such as measurement events to a RASIR System 1 further adding
a) a removeable lid 707 for the second target object tag 704 or
b) a third target object tag 705, to let an operator 709 signal a data point to be collected, by opening 707 and closing the lid 706.

Mentioned mechanically RADAR signaling positioning measuring probe 700 lets a user measure positions, and locations, for physical objects by pointing at the object with the stick in relation to the RASIR System's 1 RADAR antenna arrangements 17, 18, and signaling a measurement point, and to let a user define travel paths and mark locations on floor or ground for further use for navigation and decision making for another system.

Another embodiment and variant of the mechanically RADAR signaling positioning measuring probe 700, and LTO 24 is to use a similar arrangement for continuous measurement and signaling, where the:
a) open state of RADAR signal screening lid 707 and closed state of RADAR signal screening lid 706, are
b) mechanically, or electromechanically controlled, to signal a specific measurement point event to RASIR, for example to trigger a collection of a position data sample, a continuous event of positions for signaling an ego-position for a mobile apparatus or reference RADAR reflector beacon positions.

The latest variant of the positioning measuring probe 700, and LTO 24, may even use a time-based signaling scheme to transfer information to a RASIR system 1, to not just only to store new positions in CCFRP 12, but also to signal other type of events, for example to send an alarm message or to initiate a command, or service software call.

### Sixth Embodiment—RASIR Configured for HFD Diaper for a Diaper Surveillance System

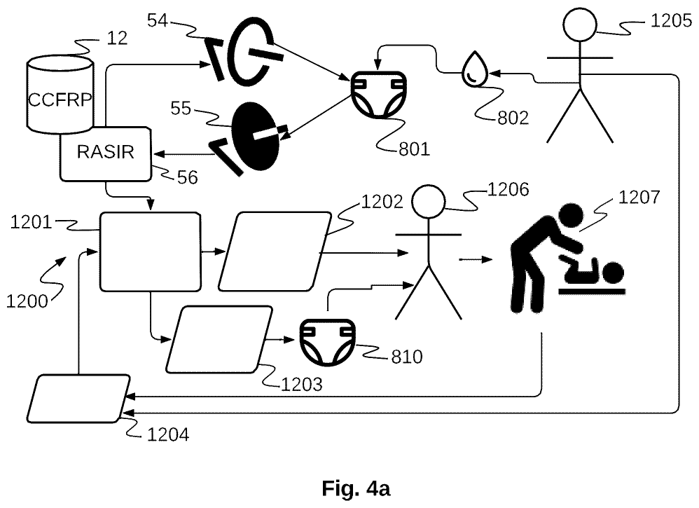

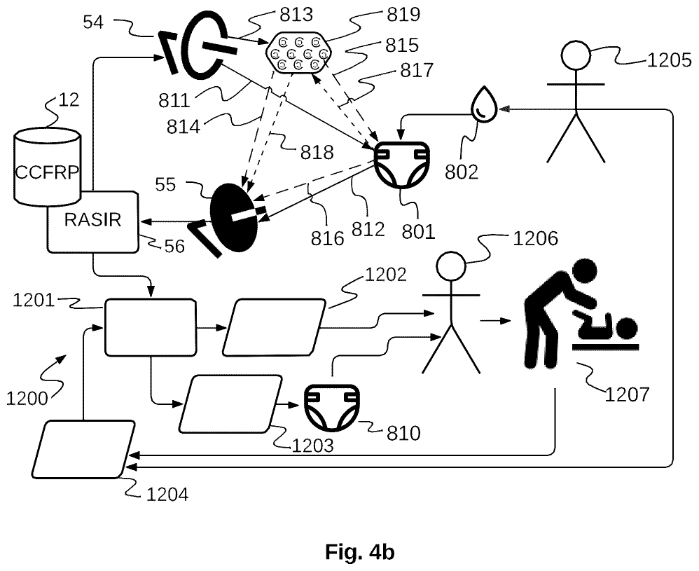

FIG. 4a shows a High Frequency Detection (HFD) Diaper 28 moisture alarm, body fluid and measurement of liquids with substances, and monitoring system 1200 measuring moisture, substance, and/or volume sensitive target objects and/or tags as at distance, based on a frequency response signature IRS and intensity that changes in relation to moisture and other substances in the diaper, substance composition, and/or volume of substance. The diaper may as well be replaced by a wound dressing serving a similar purpose. This constitutes a sixth embodiment of the invention.

In a sixth embodiment, a High Frequency Detection Diaper (HFD Diaper) 28, 801, 810 with sensor functions is remotely readable by the RASIR system 1, 56 using RASIR's RADAR subsystem 2, 54, 55, and also for applications for a HFD Diaper surveillance system 1200, where the HFD Diaper comprises a pocket to hold a fluid absorbing substance or material, where the fluid absorbing material:
a) is millimeter a water 802 sensitive RADAR target object preferably a Target Object Detecting Solution (TODS) 25.
b) demonstrates a first non-linear RADAR frequency response intensity signature when determined to be dry.
c) demonstrates a second non-linear RADAR frequency response intensity signature when a water-based fluid 802 such as a body fluid, or urine is absorbed, and the HFD Diaper 801, 810 is further designed to:
i) receive a RADAR TIS signal, or an ATIS 36 signal from a Wideband Chaos Generating Material (WCGM) 30-32, 820, 821 according to claim 33-36, and
ii) reflect an IRS signal 34, 9 to a RASIR Rx Antenna 14,
   or AIRS 34-36 via a Wideband Chaos Generating Material (WCGM) 30-32 for forwarding of the signal to the RASIR Rx Antenna 14,
   for distance surveillance of a HFD Diaper's absorbed fluid concentration, substance identification, and/or substance volume determination.

In FIG. 4a, the RASIR System 56 contains the CCFPR 12. The RADAR 54 emit an interrogation signal towards a HFD Diaper 801, worn by a care receiver 1205 who may produce water 802 that influences the HFD Diaper's 801 non-linear frequency dependent RADAR signature for a certain degree of water concentration in HFD Diaper 801, which reflects to receiving RADAR 55. The signal RADAR signal is further analyzed by the RASIR system 1 by looking up similar RADAR signature sampling models in the catalogue CCFRP 12. RASIR then forwards this information via its interface RASIR IF 14 to a Diaper care surveillance system 1201, which recognizes the identity of the HFD Diaper 1201, if earlier recorded 810 as a new HFD Diaper 810, by the Diaper ID marker 1203 operated by a Care Provider 1206. Once the HFD Diaper reaches a certain liquid concentration 802 or a certain composition of water or a solution, and for example salts, sugar, and blood components within a liquid, the Warning Graphical User Interface, and Alarm system 1202, informs care provider 1206. RASIR can also recognize the percentage of different types of salt in a liquid solution, such as urine. Once a HFD Diaper is shifted 807, the care provider 1207 and the care receiver 1205 may use a Follow Up system 1204 to generate feedback to the Diaper care surveillance system 1201.

### Seventh Embodiment—RASIR Configured for HFD Diaper Surveillance System Using WCGM

FIG. 4b shows a second diaper moisture alarm, and surveillance of compositions of a different liquid solution comprising for example salts, sugar, and blood components. The figure further shows a monitoring system configuration, for measuring moisture and/or liquid composition sensitive target object tags at distance. Where the sensor effect is based on a frequency response signature IRS that changes in relation to moisture and other liquid contained substances in the diaper. In this second diaper monitoring system configuration, the RADAR control and analysis system (RASIR) makes use of a Wideband Chaos Generating Material (WCGM) 819 to increase RADAR bandwidth and ability to penetrate material at longer wavelengths, to improve precision and ability to reach thru material when interrogating the diaper material and its moisture binding material, such as sodium polyacrylate (SPS), also known as water gel powder, or acrylamide having similar properties.

In a seventh embodiment the invention is a RASIR system interface (RASIR IF) 14 based HFD Diaper Surveillance System 1200 for monitoring and sensing of changes in signal signature sampling models for RADAR frequency response patterns from HFD Diapers 801, 810, including:
a) a HFD Diaper 801, 810 based on a TODS 25 target object,
b) a RASIR system 1 with CCFRP 12, to let CCFRP store RTIS and RIRS pairs to describe HDF Diaper 801, 810 RADAR signatures for at least 2, 3, 5, 8, or 10 different fluid concentrations, and identified types fluid, if known
c) a diaper care surveillance system 804 implemented a software program executing in a computer, or implemented in analogue and digital electronics, or as a cloud software service
d) a warning GUI alarm system 807, arranged to alert care providers 806 if a HFD Diaper 801, 810 needs to be shifted,
e) and the system may be equipped with an optional Diaper ID marker system 809, that lets care provider register new HFD Diapers 810, or to let manufacturers of HFD Diapers pre-register new HFD Diapers in a RASIR system's CCFRP database,
f) and a Follow up system 808, configured to receive messages from the care provider 806, and care receiver (803), thus letting the care receiver influence HFD Diaper care related services and calibration parameters.

In the seventh embodiment, the HFD Diaper system may be extended and modified to become a millimeter RADAR readable Wound dressing system consisting of at least:
a) a HFD Diaper 801, 810 that is physically re-configured for usage for wound dressing applications to monitor fluid levels in a wound dressings at a distance using millimeter RADAR more specifically based on the RASIR system
b) a RASIR system 14 based HFD Diaper Surveillance System 1200 re-configured to monitor wound dressings.

Mentioned wound dressing surveillance system may also monitor HFD Diapers as the technical challenges are very similar, and use cases are very similar for HFD Diapers and wound dressing monitoring.

FIG. 4b is identical to FIG. 4a but introduces the Wideband Chaos Generating Material (WCGM) 819, which enables better precision in recognition of sensor data from the HFD Diaper 801, 810.

When the RASIR system is using WCGM 819, for interrogation of HFD Diaper 801, the WCGM 819 generates an Altered Transmitter Interrogation Signal (ATIS) 815, at a wavelength longer than the millimeter RADAR band 815, that may penetrate materials such as clothes, cotton, and other objects that otherwise may block the AITS 815 signal strength.

First the RADAR 54 emits an interrogation signal 811 towards the HFD Diaper 801 under influence of water 802, which influences HFD Diaper's RADAR signature 812, which reflect to the RADAR receiver and Rx antenna 55. Simultaneously, the RADAR was also emitting an interrogation signal 813 towards WCGM 819, in this case in the form of a MMIO meta material 31 capable of transforming its received RADAR signals into new RADAR signals of a wider range of frequency spectrum, especially having penetrating wavelengths longer than 1 millimeter Altered Transmitter Interrogation Signal (ATIS) 36, 815. The WCGM reflects a signal back to RADAR Rx antenna 55, and the HFD Diaper 801 may reflect the ATIS 815, as a new IRS 816 to the RADAR Rx Antenna 55. The HFD Diaper 801 may also return the ATIS 815 back to the WCGM 819 as an AIRS 817, which is then reflected as an IRS (818) from the WCGM 819 to the RADAR Rx Antenna 55. The RASIR System 56 with then process the signals using its database CCFRP 12, to extract RASIR TLC 13 data for use via a RASIR IF 14 interface, or an application such as the Diaper Care surveillance system 1200.

### Eight Embodiment—RASIR as System for Non-Invasive Inspection of Body Using WCGM

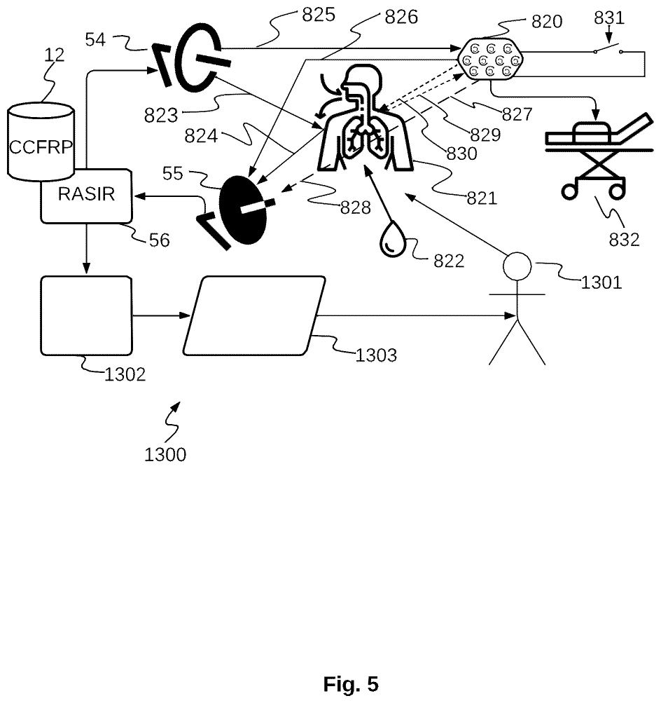

FIG. 5 shows a millimeter RASIR, RADAR system for non-invasive inspection of a human or animal body using Wideband Chaos Generating Material (WCGM), such as metamaterial to enhance frequency spectrum to reach into and thru a body to typically measure solution-based concentration in organs, intestines, lungs, and blood vessels. Also, volumes and substance composition can be identified using this method.

Different solutions such as water with a salt, and or sugar concentration of a known percentage, have a for RASIR recognizable characteristic non-linear frequency dependent signal response pattern. Hence, RASIR can recognize, measure, and determine not just amount of fluid concentration, but also more detailed information about the tissue being studied and measured, such as its concentration of salt, sugar, sucrose, blood, and acid, as a non-invasive measurement method. This constitutes an eight embodiment and an aspect of the invention.

FIG. 5 also shows a RASIR System 56, 1 with a catalogue CCFRP 12, where the RASIR System's RADAR Tx antenna 54, transmits a TIS 823 towards a human body being inspected 821. The body contains organs having different characteristic salt, sugar, acid, glucose, blood, ingested, digested and sometimes even foreign substances such as medicine and non-wanted objects; all being measurable as concentrations of substances in a solution-based 822 volume inside the human body. When RASIS performs a non-invasive analysis of the human body, the TIS is reflected from the body 821 to a Rx RADAR antenna 55 for further analysis and sampling model pattern matching by the RASIR System 56 using its catalogue CCFRP 12. To penetrate thru the body 821 to do non-invasive measurements of internal organs etc., of the body 821, a new typical metamaterial 31 based Wideband Chaos Generating Material (WCGM) 30-32 is introduced as 820, optionally having a switch that may further alter the signal signature 825 to 826 of the WCGM's 820. The WCGM 820 may be placed in a stretcher or medical care unit 832. Then as RADAR 54 emits a TIS 825 towards the WCGM 820, the WCGM reflects the signal as an IRS 826 from the WCGM towards the Rx antenna 55. As the WCGM 820 also transforms the received TIS 825 into a frequency spectrum having longer components with wavelength longer than 1 mm, the WCGM 820, also transmits its generated ATIS 827 thru the body 821 acting as a TOIB 29, where the body's 821 scanned TOIB volumes alter the ATIS 827 into a new IRS signal 828 that provides information about the human body 821 such as its solution-based concentration of substances as mentioned, that are for example: salt, sugar, glucose, blood, acid, and foreign substances such as medicine and foreign objects) as recognizable as a result of the TOIB 29 RADAR signature sampling model 827-828. Once the TIS 828 reaches the RADAR Rx antenna 55, then the RASIR System 56, 2 may perform further signal analysis.

The WCGM 820 reflected ATIS 830 may also reflect 829 on and into the body 821 tissue and back as an AIRS to the WCGM 820, and further back as an IRS (826 from the WCGM carrying signal information from the reflection 830-829 with the body 821. Then the RASIR system extracts information about internal body organs. Preferably the RADAR antennas 54, 55 would be of MIMO or SAR MIMO type to be able to scan body tissue volumes as TOIB 29 with good resolution.

The RASIR system 1 then forwards the information RASIR TLC 13 to Medical Care System 1300, for no-invasive patient inspection system using Wideband Chaos Generating Material (WCGM) 30, MMIO 31, and NO 32 to improve the RADAR system's ability send millimeter RADAR waves into the human body to collect information from reflections from lower frequency waves generated by the Wideband Chaos Generating Material (WCGM) 30 to 32.

Then, a medical care Information processing system 1302 uses RASIR TLC 13 data such as: tracking data, doppler shift of RADAR signals, and RADAR signal paths from relative different geometrical locations, to produce information about, internal body fluid composition, flow in blood vessels, fluids in lungs, bladder, bowels, brain, and to monitor heart beats.

The medical care information processing system may trigger altered configurations, and use of Wideband Chaos Generating Material (WCGM), as well as modifying the Wideband Chaos Generating Material's impedance circuit by a switch 831.

Generated information is stored and presented as medical care information 1303 for a medical care provider 1301.

### Nineth Embodiment—RASIR as a Method for Sampling Model Matching of TIS and IRS in Multiple Dimensions

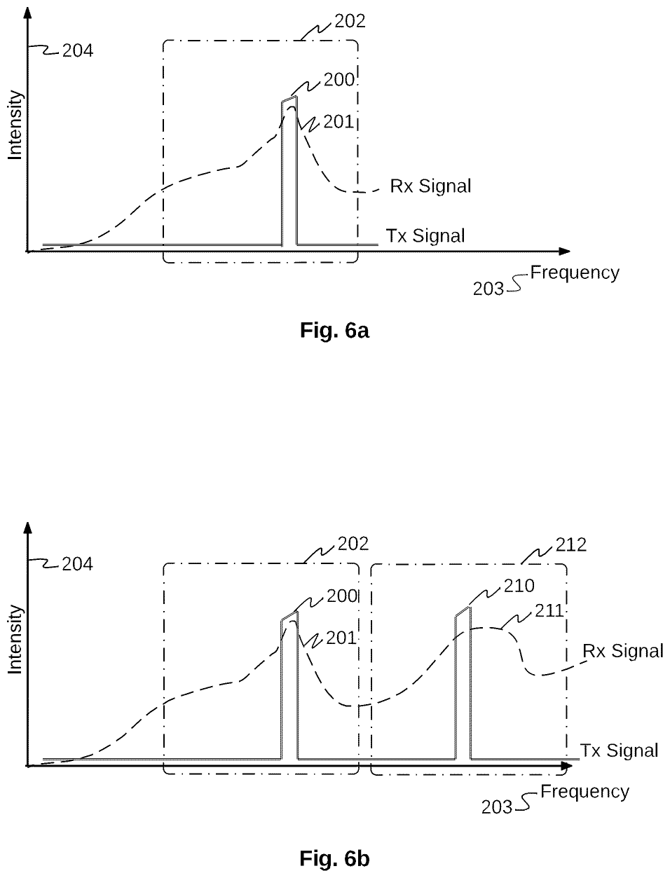

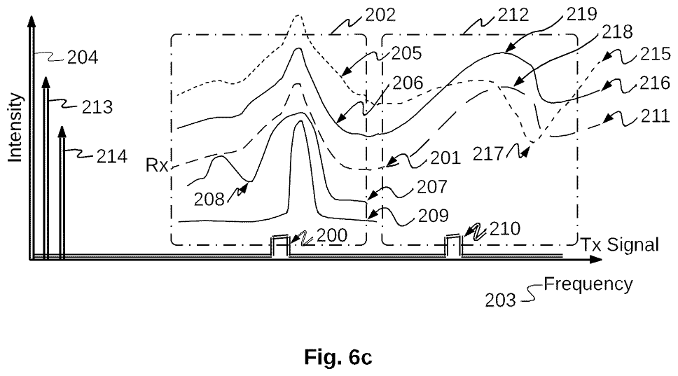

FIG. 6a-c shows sampling model matching of transmitter interrogation signal (TIS) and interrogation receiver signature (IRS) in multiple dimensions, where pairs of transmitted and received signals are analyzed and presented as a signature sampling model, and then matched towards previously stored reference transmitter interrogation signal (RTIS) and reference interrogation receiver signature (RIRS), stored in the Catalogue of Characteristic Frequency Response Patterns (CCFRP) database function, or equivalent mechanism.

FIG. 6a shows a transmitted TIS 200 and received IRS 201 in a matching window 202, where the signals have been transformed thru an FFT and presented in a frequency 203-intensity 204-plane for further sampling model matching of signature sampling model, independent of distance between target object and RADAR antennas.

FIG. 6b shows two matching windows 202 and 212 after having interrogated the target object 22-32 using two different TIS, a first TIS 200, and a second TIS 211, resulting in a first IRS 201, and a second IRS 211.

FIG. 6c shows a in matching window 202, the matching of the pairs TIS 200, 210 and IRS 201, 211 with response stored in CCFRP 12 as RTIS 37 and RIRS 38 with RIRS candidates 205, 206, 207, 209, and RIRS candidates 215, 216, 219 for the second window 212, describing and comprising a signature sampling model of target object candidates sharing or having different frequency response characteristics for a given TIS.

The sampling model pattern matching may be made in a frequency 203-intensity domain 204, or by pattern matching for matching doppler shift 213, or for polarization 214, also pattern matching in a time domain is also possible, as for any traditional RADAR system.

Previous description and FIG. 6a, 6b, 6c constitutes a nineth embodiment of the invention, which is being used by all other embodiments mentioned.

### Tenth Embodiment—RASIR Method for Basic TO Recognition

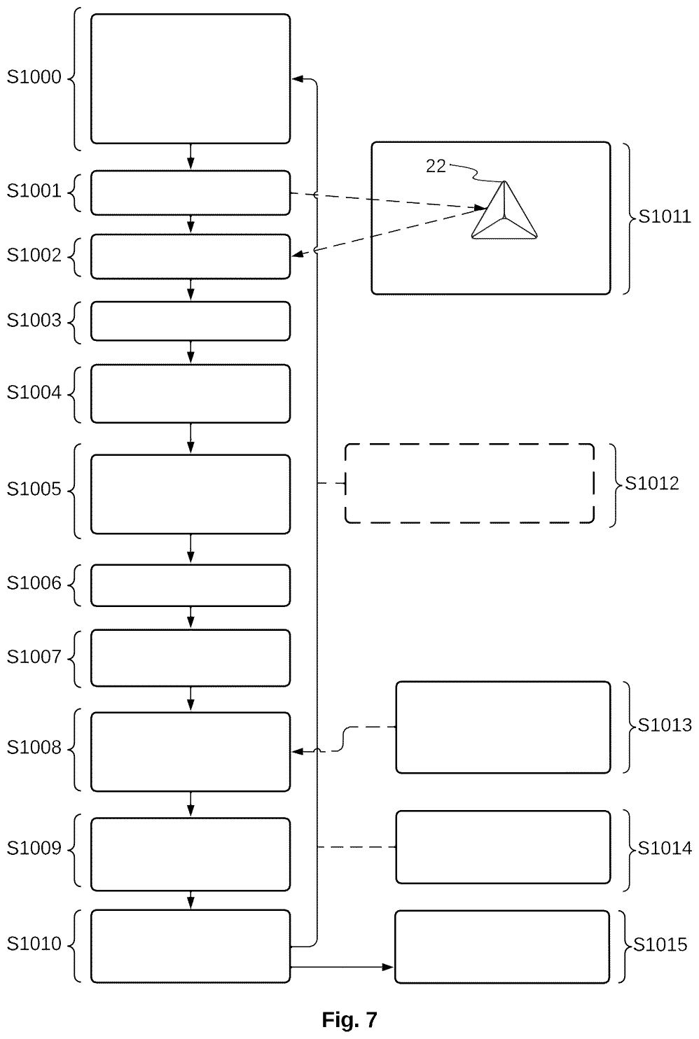

FIG. 7 shows a Method for basic recognition of Target Objects 22.

FIG. 7 describes the following steps for a basic direct TO recognition situation without any Wideband Chaos Generating Material such as WCGM, MMIO, and NO. Method steps are as follows:
S1000: Generate Tx Signal, Transmitter Interrogation Signal (TIS) with known frequency pattern:
   a) narrow frequency signal,
   b) Chirp with ramping, falling or triangular frequency sweep,
   c) Chirp with exponential frequency sweep, or
   d) frequency hopping signal (FHS)
S1001: RADAR transmits TIS Tx signals, typically 100 times.
S1011: TO reflects TIS 7 as IRS 9 back to RADAR Rx antenna 18.
S1002: RADAR receives Rx signals.
S1003: Tx and Rx are filtered and mixed.
S1004: RADAR identifies a first Target Object TO based on TOF or FMCW RADAR principles.
S1005: TO angle of arrival data is used for phase shifted data integration corresponding to a signal source from the TO location, relative the Tx and Rx antenna configuration location.
S1006: FFT analysis of received TO angel of arrival data vector.
S1007: Generate an IRS Interrogation Response Signature for the Target Object to be analyzed.
S1008: Make pattern matching of IRS to identify *matching reference RIRS identity stored in the Catalogue of Characteristic Frequency Response Signatures (CCFRS).
S1009: Store improved data on TO object classification precision based on related TIS signal used as Tx signal.
S1010: If information on object classification and position is not sufficient, repeat interrogation with next TIS signal.
S1011: As described above.
S1012: Precision improvement loop, to be executed 1 to 256 times per target object. By having detected the IRS class, one may adjust the TIS signal to:
   a) discriminate non-TO signal characteristics, and
   b) to improve the calculation of direction of arrival of characteristic recognized IRS TO signals as phase and time-of-flight (TOF) differences, especially for MIMO-antenna configurations
   As a result, the system having a MIMO antenna configuration, determines which angle of arrival and TOF distance aligns with the TIS and expected IRS signal received. This lets the system iterate and interpolate or predict by signal processing algorithms such as MUSIK (a known method) to improve precision in determined angel of arrival, and distance to each target object (TO) being interrogated.
S1013: Pattern matching assumes that the TO object may have unique a non-linear frequency dependent signal response signature based on intensity, strength, signal quality, phase for doppler shift, and polarization.
S1014: REPEAT LOOP IF:
   a) insufficient QoS;
   b) interrogation matching of TO requires more frequency response patterns and TIS signals;
   c) certain matching requires more frequency response data at other Tx frequencies.
S1015: When sufficient Quality of Service (QoS) for sampling model matching is reached. Offer improved object classification and position based on best matched frequency response signal pattern via an external interface, RASIR IF 14.

This constitutes a tenth embodiment of the invention.

### Eleventh Embodiment—RASIR Method for Recognition of TO Using WCGM

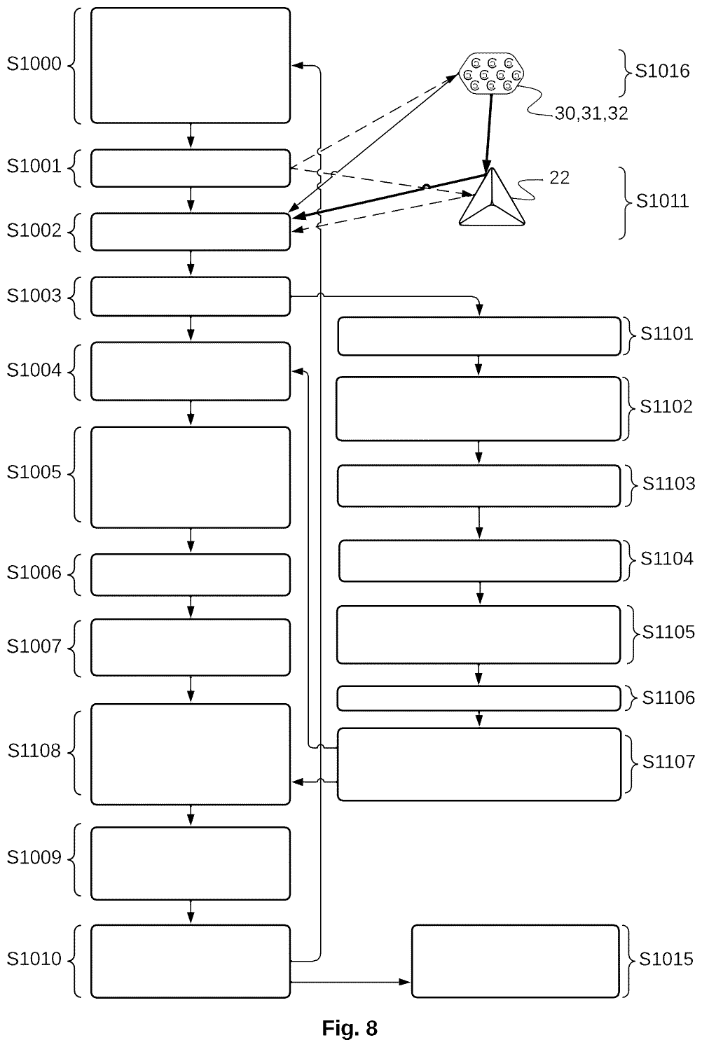

FIG. 8 shows a Method for recognition of TO using WCGM.

FIG. 8 methods steps are described as deviations and added steps based on previous methods step description. Steps not described are identical with previous description. Steps are as follows:
S1001: This step is as described earlier but in this situation an Wideband Chaos Generating Material (WCGM) 30, or Metamaterial Interference Object (MMIO) 31, or Natural Object (NO) 32; is provided as a receiver of signals.
S1006: Wideband Chaos Generating Material (WCGM) relaying interference signal at new frequency.
   The WCGM 30-32 also generates ATIS signals transmitted to the Target Object being interrogated (TO) 22-29,30-32. Signal paths are described as arrows.
S1011: The Target Object may generate two signals, one direct IRS based on first TIS from the Radar Tx; and then a second IRS based on the ATIS 36 from the Wideband Chaos Generating Material (WCGM) 30 or metamaterial 31.
S1101: RADAR identifies a first Wideband Chaos Generating Material (WCGM) based on TOF or FMCW RADAR principles.
S1102: WCGM angle of arrival data is used for phase shifted data integration corresponding to a signal source from the WCGM location, relative the Tx and Rx antenna configuration location.
S1103: FFT analysis of received WCGM angel of arrival data vector.
S1104: Generate an IRS Interrogation Response Signature for the WCGM to be analyzed.
S1105: Analyze Frequency response pattern at TIS frequency, to map frequency response for the WCGM, and save as WCGM frequency response pattern.
S1106: RADAR transmits TIS directed towards WCGM.
S1107: Wideband Chaos Generating Material (WCGM) 30, re-transmits signal at given alternative frequency, towards Target Object (TO). WCGM acts as a slave wideband transmitter: WCGM_TIS.
S1108: Make pattern matching of IRS to identify the best matching signal pattern identity stored as a matching RIRS 38, using the Catalogue of Characteristic Frequency Response Patterns (CCFRP) 12 correlated with the TIS 7 plus generated WCGM_TIS. TIS can also be matched with ATIS.

This constitutes an eleventh embodiment of the invention.

### Twelfth Embodiment—RASIR Method for Recognition of STO Using WCGM

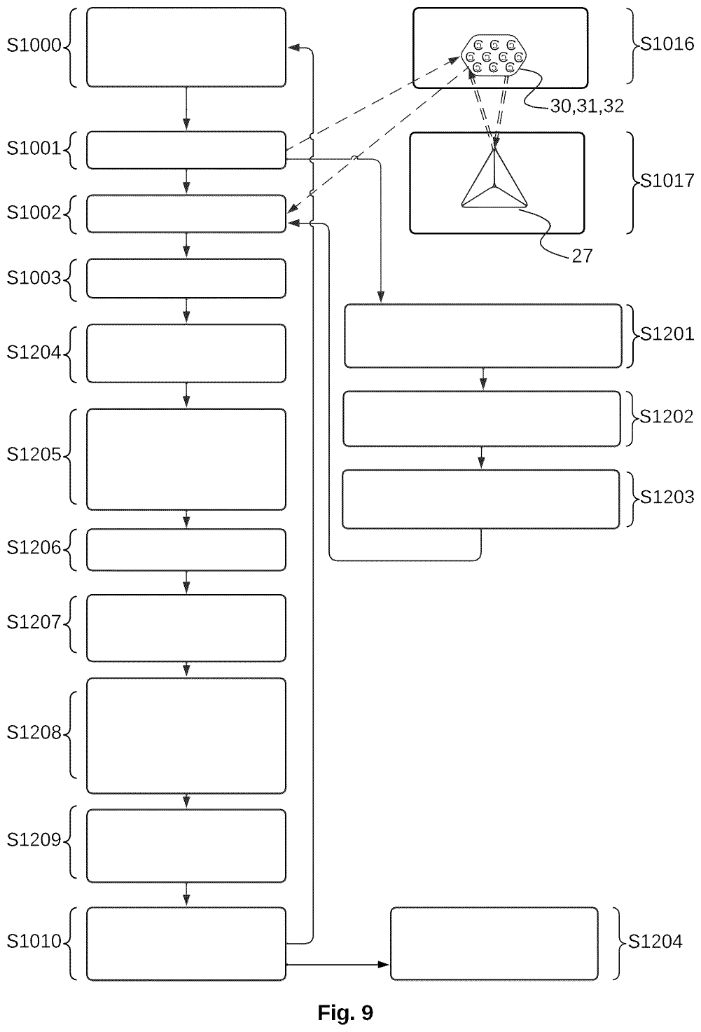

FIG. 9 shows a Method for recognition of STO using WCGM.

Only added or altered steps and items are described for the method allowing the RASIR system to interrogate objects that cannot be reached directly, called Shielded Target Objects (STO) 27. An STO may be interrogated using a Wideband Chaos Generating Material (WCGM) 30, MMIO 31, and TO 32, preferably a metamaterial configured to transform its received signals from one frequency spectrum within the millimeter RADAR bands, into a reflected signal ATIS 36 having frequency components of longer wavelengths able to penetrate the Shielded compartment 27 wherein the STO 27 is located.

Although the STO may reflect its signal back to the RADAR Rx Antenna 18, it may also and preferably in this situation relay the signal back to the Rx Antenna 18 via the WCGM 30-32. If the WCGM is designed as a metamaterial (MMIO) 31 consisting of a surface structure with resonance circuits having a frequency transformation function due to its resonance, then such an MMIO 31 could preferably transform longer wavelengths back into millimeter RADAR wavelengths.

The method in FIG. 9 is described as follows:
S1016: Wideband Chaos Generating Material (WCGM) is relaying interference signal at new frequency, thus translating a TIS 7, 33 into an ATIS 36 of new frequency. After reflection with the STO 27, the WCGM 30-32 may reflect the received AIRS 335 from the STO 27 into an IRS 34, 9.
S1017: Shielded Target Object (STO) 27 being interrogated via the WCGM 30-32.
S1201: WCGM, re-transmits received TIS 7, 33 signal as new signal ATIS 36 at given alternative frequency having a longer wavelength than 1 millimeter, towards Shielded Target Object (STO). WCGM acts as a slave wideband transmitter WCGM_TIS.
S1202: STO 27, reflects and returns a signal AIRS 35 having a frequency response based on its own characteristic and signal signature sampling model.
   RADAR reflection signature; back towards the Wideband Chaos Generating Material 30, and MMIO 31, and NO 32.
S1203: Wideband Chaos Generating Material (WCGM) 30, MMIO 31, TO 32 receives a RADAR signal AIRS 35 from the STO 27, and transforms the signal into another signal IRS 34, 9, which is relayed back to the Rx Antenna 18.
S1204: RADAR identifies a WCGM 30-32 based on TOF, FMCW, a combination, or similar RADAR principles. Once identified by the PMF 10, then the WCGM's signal signature sampling models, defined by its RTIS and RIRS pairs, is saved in catalogue CCFRP 12.
S1205: WCGM angle of arrival data is used for phase shifted data integration corresponding to a signal source from the WCGM location, relative the Tx and Rx antenna configuration location.
S1206: FFT transformation of received WCGM angle of arrival data vector.
S1207: Generate an IRS Interrogation Response Signature for the Wideband Chaos Generating Material (WCGM) 30 under influence of STO, to be analyzed.
S1208: Make pattern matching of IRS to identify the best matching signal *pattern identity, using the Catalogue of Characteristic Frequency Response Patterns (CCFRP) 12, and analyze variations due to the influence of the STO.
S1209: Store improved data on WCGM and related STO object classification precision based on related TIS signal used as Tx signal.
S1210: Offer improved object classification and position based on best matched frequency response signal pattern, covering WCGM and STO data.

This constitutes a twelfth embodiment of the invention.

In a twelfth embodiment the RASIR System 1 is configured as integrated with a Logistics Surveillance and Tracking System, typically for tracking of contents inside boxes and containers. Millimeter RADAR signals cannot reach thru walls of thick box material, nor thru non-metallic container walls. RASIR can interrogate target objects TO-NO 22-32, when the TO-NO 22-32 are located inside a logistics box, if the box's walls permit the RASIS RADAR signals thru its walls, or via an opening of the box's walls.

This twelfth embodiment describes a system that uses Wideband Chaos Generating Material (WCGM) 30 or specially configured metamaterials (MMIO) 31, to transform and transfer millimeter RADAR waves thru a wall of a logistics box, or thru an opening in the wall. If the WCGM is integrated or proximate to the box, it may increase the RADAR wave's ability to penetrate the box walls, while increasing the bandwidth of the signals that reaches into the box and reflects back to the RASIR receiver.

With boxes made of material that can be penetrated using mm RADAR, the RASIR system may interrogate such a box to investigate material contained. RASIR then analyzes reflected signals for frequency dependent non-linear patterns reflected or originating from objects and substances having known patterns recognizable by RASIR's PMF and pre-registered in CCFRS.

The WCGM and MMIO then acts as a lens or mirror for the RADAR system, which lets the millimeter RADAR 2 interrogate a container contents for shield target objects 27 via a Wideband Chaos Generating Material (WCGM) 30 or metamaterial 31, wherein: the Wideband Chaos Generating Material (WCGM) 30 or metamaterial 31, receives TIS messages from the RASIR System 1 and emits ATIS 36 towards interior of the container, to further interrogate target objects 22-32, which emits AIRS 35 towards a Wideband Chaos Generating Material (WCGM) 30 or metamaterial 31, which emits a signal 34 pulse, Chirp or designed waveform response reflected or emitted from at least one target object including WCGM and MMIO to the Rx Antenna 18 and RADAR 2, to the RASIR subsystems 19, 20, 21, 8 where the IRS 9 data record formed in RASIR.

### Thirteenth Embodiment—RASIR Learning Function for Mapping and Categorization of New Target

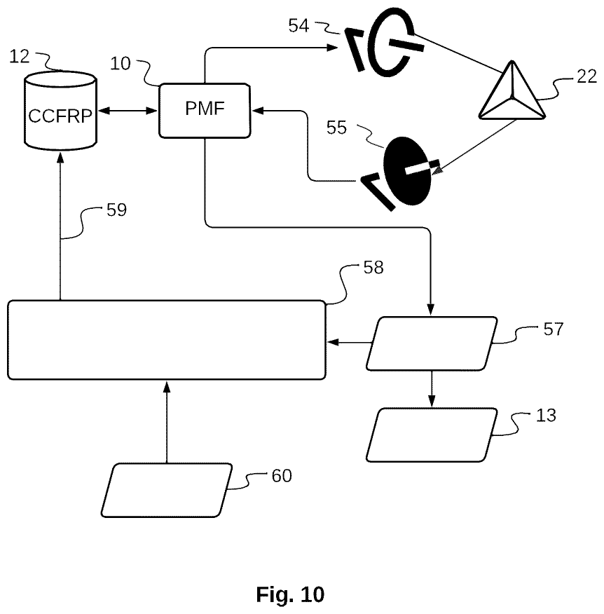

FIG. 10 shows a Learning function for mapping and categorization of new target objects and tags based on frequency response IRS detected in a scene visible for the RADAR.

This constitutes a thirteenth embodiment of the invention.

FIG. 10 describes the learning method as follows: The RASIR system 1 transmits a RADAR Tx 54, 17 signal TIS 7, 33 towards a TO 22 (22-32), which returns an IRS 34, 9 signal to the RADAR Rx 55, 18.

The Pattern Matching Function (PMF) 10 tires to match the TIS 7 and IRS 9 sampling model pairs towards any existing RTIS 37 and TIRS 38 pairs in CCFRP 12.

In the situation described, PMF 10 cannot find any matching pair in CCFRP 12.

The RASIR System 1 then transfers all pattern matching and analysis data, location, distance, RCS, and other aggregated data such as a sugar solution concentration, temperature, etc. for ATO 23, LTO 24, TODS 25, HFD Diaper 28, and TOBI 29. This data is placed in a datagram RASIR RLC 13, to describe the identified TO class 57 or type of object. For example, if no perfect match of signal signature sampling models can be made, RASIR may still classify the type of Target Object (TO) 57 based on the RASIR System's 1 interrogation and analysis results in PMF 10, and CCFRP 12.

When the RASIR System 1 is set in a Learning (or training) mode 60, it may activate a function to learn the new Target Object found: Programming of new tags 58.

Programming of new tags 58 then
asserts that the target object TO 57, 22-32, can stored in CCFRP 12.

It then request to save the target object's signal signature sampling model and TIS and IRS pairs as new RTIS and RIRS in a new data record in CCFRP 12 for future recognition of same and similar target objects 22-32.

### OTHER EMBODIMENTS

In the following section, a detailed description the apparatus and its functions with alternative and combinable embodiments are described and explained.

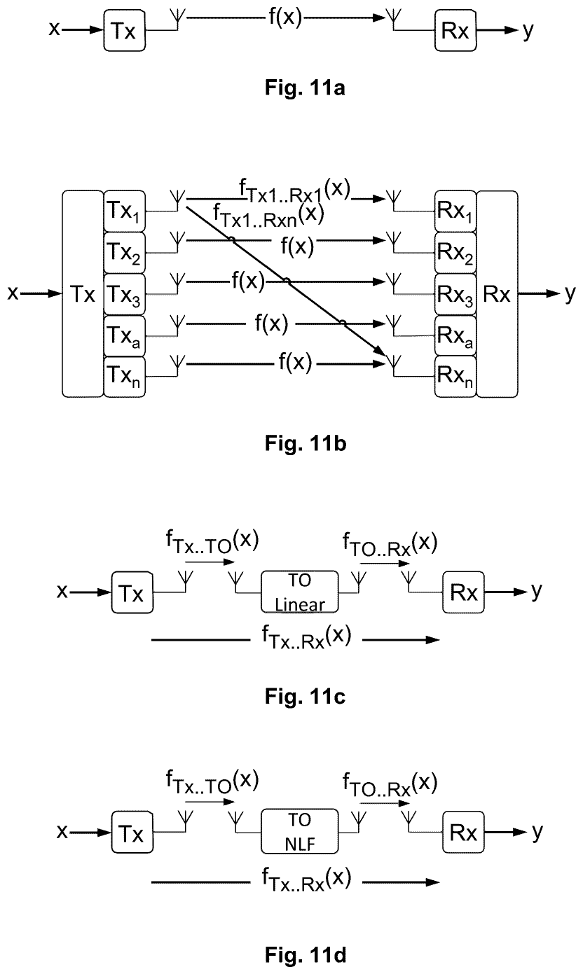

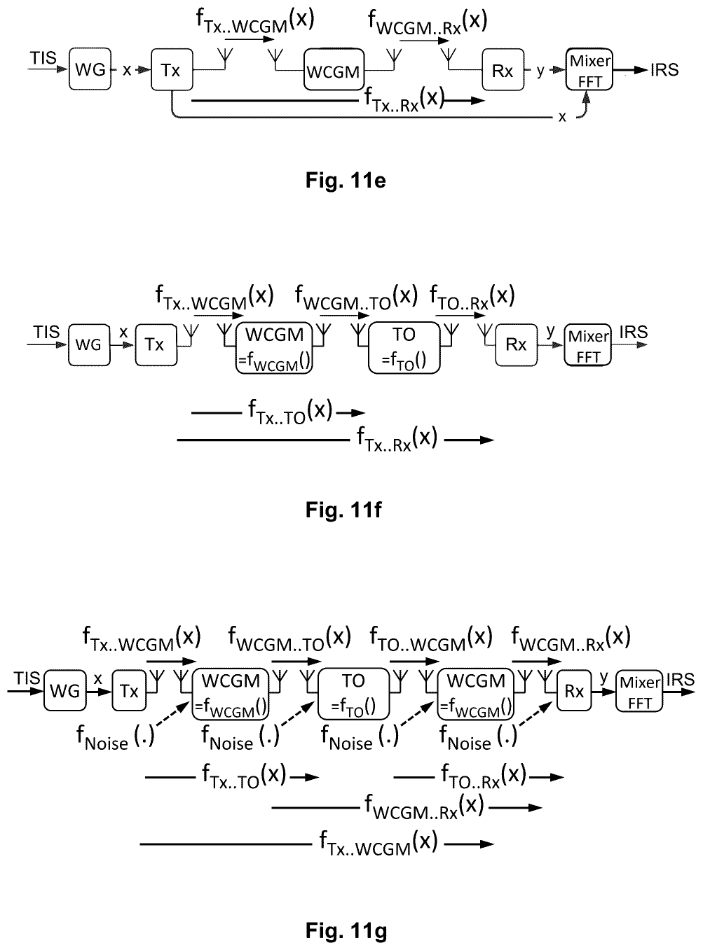

FIG. 11, shows different RADAR communication scenarios that are applicable for the RASIR system.

In one embodiment, the millimeter RADAR reflection material in at least one of the target objects, at least a TOwNLF comprises a water-sugar solution having a specified recorded Brix ° Bx value, resulting in a Brix value unique, non-linear frequency dependent RADAR reflection curvatures when processed as an IRS.

In one embodiment, the millimeter RADAR reflection material comprises a nonlinear frequency dependent RADAR signature dependent water-based solution comprising a mixture of at water plus at least two of the following components of sugar, glycose, salt, starch.

In one embodiment, the millimeter RADAR reflection material comprises a substance having at first state represented by characteristic nonlinear frequency dependent RADAR signature, and a second characteristic nonlinear frequency dependent RADAR signature, where the second state is activated when the reflection material absorbs a water-based fluid such as, water, sugar solution, or a body-fluid such as urine, blood, blood plasma, tears, milk, stomach contents, puke, pus, and feces.

In one embodiment, several millimeter RADAR reflection material surfaces are arranged according to a predefined pattern system, such as along a straight line, in a matrix, or in a geometric pattern defined by known angles in a 2D plane, where RADAR system may recognize reflection surface substance RADAR signature properties at a distance, and where presence of a refection surface of a certain RADAR signature in the pattern location signals a value or identity to the RADAR analysis system.

In one embodiment, several millimeter RADAR reflection material surfaces are arranged according to a predefined pattern system, such as along a straight line, in a matrix, or in a geometric pattern defined by known angles in a 3D plane, where RADAR system may recognize reflection surface substance RADAR signature properties at a distance, and where presence of a refection surface of a certain RADAR signature in the pattern location signals a value or identity to the RADAR analysis system.

In one embodiment, a millimeter RADAR position probe comprises at least two separate millimeter non-linear RADAR reflection material surfaces having known non-linear frequency dependent RADAR signature surfaces, located along a straight-line with a known distance to a location probe point.

In one embodiment, a millimeter RADAR position probe for signaling of its probe position and orientation, comprises: a moveable cover over one of the non-linear frequency dependent RADAR reflection materials having at least two states;
where in the cover in its first state shields the RADAR reflection material, and the cover in its second state exposes the RADAR reflection material, to signal a probe position and orientation to a millimeter RADAR system

In one embodiment, the RASIR system is equipped with a RADAR and target object or RASIR tag, in the form of: TOIB 29, ATO 23, LTO 24, TODS 25, TOwNLF 26, and/or STO 27, that are configurations that uses frequencies from 5 GHz, with wavelengths from 60 mm up to 300 GHz, with wavelength to 1.0 mm.

In one aspect, the RASIR system is equipped with a mm RADAR and target object tag configuration that operates using frequencies from 3 GHz, with wavelengths from 100 mm up to 78 GHz, with wavelength to 3.8 mm.

In a second aspect, the RASIR system is equipped with a RADAR that operates in the Ultra-Wideband (UWB) operates on frequency range from 3.1 GHz, with wavelength 97 mm, to 10.6 GHz with wavelength 28 mm. These RADAR frequencies are feasible for when the RASIR RADAR needs to penetrate the human body.

In a third aspect, the RASIR system is equipped with a RADAR that operates in frequency ranges from 30 GHz with wavelength 10 mm up to 120 GHz with wavelength 2.5 mm.

In a fourth aspect, the RASIR system is equipped with a RADAR that operates in frequency ranges 40 GHz to 70 GHz with wavelength from 7.5 to 4.3 mm

In one embodiment the RASIR System is captured as a software integrated with a hardware control system to execute and control functions of the RASIR System 1, according to as follows where the RASIR software 4 is configured for the execution a RASIR system 1 and RASIR super system 4, to when installed in a RASIR control system 5 or RASIS system 1, execute methods to run the control processes and operation according to the first, second and third embodiment.

The software then controls the execution of all processes of the RASIR System, resulting in a continuous or intermittent measurement of target objects 22-32 in a millimeter RADAR target object 22-32 environment 3 extended by the RASIR functionality.

Further the RASIS software is configured to control execution of information processing in at least one of the following computing system architectures:
a) a computer with CPU and/or MPU, memory, instruction memory, and communication system,
b) a Digital Signal Processor (DSP), Analog and digital filters, A wave generators, and cascade of wave generators,
c) a FPGA, which may host an MPU and several DSP:s,
d) ASIC for execution logic definition RASIR software instructions translated into VHDL code, e) an AI accelerator architecture,
f) a neural network processor or accelerator,
g) a graphics processing unit (GPU), or
h) Cloud computing.

### REFERENCE SIGNS LIST

Description of reference numbers used in drawings are defined as follows. First a figure reference number is specified, and then a short name of the item, arrow, flow of information or method step is presented, and in some cases also briefly described. Method steps if any are denoted with a prefix “S” added to a number such as S1001.
1=RADAR Signature Interrogation and Recognition (RASIR) system, comprising a RADAR sub-system.
2=RADAR, for example an FMCW RADAR, or TOF RADAR, with extended functions.
3=Target Objects (TO) for RADAR signal analysis. A TO can be a man-made or natural occurring objects, as well as a metamaterial, or a target object with a non-linear frequency dependent signature, as well as a RADAR tags 22-32.
4=The RASIR super system combining RASIR 1, and
   Target Object and Model of Environment 3 offering combinations of target object 22 and sub-classes 22-32.
5=Control System (CS) that executes the logic and control of the RASIR system.
6=Transmitter Interrogation Signal Generator (TISG).
7=Transmitter Interrogation Signal (TIS).
8=Fast-Fourier Transform (FFT).
9=Interrogation Response Signature (IRS).
10=Pattern Matching Function (PMF).
10′=Pattern matching of signal signature sampling model alignment.
11=Software, instructions or logic that defines how the system executes the target object recognition, detection, classification, localization, and remote sensing function.
12=Catalogue of Characteristic Frequency Response Patterns (CCFRP), storing at least pairs of RTIS and RIRS to support matching of signal characteristics of Target Objects (TO).
13=Target object Location and Characteristics (TLC).
14=RADAR information and application Interface.
15=Waveform Generator (WG).
16=Transmitter (Tx).
17=Tx Antenna.
18=Rx Antenna.
19=Receiver (Rx).
20=Tx Rx Mixer.
21=Target Object (TO), Position and Timed-Of-Flight extraction (TO POS TOF).
22=Target Object (TO). A target object may be in the form of any of the target object sub classes 23 to 32 and packaged as a target object tag such as a passive RASIR tag 600.
23=Active Target Object (ATO) able to transfer information by altering its frequency response due to a physical input such as temperature change, rotation of an object, exposing, and hiding a target object for RADAR interrogation.
24=Location Target Object (LTO), such as a measurement probe for measuring objects where target objects as hidden and made visible to the RADAR, as a mean to signal information. Robotic and other vehicles may use this technique to electronically signal a flashing target object as a reflector carrying a certain time dependent signature.
25=Target Object Detecting Solutions (TODS), such as a variable water sugar concentration.
26=Target Object with Non-Linear Frequency dependent response signature (TOwNLF).
27=Shielded Target Object (STO).
28=Diaper with moisture influence non-linear frequency dependent response signature High-resolution Fluid Detection (HFD) Diaper (HFD Diaper).
29=Target Object Inside Body (TOIB), such as water-based solutions in stomach, intestines, lungs, and blood vessels.
30=Wideband Chaos Generating Material (WCGM).
31=Metamaterial Interference Object (MMIO).
32=Natural object having metamaterial properties (NO).
33=Signal pulse, Chirp or designed waveform transmitted from Tx Antenna towards a Target Object.
34=Signal pulse, Chirp or designed waveform response reflected or emitted from at least one target object to the Rx Antenna for further analysis.
35=Target Object, emits or reflects a signal to a Wideband Chaos Generating Material (WCGM), after having received a RADAR pulse, Chirp, or waveform signal. This carries an Altered Interrogation Receiver Signature (AIRS).
36=This signal carries an Altered Transmitter Interrogation Signal (ATIS). Wideband Chaos Generating Material (WCGM), reflects or emits a frequency shifted, direction altered signal to a target object (TO), after having received a RADAR pulse, Chirp or waveform signal from a Tx Antenna.
37=Reference Transmitter Interrogation Signal (RTIS), stored in pairs with RIRS in CCFRP.
38=Reference Interrogation Response Signature (RIRS), stored in CCFRP.
39=A prior art millimeter RADAR system.
40=An Interface & Application making use of RADAR data.
41=A RADAR pulse, Chirp, or wave being sent towards a Target Object (TO).
42=A RADAR pulse, Chirp, or wave being sent towards a Wideband Chaos Generating Material (WCGM).
43=A RADAR pulse, or wave reflected from a Target Object (TO) to a RADAR system.
44=A RADAR pulse, or wave reflected from a Wideband Chaos Generating Material (WCGM) to a RADAR system.
45=An interference frequency pulse or radio wave transferred from a Wideband Chaos Generating Material (WCGM) towards a Target Object (TO), with or without a transformation of original RADAR pulse, or wave frequency, time alignment, strength, polarization, and phase.
46=Interference reflected pulse, a signal contribution reflected from a target object, as a result of an earlier interference frequency pulse from a Wideband Chaos Generating Material (WCGM) 45.
47=A RADAR pulse, Chirp, or wave being sent towards a Target Object (TO), or any sub-class of Target Object 22-29.
48=A RADAR pulse, Chirp, or wave being sent towards a Wideband Chaos Generating Material (WCGM), or any sub-class of a Wideband Chaos Generating Material (WCGM) 30-32.
49=A RADAR pulse, or wave reflected from any type of Target Object (TO) to a RADAR system.
50=Reflected pules & frequency signature. A RADAR pulse, or wave reflected from a Wideband Chaos Generating Material (WCGM) to a RADAR system for further analysis of the Wideband Chaos Generating Material's RADAR signature characteristics, for potential later use as slave signal generator.
51=Interference frequency pulse, reused as: slave remote RADAR signal source, enabled by WCGM awareness. An interference frequency pulse or radio wave transferred from a Wideband Chaos Generating Material towards a Target Object (TO), with or without a transformation of original RADAR pulse, or wave frequency, time alignment, strength, polarization, and phase.
52=Interference reflection pulse and frequency signature; reflected from a target object, as a result of an earlier interference frequency pulse from a Wideband Chaos Generating Material (WCGM) 51.
53=Aware triggering of Wideband Chaos Generating Material (WCGM) 30 to act as an extra remote RADAR signal resource. The RADAR may transmit a selected Chirp, pulse, narrow band frequency spike, to make the Wideband Chaos Generating Material (WCGM) 30 emit a distinct known secondary slave signal towards the Target Object (TO), for further analysis of from the TO reflected signals, by the Pattern Matching Function (PMF) 10.
54=RADAR Transmitter with antenna.
55=RADAR Receiver with antenna.
56=A subsystem with RASIR functions interacting with CCFRP 12 database function, and the interface (IF) 14, and external applications collaborating or making use of RASIR functions.
57=Identified TO Class.
58=Programmer for new tags and Target Objects (TO).
59=Update signal sent to CCFRPP with new tag characteristics containing new pair of RTIS and RIRS data records with optional location data and target object classification data and generate a new context unique identifier for the data record in the CCFRP database.
60=Learning mode, a datagram ON/OFF controlling if new Target Objects 22-32, 58, is to be recorded in the CCFRS as the PMF recognized these new target objects. If the learning mode is set to ON, the target object is recorded. RASIR may record any type of target object (TO) 22, such as: ATO 23, LTO 24, TODS 25, TOwNLF 26, STO 27, HFD Diaper 28, TOIB 29, and wideband chaos generating material objects such as WCGM 30, MMIO 31, and NO 32. The basic function of Learning mode 60, is to enable or disable learning new identity tags in the form of any type of target objects 22-32.
200=A first RADAR Transmitter (Tx) signal waveform based on the Transmitter Interrogation Signal (TIS) 7 sent towards and reflected by a target object, resulting in a RADAR Receiver signal 201.
   This Transmitter signal 200 is presented after filtering, amplification, and processing by the Fast Fourier Transform (FFT) 8, in a frequency domain chart with intensity as a vertical axis Intensity 204 on one axis and a Frequency 203 on the other horizontal axis. The Tx signal 200 may have different waveform shapes but is in the chart represented as a narrow frequency spike at a specific frequency, acting as a first interrogation signal for the target object 22-32 being investigated and analyzed.
201=A first RADAR Receiver Rx signal as captured after reflection on and in a target object 22-32, and after signal processing and FFT 8 transformation onto a frequency intensity plane.
202=A first sampling model matching scenario, and frequency window for a first analysis of multiple repetitions of Tx signals 200 and related received Rx signals 201, where Rx signals received have a much more wide frequency spectrum than the Tx signals, due to non-linear frequency characteristics of the target object, Wideband Chaos Generating Material (WCGM) 30, and metamaterial MMIO 31, and other target objects 22-32 being interrogated, due to surface, interference, cross section shape, electrical metamaterial circuits created, impedance, and resonance of dipoles in water solutions.
203=A horizontal Frequency axis.
204=A vertical Intensity axis, which may be accompanied with alternative vertical axis for analysis: Doppler shift 213, and Polarization 214.
205=An in the scenario described second best fitting, reference interrogation response signature (RIRS) 38, stored in CCFRP 12 for signal signature sampling model alignment matching with the Receiver Rx signal 201. This 2^{nd} best signal 205, 215 and received signal 201, 211 has a good alignment during sampling model matching in scenario 202, but deviates in scenario 212, compared to RIRS 206, 216.
206=As in the scenario described, best fitting 206 reference interrogation response signature (RIRS) 38. It is stored in CCFRP 12 for signal signature sampling model alignment matching in the Pattern Matching Function (PFM) 10, matching with the Receiver Rx signal 201, 211. This bet fitting RIRS 206 has another reference frequency signal signature sampling model 216 for signals based on Transceiver Tx signal 210. The matching candidates are stored as several candidate pair of RTIS and RIRS in CCFRP 12. The reference signal pairs Reference Interrogation Receiver Signature (RIRS) 216, and its Reference Transmitter Interrogation Signal (RTIS), is not shown but is substantially identical to the Transceiver Tx signal 210.
207=A bad RIRS candidate with low correlation, disqualifying matching with the received Rx signal 201, and deviations in signal signature sampling model alignment 208.
208=Signal signature sampling model deviation for a bad RIRS candidate.
209=A weak RIRS candidate with low correlation and a disqualifying matching of signal signature sampling model compared to received Rx signal 201. Such a reflected signature sampling model, or IRS is likely a result from a metallic linear frequency dependent reflecting target object material.
210=A second RADAR Transmitter (Tx) signal waveform based on a second Transmitter Interrogation Signal (TIS) 7 sent towards, and reflected by, a target object, resulting in RADAR Receiver signal 211
211=A second RADAR Receiver Rx signal as captured after reflection on and in previous target object 22-32, and after signal processing and FFT 8 transformation onto a frequency intensity plane.
212=A second sampling model matching scenario, and frequency window for a first analysis of multiple repetitions of Tx signals 210 and related received Rx signals 211, where Rx signals received have a much more wide frequency spectrum than the Tx signals, due to non-linear frequency characteristics of the target object, Wideband Chaos Generating Material (WCGM) 30, and metamaterial MMIO 31, and other target objects 22-32 being interrogated, due to surface, interference, cross section shape, electrical metamaterial circuits created, impedance, and resonance of dipoles in water solutions.
213=An alternative Intensity axis on the diagram describing Doppler shift 213.
214=An alternative Intensity axis on the diagram describing Polarization 214.
215=Deviations of second-best candidate RIRS 205 from Rx signal 211, in the second sampling model matching scenario 212.
216=Good alignment of related best matching candidate RIRS 206 and RIRS 216 during the second sampling model matching scenario 212.
217=Disqualifying signal signature sampling model deviation between the Rx Signal 211 in relation to RIRS 215 at location 217.
218=A location of a good signal signature sampling model section 218 with good alignment between the Rx Signal 211, and RIRS 216 sampling model section 219 in second sampling model matching scenario 212.
219=A location of good signal signature sampling model section 218 with good alignment between Rx Signal 211, and RIRS 216 signal signature sampling model section 219.
600=A passive RASIR tag, a Target Object with Non-Linear Frequency dependent RADAR signal response characteristics, using a carbon hydrate-water solutions, or a sugar-water-based solution of known concentration. The RASIR tag can be packaged as a physical identity tag, for a rough environment.
601=A substance having Non-Linear Frequency dependent RADAR signal response characteristics, such as a carbon hydrate-water solutions, or a sugar-water solution based solution, gel, stable non-fluid format, or cell structure.
602=A physical carrier and RADAR reflective material, carrying the substance 601.
603=A physical enclosure to protect 601, while being substantially transparent for millimeter RADAR signals.
700=A Location signaling Target Object (LTO), a position measurement probe with or without a signaling capability.
701=An outstretched LTO structure
702=A first trackable target object (TO) preferably with uniquely detectable target object with a non-linear frequency dependent response signature (TOwNLF).
703=A probe end for measurements of a 3D location.
704=A second trackable target object (TO) preferably with uniquely detectable target object with a non-linear frequency dependent response signature (TOwNLF), providing orientation information for the position measurement probe 700.
705=A third trackable target object (TO) preferably with uniquely detectable target object with a non-linear frequency dependent response signature (TOwNLF), providing orientation information for the position measurement probe 700, and signaling of presence by closing 706 and opening 707 a lid, to signal a measurement point to the position recording system.
706=Closed state of RADAR signal screening lid.
707=Open state of RADAR signal screening lid.
708=Object positions being physically measured by a Person 709. Positions are relative the Tx and Rx antenna configurations.
709=A Person, measuring object locations, borders, paths, and object proportions.
801=A diaper configured as a High Frequency Detection (HFD) Diaper with ability to signal presence of water, urine, feces, and body fluids, as a change in a frequency dependent RADAR signal response signature sampling model.
802=A measurable amount of water, urine, feces, or body fluid.
810=A new replacement HFD Diaper having a signal signature sampling model and characteristic, having a signature model that may be used for detection of water, urine contents in moisture absorbing material, inside a pocket of the HFD Diaper.
811=A RADAR signal from transmitter 54 sent to HFD Diaper 801.
812=RADAR signature signal reflected from HFD Diaper 801 to Radar receiver 55.
813=A RADAR signal from transmitter 54 sent to Wideband Chaos Generating Material (WCGM) 819.
814=RADAR signature signal reflected from Wideband Chaos Generating Material (WCGM) 819 to Radar receiver 55, likely with an altered frequency spectrum.
815=RADAR signature signal reflected from Wideband Chaos Generating Material (WCGM) 819 to HFD Diaper 801, likely with an altered frequency spectrum.
816=RADAR signature signal reflected from HFD Diaper receiver 801, likely with an altered frequency profile, to RADAR receiver 55.
817=RADAR signature signal reflected from HFD Diaper receiver 801, likely with an altered frequency profile, to Wideband Chaos Generating Material 819.
818=RADAR signature signal reflected from Wideband Chaos Generating Material (WCGM) 819 to Radar receiver 55 based on an earlier reflection form HFD Diaper 801 to Wideband Chaos Generating Material 819.
819=An Wideband Chaos Generating Material (WCGM) 30, able to reflect, and transform a received RADAR signal into a new RADAR signal with a new characteristic frequency distribution, preferably being able to penetrate the layers of a HFD Diaper having a longer wavelength than the received RADAR signal.
820=A Wideband Chaos Generating Material (WCGM) according to one of the forms 30, 32, and 32; able to translate millimeter RADAR waves to longer wave lengths with ability to reach into, or thru the human body.
821=A human body for non-invasive medical inspection using millimeter RADAR waves.
822=A body fluid, water, blood cloth, organ, feces inside the human body 821 representing a change in the concentration of water molecules in a solution.
823=RADAR signal transmitted from RADAR transmitter 54 directed towards a human body 821.
824=RADAR signal reflected from human body 821 towards a RADAR receiver 55.
825=RADAR signal transmitted from RADAR transmitter 54 directed towards a type of Wideband Chaos Generating Material (WCGM) 820, that reflects and transforms at least a part of the millimeter RADAR waves to a longer wave length having a lower frequency able to penetrate through human tissue, and body 821.
826=The RADAR signal reflected from Wideband Chaos Generating Material (WCGM) 820 towards the RADAR receiver 55.
827=The RADAR signal reflected and transformed from Wideband Chaos Generating Material (WCGM) 820 towards the human body 821, and transformed thru the human body 821.
828=The RADAR signal reflected and transformed from Wideband Chaos Generating Material (WCGM) 820, transformed thru the human body 821, where it further reaches the RADAR receiver 55.
829=The RADAR signal reflected from and transformed by Wideband Chaos Generating Material (WCGM) 820, reaches the human body 821, where at least part of the wave reflects back to the Wideband Chaos Generating Material (WCGM) 820.
830=A reflected wave 830 remaining from a wave sent from the Wideband Chaos Generating Material (WCGM) 820, reflected by the human body 821, and reflected back to the Wideband Chaos Generating Material (WCGM) 820.
831=A switch that lets the medical care provider 1303, and system 1300, to switch on and off a circuit in the Wideband Chaos Generating Material (WCGM), thus changing its frequency dependent RADAR signature characteristics. This makes it possible to measure with and without the use of Wideband Chaos Generating Material.
832=A hospital bed, stretcher, or intensive care unit system wherein a Wideband Chaos Generating Material (WCGM) may be placed to ease further investigation using millimeter RADAR based non-invasive medical examination, such as 1300. In such a bed, the switch 831 may be made accessible for medical care providers.

Applications and usage-oriented items, functions, and sub-systems follows.
1100=Object position tracking system.
1101=Recorded target object position interface, datagram.
   Recorded specific target object as passive identity tag position interface, datagram. The target object may therefor act as a passive identification tag, meaning that it does not need power to, nor any active radio transmission capability, to be used as a target object 22-32 identification tag in RASIR.
1102=Logistics, boundary or, object tracking system.
1200=A HFD Diaper, or wound dressing surveillance system.
1201=Diaper care surveillance subsystem.
1202=Warning or alarm datagram presented on a graphical user interface, voice message, electronic alarm, email or similar signal.
1203=Diaper Identity marking subsystem, for scanning and generation of a context unique identity of new diapers 810, and diaper in use 819.
1204=Follow up sub-system designed to inform and alert care providers 1206, as well as care receiver 1205, about recent and future events.
1205=A Care receiver.
1206=A Care provider.
1207=A situation where the Care provider 1206 provides care to the Care receiver 1205.
1300=A medical care system for no-invasive patient inspection system using Wideband Chaos Generating Material (WCGM) 30-32 to improve the RADAR system's ability send millimeter RADAR waves into the human body to collect information from reflections from lower frequency waves generated by the Wideband Chaos Generating Material (WCGM) 30, MMIO 31, and NO 32.
1301=Medical care provider 1301.
1302=Medical care Information processing of RASIR tracking data, doppler shift of RADAR signals, and RADAR signal paths from relative different geometrical locations, to produce information about, internal body fluid composition, dynamic flow in blood vessels, dynamic fluids in lungs, bladder, bowels, brain, and to monitor heart beats. The medical care information processing system may trigger altered configurations and use of Wideband Chaos Generating Material (WCGM), as well as modifying the Wideband Chaos Generating Material's circuit by a switch 831. Generated information is stored and presented as the medical care information 1303.
1303=Medical care information about the human body 821's internal functions, presented for a medical care provider 1301.

Numeral reference signs introduced in FIG. 7:
S1000 to S1015=Method steps to interrogate the target object 22, for target object recognition with reference objects stored in CCFRP 12.

Numeral reference signs introduced in FIG. 8:
S1000 to S1011,
and S1015=Inherited method steps from FIG. 7.
S1016=New Wideband Chaos Generating Material (WCGM) 30 introduced to represent a signal path and a method step.
S1101 to S1108=With method step S1101 to S1108 inserted to recognize the target object 22, making use of the presence of a Wideband Chaos Generating Material (WCGM) 30, a Meta-material interference material (MIMO) 31, or a Natural occurring Object (NO) 32 having similar properties.

Numeral reference signs introduced in FIG. 9:
S1000 to S1010,
and S1015=Inherited method steps from FIG. 7.
S1016=New Wideband Chaos Generating Material (WCGM) 30 introduced to represent a signal path and a method step.
S1017=New shield target object STO 27 representing a signal path and method step.
S1201 to S1204=New method steps inserted to recognize a shielded target object 27, making use of the Wideband Chaos Generating Material (WCGM) 30-32, when present.

1.-44. (canceled)

45. A method of performing remote substance analysis of a target object by a millimeter radar system, the method comprising:
A. interrogating the target object by transmitting an interrogation radar signal from a radar transmitter antenna oriented towards a space expected to comprise the target object;
B. receiving, by a receiver antenna, a reflected radar signal from the target object in dependence on the interrogation radar signal;
C. mixing the interrogation radar signal and the reflected radar signal so as to obtain a mixer signal;
D. transforming the mixer signal into the frequency domain so as to generate an interrogation response signature that expresses a signal intensity as a function of frequency;
E. pattern matching of a first pair comprising the interrogation radar signal and the interrogation response signature for the target object, with a reference pair comprising a reference interrogation radar signal and a reference interrogation response signature, wherein the reference pair is stored in a reference database; and
F. determining one or more properties of the target object in dependence on data associated with the matched reference pair stored in the reference database.

46. The method according to claim 45, further comprising, in case no matching reference pair is identified in the reference database, and the system is set in a learning mode, inserting a new data record in the reference database, wherein the new data record comprises the first pair, for later referencing and pattern matching.

47. The method according to claim 45, wherein the pattern matching comprises using a pattern matching function to find a best signal signature match of the first and second pairs using at least one of the steps:
A. matching by finding and comparing a least square error between Frequency-Amplitude signature sampling model of the interrogation radar signal and interrogation response signature pairs and reference interrogation radar signals and reference interrogation response signatures stored in the reference database;
B. matching of doppler shift sampling models;
C. matching of signals with polarization sampling models;
D. matching of frequency resonance spectrum intensity sampling models;
E. matching of frequency resonance spectrum phase sampling models;
F. matching of frequency resonance spectrum combination of intensity, phase and polarization sampling models;
G. matching of least square error in the time domain;
H. matching of non-linear frequency dependent interrogation response signatures, where the frequency response signature sampling model in an interrogation response signature reflected from the target object hit by a specific interrogation radar signal follows a third-degree polynomial equation function signature sampling model, and where extracted equation parameters are matched with corresponding parameters for previously stored reference interrogation radar signal and reference interrogation response signature pairs;
I. matching of sampling models using auto correlation between the interrogation radar signal and the reference interrogation radar signal, and between the interrogation response signature and the reference interrogation response signature;
J. matching of patterns is using cross correlation between the interrogation radar signal and the reference interrogation radar signal, and between the interrogation response signature and the reference interrogation response signature;
K. matching of patterns is using any other correlation method between the interrogation radar signal and the reference interrogation radar signal, and between the interrogation response signature and the reference interrogation response signature;
L. matching of interrogation radar signal with reference interrogation radar signal, and interrogation response signature with reference interrogation response signature using a Digital Signal Processor (DSP), or a Field Gate Programmable Array (FPGA) programmed for matching of signal sampling models; or
M. matching of interrogation radar signal with reference interrogation radar signal, and interrogation response signature with reference interrogation response signature using a neural network, such as a Convolutional Neural Network (CNN), configured for matching of signal sampling models.

48. The method according to claim 45, wherein the method comprises:
transmitting the interrogation radar signal towards a slave signal generator object, wherein the slave signal generator object comprises a Wideband Chaos Generating Material (WCGM), a Metamaterial interference object (MMIO), and/or a Natural Object (NO);
emitting, by the slave signal generator object, an altered interrogation radar signal, wherein the altered interrogation radar signal is emitted towards the target object; and
receiving, by the receiver antenna, the reflected radar signal from the target object in dependence on the altered interrogation radar signal.

49. The method according to claim 48, wherein the method comprises:
detecting and analyzing the slave signal generator object;
transmitting, by the transmitter antenna, an interrogation radar signal towards the slave signal generator object, and receiving, by the receiver antenna, a characteristic radar signal from the slave signal generator object; and
determining, by pattern matching and in dependence on the characteristic radar signal from the slave signal generator object, signal characteristics of the slave signal generator object.

50. The method according to claim 45, wherein radar signals follow along one or more of the following radar signal paths:
A. from the transmitter antenna to the target object to the receiver antenna;
B. from the transmitter antenna to the slave signal generator object to the receiver antenna;
C. from the transmitter antenna to the slave signal generator object to the target object to the receiver antenna; and
D. from the transmitter antenna to the slave signal generator object to the target object to the slave signal generator object to the receiver antenna.

51. The method according to claim 45, wherein the one or more properties is one or more physical or chemical parameters of the target object, optionally wherein the one or more properties comprise:
a volume of the target object;
a moisture level of the target object;
an amount of water or fluid in the target object;
a sugar or salt concentration of the target object; and/or
a chemical property or parameter of the target object, such as a concentration of one or more components of the target object and/or a substance composition of the target object.

52. The method according to claim 45, wherein the method comprises identifying a target object by matching the target object's frequency dependent interrogation response signature, with a reference interrogation response signature stored in the reference database;
optionally wherein the method comprises transmitting at least one adjusted interrogation radar signal based on a reference interrogation radar signal associated with the identified target object and stored in the reference database, wherein the adjusted interrogation radar signal result in a better differentiation of interrogation response signature for the target object than a previously transmitted interrogation radar signal.

53. The method according to any claim 45, wherein the method further comprises:
returning at least one look-up index to a matching, or new, data record in the reference database holding a reference pair of reference interrogation radar signal and reference interrogation response signature;
updating a target object, location, and categorization (TLC) data record; and
transferring the TLC data record to an external application, via an interface of the radar system for radar data consumption.

54. The method according to claim 53, wherein the TLC is configured to comprise a target object position, and at least one of:
A. target object's physical state such as closed, open, a temperature, strains, forces, and humidity;
B. target object's chemical properties, such as water sugar concentration;
C. target object's distance to antenna arrangements;
D. target object's angle direction as seen from the radar antenna arrangements,
E. target object's speed and movement direction in relation to the radar antenna arrangements;
F. a sampling model matching score rating the match between pairs of interrogation radar signal and interrogation response signature with reference pairs of reference interrogation radar signal and reference interrogation response signature stored in the reference database; and
G. the Target Object's reflected signal strength.

55. A millimeter radar system for remote substance analysis of a target object, the radar system comprising:
a waveform generator, configured to generate an interrogation radar signal;
a transmitter antenna configured to transmit the interrogation radar signal into a radar coverage space having at least one target objects;
a receiver antenna configured to receive a reflected radar signal from the target object;
a mixer, configured to receive the interrogation radar signal and the reflected radar signal for amplification and additive combination into a mixer signal;
a frequency domain signal transformer, configured to receive the mixer signal and to generate an interrogation response signature, wherein the interrogation response signature expresses energy distribution over a frequency plane; and
a pattern matching function configured to:
receive a time correlated interrogation radar signal and interrogation response signature,
match pairs of interrogation radar signal and interrogation response signature with pairs of reference interrogation radar signal and reference interrogation response signature stored in a reference database, and
determine one or more properties of the target object in dependence on data associated with the matched reference pair stored in the reference database.

56. The millimeter radar system according to claim 55, wherein the pattern matching function is further configured to:
return a look-up index, or indexes, of best matched previously recorded reference interrogation radar signal and reference interrogation response signature with characteristics in the reference database, if any, and
for each target object matched, compile a Target Localization and Categorization (TLC) data record.

57. The millimeter radar system according to claim 55, wherein the radar system further comprises an interface configured to offer reference database access to an application, process, simultaneously localization and mapping (SLAM), a radar console or supervision central, a vehicle anti-collision system, medical surveillance, caretaker service monitoring system, or any other system consuming at least one out of: target object localization position and categorization and target object's state data.

58. The millimeter radar system according to claim 55, wherein the radar system comprises a Single Input Single Output (SISO) transceiver antenna configuration; or
wherein the radar system comprises a Multiple Input Multiple Output (MIMO) transceiver antenna configuration; or
wherein the RADAR system is a Synthetic Aperture (SAR) RADAR, equipped with a Multiple Input Multiple Output (MIMO) antenna arrangement.

59. The millimeter radar system according to claim 55, wherein the radar system further comprises a slave signal generator object;
wherein the radar system is configured to transmit the interrogation radar signal towards the slave signal generator object, and the slave signal generator object is configured to, in dependence on the interrogation radar signal, transmit a second interrogation radar signal to the target object;
optionally wherein the slave signal generator object comprises a Wideband Chaos Generating Material (WCGM), a Metamaterial interference object (MMIO), or a Natural Object (NO);
optionally wherein the slave signal generator object is configured to receive a millimeter radar signal at a first signal strength and first frequency spectrum, and to emit a radar signal at a second signal strength, and a second frequency spectrum wherein the first frequency signal spectrum, and second frequency signal spectrum width differs.

60. The millimeter radar system according to claim 55, wherein the target object comprises a High Frequency Detection (HFD) diaper or wound dressing comprising a pocket with a fluid absorbing material, wherein the fluid absorbing material has a first radar frequency response intensity signature when dry, and a second radar frequency response intensity signature when a water-based fluid, such as a body fluid or urine, is absorbed;
wherein the millimeter radar system is configured to determine the absorbed fluid concentration of the HFD diaper or wound dressing, one or more substances absorbed by the HFD diaper or wound dressing, and/or one or more substance volumes absorbed by the HFD diaper or wound dressing.

61. The millimeter radar system according to claim 60, wherein the reference database stores reference interrogation radar signal and reference interrogation response signature pairs to describe HDF diaper or wound dressing signatures for different fluid concentrations, and/or identified type of fluid substances.

62. The millimeter radar system according to claim 60, wherein the system further comprises:
A. a diaper care surveillance system;
B. a warning GUI alarm system, arranged to alert care providers if a HFD Diaper or wound dressing needs to be shifted;
C. an optional ID marker system, that lets care provider register new HFD Diapers or wound dressings; and
D. a Follow up system, configured to receive messages from the care provider, and care receiver.

63. The millimeter radar system according to claim 55, wherein the target object comprises:
a substance having a non-linear frequency dependent radio signature, such as a carbon hydrate, or a sugar and water solution; and
an enclosure protecting and preventing the substance from evaporation or drying while being at least partially radar transparent.

64. The millimeter radar system according to any claim 11, wherein the target object comprises:
a carbon hydrate solution, such as a sugar and water solution, with a radar frequency response signature dependent on a concentration of the carbon hydrate solution, optionally dependent on a Brix grade (Bx°) value of the carbon hydrate solution.
